(19) United States
(12) Patent Application Publication
(10) Pub. No.: US 2025/0333371 A1
(43) Pub. Date: Oct. 30, 2025

(54) COMPOUNDS

(71) Applicant: PSYLO PTY LTD, Sydney (AU)

(72) Inventors: Samuel BANISTER, Sydney (AU); Willam JORGENSEN, Sydney (AU); Jinlong TAN, Sydney (AU); Lachlan WHISH, Sydney (AU)

(21) Appl. No.: 18/868,244

(22) PCT Filed: May 30, 2023
(86) PCT No.: PCT/AU2023/050455

§ 371 Date: Nov. 22, 2024

(30) Foreign Application Priority Data
May 30, 2022 (AU) 2022901458

(51) Int. Cl. C07C 211/30; A61K 31/137; A61K 31/47; C07C 211/35; C07C 215/52; C07C 217/60; C07D 215/227

(52) U.S. Cl. CPC C07C 211/30; A61K 31/137; A61K 31/47; C07C 211/35; C07C 215/52; C07C 217/60; C07D 215/227

(57) ABSTRACT

The present disclosure relates to compounds of formula (I), their methods of synthesis, and their use in the treatment of mental illness or central nervous system disorders.

This application claims priority to Australian provisional application no. 2022901458 (filed on 30 May 2022), the entire contents of which are incorporated herein by reference.

### FIELD OF THE INVENTION

The present disclosure relates generally to compounds active at the serotonergic 5-HT_{2A} receptor that may be useful in the treatment of mental illness or central nervous system disorders. The disclosure also relates to methods of synthesis of the compounds, compositions comprising the compounds and to methods for their use.

### BACKGROUND OF THE INVENTION

Mental illness covers many neuropsychiatric disorders which cause enormous burden on the lives of their sufferers. Diagnoses such as treatment resistant depression, major depressive disorder, eating disorders, substance abuse disorders, post-traumatic stress disorder, obsessive compulsive disorder, attention deficit disorders, schizophrenia, and others can cause such devastating symptoms that many sufferers lose the capability of leading a normal life.

A variety of serotonergic drugs such as antidepressants, serotonin reuptake inhibitors, monoamine oxidase inhibitors, selective serotonin reuptake inhibitors, and others are commercially available to treat mental illnesses. Unfortunately, in many indications, these therapeutics provide limited benefit when compared to a placebo. Additionally, these therapeutics can result in a wide range of side effects including loss of libido, insomnia, fatigue, weight gain, and others. In spite of their limited efficacy, these drugs continue to be used to treat neuropsychiatric conditions as well as a broad range of auxiliary medical indications. There have been limited advances in new treatment options since many of these drugs were released, and the pharmaceutical industry has come under increased financial pressure to de-emphasise neuroscience programmes entirely. The unmet need for more efficacious mental health treatment is on the rise, and the global COVID-19 pandemic is likely to increase disease burden around the world.

In the 1950s and 1960s, the use of psychedelic drugs to treat various mental illnesses was extensively explored, and these substances showed promise as treatments for many diseases of the central nervous system (CNS). Following decades of prohibition, scientific research into the application of psychedelics as treatments for mental illnesses has been gaining momentum. The serotonergic psychedelic agent psilocybin has been designated a Breakthrough Therapy by the FDA for the treatment of major depressive disorder (2019) and treatment-resistant depression (2018). Psilocybin is the prodrug compound produced by more than many species of mushrooms known collectively as psilocybin mushrooms or “magic mushrooms”. Psilocybin is rapidly metabolized to the bioactive compound psilocin, which produces a state of altered consciousness including changes in perception, visual hallucinations, and distorted sense of space, time, and self. Many patients report spiritual or “mystical” experiences which have profound and lasting impact on the patients' mood and behaviour. Psilocybin has shown promise in more than 50 clinical trials for neuropsychiatric indications, including numerous anxiety disorders, obsessive-compulsive disorder, anorexia nervosa, alcohol dependence, and tobacco addiction. Psilocybin and other psychedelic compounds such as N,N-dimethyltryptamine (DMT) and 5-methoxy-N,N-dimethyltryptamine (5-MeO-DMT) have both immediate and persistent effects on mental state, with the latter extending far beyond the duration of action, possibly as a result of their ability to incite increased neuroplasticity, promote neural outgrowth, and increase spine density of the synaptic neurons in the brain.

To date, psilocybin remains classified as a controlled substance and/or drug of abuse in most countries under national drug laws. However, clinical investigations have recently led to increased awareness of the potential for psychedelic drugs as breakthrough therapies to treat CNS diseases of enormous unmet medical need.

Despite its therapeutic potential, psilocybin and other psychedelics remain scheduled drugs of abuse in most countries and the commercial path to market for these drugs as medicines is uncertain. As an adjunct to psychotherapy, the long duration of action of psilocybin and LSD make treatment sessions costly and impractical for broad implementation. In spite of a long history of safe human use, several adverse events have been reported in clinical trials, and it is possible that these may be attributed to signalling bias at 5-HT_{2A} (the primary target) or off-target activity at, for example, 5-HT_{2B} receptors (a cardiac liability antitarget) or 5-HT_{1A} (an anxiolytic target) or 5-HT_{2C} receptors (a disease-relevant target for obesity and some genetic epilepsies, for example). Naturally-occurring psychedelics provide important lead structures for a new generation of neurotherapeutic agents with novel mechanisms of action and/or superior clinical efficacy to currently available neuropsychiatric medications.

In view of the foregoing there is an ongoing need to develop new compounds which may be useful in the treatment of mental illness or central nervous system disorders.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect the present disclosure provides a compound of formula (I):

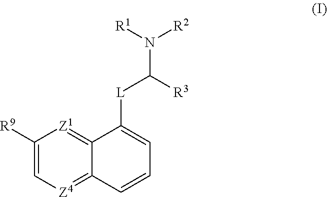

wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl, said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ alkoxy, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² together with the nitrogen atom to which they are attached form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² together with the atoms to which they are attached form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
Z¹ is CR⁸ or N;
Z⁴ is CR¹¹ or N;
R⁸, R⁹ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

In any aspect or embodiment described herein, a compound of the invention may be provided in the form of a pharmaceutically acceptable salt, solvate, tautomer, N-oxide, stereoisomer, metabolite, polymorph and/or prodrug thereof.

In another aspect the present disclosure provides a medicament comprising a compound of formula (I) according to any one of the herein disclosed embodiments, or a pharmaceutically acceptable salt, solvate, tautomer, N-oxide, stereoisomer, metabolite, polymorph and/or prodrug thereof

In another aspect the present disclosure provides a pharmaceutical composition comprising a compound of formula (I) according to any one of the herein disclosed embodiments, or a pharmaceutically acceptable salt, solvate, tautomer, N-oxide, stereoisomer, metabolite, polymorph and/or prodrug thereof, and a pharmaceutically acceptable excipient.

In another aspect the present disclosure provides a method of treating a disease, disorder or condition associated with activity of a serotonin receptor, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph and/or prodrug thereof.

In another aspect the present disclosure provides a method of treating a disease, disorder and/or condition associated with activity of a serotonin receptor, the method comprising administering to a subject in need thereof a therapeutically effective amount of a compound of formula (I) as described herein, or a pharmaceutically acceptable salt, solvate, tautomer, N-oxide, stereoisomer, metabolite, polymorph and/or prodrug thereof, in combination with a therapeutically effective amount of another agent useful for treatment of said disease, disorder or condition.

In another aspect the present disclosure provides a method of treating a mental illness, the method comprising administering to a subject in need thereof a therapeutically effective amount of a compound of formula (I) as described herein, or a pharmaceutically acceptable salt, solvate, tautomer, N-oxide, stereoisomer, metabolite, polymorph and/or prodrug thereof.

In some embodiments, the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

In another aspect the present disclosure provides a method for treating a central nervous system (CNS) disease, disorder or condition and/or a neurological disease, disorder or condition, the method comprising administering to a subject in need thereof a therapeutically effective amount of a compound of formula (I) as described herein, or a pharmaceutically acceptable salt, solvate, tautomer, N-oxide, stereoisomer, metabolite, polymorph and/or prodrug thereof.

In some embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect the present disclosure provides a method for increasing neuronal plasticity and/or increasing dendritic spine density, the method comprising contacting a neuronal cell with an effective amount of a compound of formula (I) as described herein, or a pharmaceutically acceptable salt, solvate, tautomer, N-oxide, stereoisomer, metabolite, polymorph and/or prodrug thereof, to thereby increase neuronal plasticity and/or increase dendritic spine density of the neuronal cell.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention; weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect the present disclosure provides a method for activating a serotonin receptor in a cell, either in a biological sample or in a patient, comprising administering a compound of formula (I) as described herein, or a pharmaceutically acceptable salt, solvate, tautomer, N-oxide, stereoisomer, metabolite, polymorph and/or prodrug thereof, to the cell.

The present disclosure is not to be limited in scope by the specific embodiments described herein, which are intended for the purpose of exemplification only. Functionally-equivalent products, compositions and methods are clearly within the scope of the invention, as described herein.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

### Definitions

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

The terms “treatment” or “treating” of a subject includes delaying, slowing, stabilizing, curing, healing, alleviating, relieving, altering, remedying, less worsening, ameliorating, improving, or affecting the disease or condition, the sign or symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of signs or symptoms or making the injury, pathology or condition more tolerable to the individual; slowing in the rate of degeneration or decline; making the final point of degeneration less debilitating.

In particularly preferred embodiments, the methods of the present invention can be to prevent or reduce the severity, or inhibit or minimise progression, of a sign or symptom of a disease or condition as described herein. As such, the methods of the present invention have utility as treatments as well as prophylaxes.

As used herein, “preventing” or “prevention” is intended to refer to at least the reduction of likelihood of the risk of (or susceptibility to) acquiring a disease or disorder (i.e., causing at least one of the clinical signs or symptoms of the disease not to develop in an individual that may be exposed to or predisposed to the disease but does not yet experience or display signs or symptoms of the disease). Biological and physiological parameters for identifying such patients are provided herein and are also well known by physicians.

As used herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

The term “selective” means a greater activity against a first target (e.g., a first 5-HT receptor subtype) relative to a second target (e.g., a second 5-HT receptor subtype). In some embodiments a compound has a selectivity of at least 1.25-fold, at least 1.5 fold, at least 2-fold, at least 3-fold, at least 4-fold, at least 5-fold, at least 6-fold, at least 10-fold or at least 100-fold greater towards a first target relative to a second target. In some embodiments, a compound described herein is selective towards the 5-HT_{2A} receptor relative to one or more other 5-HT receptor subtypes such as 5-HT_{2B} and/or 5-HT_{2C}, preferably 5-HT_{2B}. In some embodiments, a compound described herein is selective towards the 5-HT_{2C} receptor relative to one or more other 5-HT receptor subtypes such as 5-HT_{2A} and/or 5-HT_{2B}, preferably 5-HT_{2B}.

Various features of the present disclosure are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, ±5%, ±1% or ±0.1% of that value.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents. Examples of “alkyl” as used herein include, but are not limited to, methyl, ethyl, n-propyl, isopropyl, n-butyl, isobutyl, t-butyl, n-pentyl, isopentyl, and the like

As used herein, the terms “C₁-C₂ alkyl”, “C₁-C₃ alkyl” and “C₁-C₆ alkyl” refer to an alkyl group, as defined herein, containing at least 1, and at most 2, 3 or 6 carbon atoms respectively, or any range in between (eg alkyl groups containing 2-5 carbon atoms are also within the range of C₁-C₆).

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene The term “alkenyl” whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆ alkylene means an alkylene group having 2, 3, 4, 5 or 6 carbon atoms. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆ alkynyl means an alkynyl group having 2, 3, 4, 5 or 6 carbon atoms. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

The term “cycloalkyl” is intended to include mono-, bi- or tricyclic alkyl groups. The number of carbon atoms that are possible in the referenced cycloalkyl group are indicated by the prefix “Cₙ₁₋ₙ₂”. For example, the term C₃₋₈ cycloalkyl means a cycloalkyl group having 3, 4, 5, 6, 7 or 8 carbon atoms. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “alkylenecycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined herein. The numerical range from x to y in “C_{x-y} alkylenecycloalkyl” relates to the total number of alkyl carbons and cycloalkyl ring atoms. Exemplary alkylenecycloalkyl groups include, but are not limited to, methylenecyclopropyl, methylenecyclobutyl, methylenecyclopentyl and methylenecyclohexyl.

The term “aryl” refers to an aromatic ring system having any suitable number of ring atoms and any suitable number of rings. The number of carbon atoms that are possible in the referenced aryl group are indicated by the prefix “Cₙ₁₋ₙ₂”. For example, the term C₆₋₁₂ aryl means an aryl group having 6, 7, 8, 9, 10, 11 or 12 carbon atoms. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

The term “alkylenearyl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above.

The numerical range from x to y in “C_{x-y} alkylenearyl” relates to the total number of alkyl carbons and aryl ring atoms. Examples of alkylenearyl groups include, but are not limited to, benzyl and ethylenephenyl.

As used herein, the term “alkoxy” refers to an alkyl group as defined herein covalently bound via an O linkage. The alkoxy group is optionally substituted with substituents. Examples of “alkoxy” as used herein include, but are not limited to methoxy, ethoxy, propoxy, isoproxy, butoxy, iso-butoxy, tert-butoxy and pentoxy.

As used herein, the terms “C₁-C₂ alkoxy”, “C₁-C₃ alkoxy” and “C₁-C₆ alkoxy” refer to an alkoxy group, as defined herein, containing at least 1, and at most 2, 3 or 6 carbon atoms respectively, or any range in between (eg alkoxy groups containing 2-5 carbon atoms are also within the range of C₁-C₆).

As used herein, the term “alkylamine” refers to an alkyl group as defined herein having one or more amino groups. The amino groups can be primary, secondary or tertiary. The alkyl amine can be further substituted with a hydroxy group to form an amino-hydroxy group. Examples of alkylamines include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group.

As used herein, the terms “C₁-C₂ alkylamine”, “C₁-C₃ alkylamine” and “C₁-C₆ alkylamine” refer to an alkylamine group, as defined herein, containing at least 1, and at most 2, 3 or 6 carbon atoms respectively, or any range in between (e.g., alkylamine groups containing 2-5 carbon atoms are also within the range of C₁-C₆).

As used herein, the term “alkylsulfonyl” refers to an alkyl group as defined herein having one or more sulfonyl groups. The sulfonyl group can link the alkylsulfonyl to the point of attachment with the rest of the compound, be at the omega position of the alkyl group, or link together at least two carbon atoms of the alkyl group.

As used herein, the terms “C₁-C₂ alkylsulfonyl”, “C₁-C₃ alkylsulfonyl” and “C₁-C₆ alkylsulfonyl” refer to an alkylsulfonyl group, as defined herein, containing at least 1, and at most 2, 3 or 6 carbon atoms respectively, or any range in between (e.g., alkylsulfonyl groups containing 2-5 carbon atoms are also within the range of C₁-C₆).

The term “heteroatom” as used herein means an atom of any element other than carbon or hydrogen. Examples of heteroatoms include nitrogen, oxygen, sulfur and phosphorus. Preferred heteroatoms include N, O and S, preferably N and O.

The term “heteromoiety” as used herein means a chemical group comprising a heteroatom. Examples of heteromoieties include O, S, S(O), SO₂, N and NH.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, ie, a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable substituents for a particular functional group will be apparent to those skilled in the art.

Examples of substituents include but are not limited to C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halo, ureido, C₁-C₆ perfluoroalkyl. Preferably the substituents include amino, halo, C₁-C₆ alkyl, amido, hydroxyl.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is fluoro or chloro.

As used herein, the term “haloalkyl” refers to an alkyl group as defined herein in which one or more (up to all) of the available hydrogen atoms have been replaced with a halogen. In some instances, the term“perfluoro” can be used to define a compound or radical where all the hydrogens are replaced with fluorine. For example, perfluoromethyl refers to 1,1,1-trifluoromethyl.

As used herein, the terms “C₁-C₂ haloalkyl”, “C₁-C₃ haloalkyl” and “C₁-C₆ haloalkyl” refer to an haloalkyl group, as defined herein, containing at least 1, and at most 2, 3 or 6 carbon atoms respectively, or any range in between (e.g. haloalkyl groups containing 2-5 carbon atoms are also within the range of C₁-C₆).

For example a C₁ haloalkyl group could be, but is not limited to, fluoromethyl, or difluoromethyl, or trifluoromethyl.

As used herein, the term “haloalkenyl” refers to an alkenyl group as defined above in which one or more of the available hydrogen atoms have been replaced with a halogen. Thus, for example, “C₁₋₆ haloalkenyl” (or “C₁-C₆ haloalkenyl”) refers to a C₁ to C₆ linear or branched alkenyl group as defined above with one or more halogen substituents.

As used herein, the term “haloalkynyl” refers to an alkynyl group as defined above in which one or more of the available hydrogen atoms have been replaced with a halogen. Thus, for example, “C₁₋₆ haloalkynyl” (or “C₁-C₆ haloalkynyl”) refers to a C₁ to C₆ linear or branched alkynyl group as defined above with one or more halogen substituents.

As used herein the term haloalkoxy refers to an alkoxy group as defined herein substituted with at least one halogen.

The term “amino” or “amine” refers to the group —NH₂.

The term “substituted amino” or “secondary amino” refers to an amino group having a hydrogen replaced with, for example a C₁-C₆ alkyl group (“C₁-C₆ alkylamino”), an aryl or aralkyl group (“arylamino”, “aralkylamino”) and so on. C₁-C₃ alkylamino groups are preferred, such as for example, methylamino (NHMe), ethylamino (NHEt) and propylamino (NHPr).

The term “disubstituted amino” or “tertiary amino” refers to an amino group having the two hydrogens replaced with, for example a C₁-C₆alkyl group, which may be the same or different (“dialkylamino”), an aryl and alkyl group (“aryl(alkyl)amino”) and so on. Di(C₁-C₃alkyl)amino groups are preferred, such as for example, dimethylamino (NMe₂), diethylamino (NEt₂), dipropylamino (NPr₂) and variations thereof (eg N(Me)(Et) and so on).

The term “nitro” refers to the group —NO₂.

The term “cyano” and “nitrile” refer to the group —CN.

The term “amido” or “amide” refers to the group —C(O)NH₂.

The term “substituted amido” or “substituted amide” refers to an amido group having a hydrogen replaced with, for example a C₁-C₆ alkyl group (“C₁-C₆ alkylamido” or “C₁-C₆ alkylamide”), an aryl (“arylamido”), aralkyl group (“aralkylamido”) and so on. C₁-C₃ alkylamide groups are preferred, such as for example, methylamide (—C(O)NHMe), ethylamide (—C(O)NHEt) and propylamide (—C(O)NHPr) and includes reverse amides thereof (eg NHMeC(O)—, —NHEtC(O)— and —NHPrC(O)—).

The term “disubstituted amido” or “disubstituted amide” refers to an amido group having the two hydrogens replaced with, for example a C₁-C₆alkyl group (“di(C₁-C₆ alkyl)amido” or “di(C₁-C₆ alkyl)amide”), an aralkyl and alkyl group (“alkyl(aralkyl)amido”) and so on. Di(C₁-C₃ alkyl)amide groups are preferred, such as for example, dimethylamide (—C(O)NMe₂), diethylamide (—C(O)NEt₂) and dipropylamide ((—C(O)NPr₂) and variations thereof (eg C(O)N(Me)Et and so on) and includes reverse amides thereof.

The term “sulfonyl” refers to the group —SO₂H.

The term “substituted sulfonyl” refers to a sulfonyl group having the hydrogen replaced with, for example a C₁-C₆ alkyl group (“sulfonylC₁-C₆ alkyl”), an aryl (“arylsulfonyl”), an aralkyl (“aralkylsulfonyl”) and so on. Sulfonyl C₁-C₃ alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁-C₆ alkyl group (“sulfonylamidoC₁-C₆ alkyl”), an aryl (“arylsulfonamide”), aralkyl (“aralkylsulfonamide”) and so on. SulfonylamidoC₁-C₃ alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁-C₆ alkyl group, which may be the same or different (“sulfonylamidodi(C₁-C₆ alkyl)”), an aralkyl and alkyl group (“sulfonamido(aralkyl)alkyl”) and so on. Sulfonylamidodi(C₁-C₃ alkyl) groups are preferred, such as for example, —SO₂NMe₂, —SO₂NEt₂ and —SO₂NPr₂ and variations thereof (eg —SO₂N(Me)Et and so on) and includes reserve sulfonamides thereof (eg —N(Me)SO₂Me and so on).

The term “sulfate” refers to the group OS(O)₂OH and includes groups having the hydrogen replaced with, for example a C₁-C₆ alkyl group (“alkylsulfates”), an aryl (“arylsulfate”), an aralkyl (“aralkylsulfate”) and so on. C₁-C₃ alkylsulfates are preferred, such as for example, OS(O)₂OMe, OS(O)₂OEt and OS(O)₂OPr.

The term “sulfonate” refers to the group SO₃H and includes groups having the hydrogen replaced with, for example a C₁-C₆ alkyl group (“alkylsulfonate”), an aryl (“arylsulfonate”), an aralkyl (“aralkylsulfonate”) and so on. C₁-C₃ alkylsulfonates are preferred, such as for example, SO₃Me, SO₃Et and SO₃Pr.

The term “amino acid” as herein defined refers to a moiety containing an amino group and a carboxyl group linked by at least one carbon. An amino acid may refer a natural or non-natural amino acid, preferably a natural amino acid such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, preferably the amino acid is arginine, lysine or histidine, most preferably lysine.

The term “carboxylate” or “carboxyl” refers to the group —COO— or —COOH.

The term “carbamate” or “carbomyl” refers to the group —OC(O)NH₂. The carbamate may be substituted, or may be disubstituted, for example with an alkyl group such as but not limited to C₁-C₆ alkyl.

The term “carbonate” refers to the group —OC(O)O— or —OC(O)OH.

The term “alkylcarbonate” as herein defined refers to a carbonate group having the hydrogen replaced with, for example a C₁-C₆ alkyl group, an aryl or aralkyl group (“arylcarbonate” or “aralkylcarbonate”) and so on. CO₃C₁-C₃alkyl groups are preferred, such as for example, methylcarbonate (CO₃Me), ethylcarbonate (CO₃Et) and propylcarbonate (CO₃Pr).

The term “ester” refers to a carboxyl group having the hydrogen replaced with, for example a C₁-C₆ alkyl group (“carboxylC₁-C₆ alkyl” or “alkylester”), an aryl or aralkyl group (“arylester” or “aralkylester”) and so on. CO₂C₁-C₃ alkyl groups are preferred, such as for example, methylester (CO₂Me), ethylester (CO₂Et) and propylester (CO₂Pr) and includes reverse esters thereof (eg —OC(O)Me, —OC(O)Et and —OC(O)Pr).

The term “heterocyclyl” refers to a moiety obtained by removing a hydrogen atom from a ring atom of a heterocyclic compound which moiety has from 3 to 12 ring atoms (unless otherwise specified), of which 1, 2, 3, 4 or more are ring heteroatoms, for example independently selected from O, S and N, or ring heteromoieties, for example independently selected from O, S, S(O), SO₂, N and NH. When a heterocyclyl group contains the prefix Cₙ₁₋ₙ₂ or “n1 to n2” this prefix indicates the number of carbon atoms in the corresponding carbocyclic group, in which one or more, suitably 1, 2, 3, 4 or more, of the ring atoms is replaced with a heteroatom or heteromoiety.

In this context, the prefixes 3-, 4-, 5-, 6-, 7-, 8-, 9- and 10-membered denote the number of ring atoms, or range of ring atoms, whether carbon atoms or heteroatoms. For example, the term “C₃₋₁₀ heterocyclyl” or “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (eg 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls also encompass heteroaryl (aromatic heterocyclyls) and heterocycloalkyl (non-aromatic heterocyclyls). Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. When a heteroaryl group contains the prefix Cₙ₁₋ₙ₂ or “n1 to n2” this prefix indicates the number of carbon atoms in the corresponding aryl group, in which one or more, suitably 1, 2, 3, 4 or more, of the ring atoms is replaced with a heteroatom. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (eg bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, napthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoiine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “heterocycloalkyl” or “non-aromatic heterocyclyl” encompasses optionally substituted saturated and unsaturated rings which contain at least one heteroatom such as N, S and O, or a heteromoiety such as O, S, S(O), SO₂, N and NH. The ring may contain 1, 2, 3, 4 or more heteroatoms or heteromoieties. When a heterocycloalkyl group contains the prefix Cₙ₁₋ₙ₂ or “n1 to n2” this prefix indicates the number of ring atoms, in which one or more, suitably 1, 2, 3, 4 or more, of the ring atoms is replaced with a heteroatom or heteromoiety. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-8 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “alkyleneheteroaryl” refers to a radical having an alkyl component and a heteroaryl component, where the alkyl component links the heteroaryl component to the point of attachment. The alkyl component is as defined above, except that the alkyl component is at least divalent (eg an alkylene) to link to the heteroaryl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined herein. The numerical range from x to y in “C_{x-y} alkylenecycloalkyl” relates to the total number of alkyl carbons and heteroaryl ring atoms (carbon and heteroatoms together).

The term “alkyleneheterocycloalkyl” refers to a radical having an alkyl component and a heterocycloalkyl component, where the alkyl component links the heterocycloalkyl component to the point of attachment. The alkyl component is as defined above, except that the alkyl component is at least divalent (eg an alkylene) to link to the heterocycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component may be any heterocycloalkyl group as defined herein. The numerical range from x to y in “C_{x-y}alkyleneheterocycloalkyl” relates to the total number of alkyl carbons and heterocycloalkyl ring atoms (carbon and heteroatoms together).

The term “pharmaceutically acceptable” may be used to describe any salt, solvate, tautomer, N-oxide, stereoisomer and/or prodrug thereof, or any other compound which upon administration to a subject, is capable of providing (directly or indirectly) a compound as described herein or an active metabolite or residue thereof and typically that is not deleterious to the subject.

As used herein, the term “solvate” refers to a complex of the compound and either stoichiometric or non-stoichiometric amounts of a solvent. Solvates are often formed during the process of crystallization with pharmaceutically acceptable solvents such as water, ethanol, and the like. Hydrates are formed when the solvent is water, or alcoholates are formed when the solvent is alcohol.

As used herein, the term “polymorph” refers to the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

As used herein, the term “metabolite” refers to a derivative of a compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. Metabolites of the compounds disclosed herein are optionally identified either by administration of compounds to a host and analysis of tissue samples from the host, or by incubation of compounds with hepatic cells in vitro and analysis of the resulting compounds.

As used herein, a “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of formula (I). The amino acid residues include the 20 naturally occurring amino acids commonly designated by three letter symbols and also include, 4-hydroxyproline, hydroxylysine, demosine, isodemosine, 3-methylhistidine, norylin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula (I) through the carbonyl carbon prodrug sidechain.

The compound of formula (I) may demonstrate tautomerism. Tautomers are two interchangeable forms of a molecule that typically exist within an equilibrium. Any tautomers of the compound of formula (I) are to be understood as being within the scope of the invention.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. The term “stereoisomers” refers to compounds which have identical chemical constitution, but differ with regard to the arrangement of the atoms or groups in space. As used herein, the term “stereoisomer” includes but is not limited to diastereomers, enantiomers and atropisomers, as well as mixtures thereof such as racemic mixtures. All stereoisomers of the compound of formula (I) are within the scope of the invention. In some embodiments, the compound is a stereoisomerically enriched form of the compound of formula (I) at any stereocentre. The compound may be enriched in one stereoisomer over another by at least about 60, 70, 80, 90, 95, 98 or 99%.

The salts of compound of formula (I) are preferably pharmaceutically acceptable, but it will be appreciated that non-pharmaceutically acceptable salts also fall within the scope of the present disclosure, for example, as these may be useful as intermediates in the preparation of pharmaceutically acceptable salts or in methods not requiring administration to a subject.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Plasma concentrations of 1-7 in male C57BL/6 mice following intraperitoneal (IP) administration at 10 mg/kg, as described in Example 49.

FIG. 2: Plasma concentrations of 1-15 in male C57BL/6 mice following IP administration at 10 mg/kg, as described in Example 49.

FIG. 3: Time binned and mean±SD (n=3) Head Twitch Response (HTR) counts of 1-7 in C57BL/6 mice following subcutaneous (SC) administration over several doses, as described in Example 49.

FIG. 4: Temperature and locomotor results displayed as mean±standard deviation (SD) (n=3) HTR counts of 1-7 in C57BL/6 mice following SC administration over several doses, as described in Example 49.

### COMPOUNDS

The disclosure relates to compounds of formula (I) that have surprisingly shown activity at least at one serotonin receptor subtype. Preferred embodiments are selective for either the 5-HT_{2A} or the 5-HT_{2C} receptor, typically selective for 5-HT_{2A} or 5-HT_{2C} over 5-HT_{2B} subtypes and may be further selective for 5-HT_{2A} over 5-HT_{2C} or for 5-HT_{2C} over 5-HT_{2A}. The activity of these compounds is particularly surprising given the unique structure of the 6,6-bicyclic compounds as defined in formula (I).

The present disclosure provides compounds of formula (I):

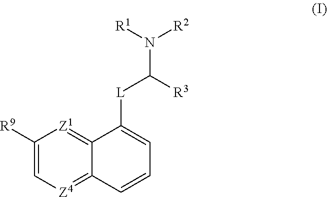

or a pharmaceutically acceptable salt, solvate, tautomer, N-oxide, stereoisomer, metabolite, polymorph and/or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇-1l alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ alkoxy, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² together with the nitrogen atom to which they are attached form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² together with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
Z¹ is CR⁸ or N;
Z⁴ is CR¹¹ or N;
R⁸, R⁹ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.
   R¹ and R²

In some embodiments, the C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl at R¹ and/or R² is further optionally substituted with a substituent independently selected from (O), C₁₋₆ alkyl, C₁₋₆ alkoxy, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴.

In some embodiments, R¹ and R² are each independently selected from selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₇₋₁₈ alkylenearyl and C₄₋₁₄ alkylenecycloalkyl.

In some embodiments, R¹ and R² are each independently selected from selected from hydrogen, C₁₋₆ alkyl, C₃₋₈ cycloalkyl, and C₇-1l alkylenearyl.

In some embodiments, R¹ and R² are each independently selected hydrogen, C₁₋₄ alkyl, C₃₋₄ cycloalkyl and O₇-8 alkylenearyl.

In some embodiments, R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl.

In some embodiments, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl.

In some embodiments, R¹ and R² are each independently selected from hydrogen, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl.

In some embodiments, R¹ and R² are each independently selected from C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl.

In some embodiments, both of R¹ and R² are hydrogen. In some embodiments, one of R¹ and R² is hydrogen. In some embodiments, neither of R¹ and R² are hydrogen.

In some embodiments, the C₁₋₆ alkyl, C₁₋₆ haloalkyl, C₂₋₆ alkenyl, C₂₋₆ haloalkenyl, C₂₋₆ alkynyl, C₂₋₆ haloalkynyl at R¹ and/or R² is linear. In some embodiments, the C₁₋₆ alkyl, C₁₋₆ haloalkyl, C₂₋₆ alkenyl, C₂₋₆ haloalkenyl, C₂₋₆ alkynyl, C₂₋₆ haloalkynyl at R¹ and/or R² is branched.

In some embodiments, wherein one or both of R¹ and R² is C₁₋₆ alkyl, one or both of the C₁₋₆ alkyl is linear. In some embodiments, wherein one or both of R¹ and R² is C₁₋₆ alkyl, one of the C₁₋₆ alkyl is linear. In some embodiments, wherein one or both of R¹ and R² is C₁₋₆ alkyl, both of the C₁₋₆ alkyl are linear. In some embodiments, wherein one or both of R¹ and R² is C₁₋₆ alkyl, one of the C₁₋₆ alkyl is branched. In some embodiments, wherein one or both of R¹ and R² is C₁₋₆ alkyl, one or both of the C₁₋₆ alkyl are branched. In some embodiments, wherein both of R¹ and R² are C₁₋₆ alkyl, one of the C₁₋₆ alkyl is linear and the other C₁₋₆ alkyl is branched.

In some embodiments, wherein one or both of R¹ and R² is C₁₋₄ alkyl (preferably C₁₋₃ alkyl), one or both of the C₁₋₄ alkyl (preferably C₁₋₃ alkyl) is linear. In some embodiments, wherein one or both of R¹ and R² is C₁₋₄ alkyl (preferably C₁₋₃ alkyl), one of the C₁₋₄ alkyl (preferably C₁₋₃ alkyl) is linear. In some embodiments, wherein one or both of R¹ and R² is C₁₋₄ alkyl (preferably C₁₋₃ alkyl), both of the C₁₋₄ alkyl (preferably C₁₋₃ alkyl) are linear.

In some embodiments, wherein one or both of R¹ and R² is C₁₋₄ alkyl (preferably C₁₋₃ alkyl), one of the C₁₋₄ alkyl (preferably C₁₋₃ alkyl) is branched. In some embodiments, wherein one or both of R¹ and R² is C₁₋₄ alkyl (preferably C₁₋₃ alkyl), one or both of the C₁₋₄ alkyl (preferably C₁₋₃ alkyl) are branched. In some embodiments, wherein both of R¹ and R² are C₁₋₄ alkyl (preferably C₁₋₃ alkyl), one of the C₁₋₄ alkyl (preferably C₁₋₃ alkyl, more preferably methyl) is linear and the other C₁₋₄ alkyl (preferably C₁₋₃ alkyl) is branched.

In some embodiments, the C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl at R¹ and/or R² is unsubstituted. In some embodiments, the C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl at R¹ and/or R² is substituted, preferably meta-substituted relative to the alkylene group (if the group specifies an alkylene group) or the nitrogen atom connected to R¹/R² (if the group does not specify an alkylene group).

In some embodiments, one of R¹ and R² is C₇₋₁₈ alkylenearyl, preferably C₇₋₈alkylenearyl, more preferably benzyl. In some embodiments, one of R¹ and R² is C₇₋₁₈ alkylenearyl, preferably C₇₋₈alkylenearyl, more preferably benzyl; and the other of R¹ and R² is hydrogen.

In some embodiments, wherein one or both of R¹ and R² is C₇₋₁₈ alkylenearyl, the C₇₋₁₈ alkylenearyl is unsubstituted. In some embodiments, wherein one or both of R¹ and R² is C₇₋₁₈ alkylenearyl, the C₇₋₁₈ alkylenearyl is substituted with one or more halogen, C₁₋₆ alkyl, C₁₋₆ alkoxy, C₁₋₆ haloalkyl; preferably one or more halogen, C₁₋₆ alkyl, C₁₋₆ alkoxy; more preferably fluoro, methyl, methoxy.

In some embodiments, one of R¹ and R² is C₃₋₈ cycloalkyl, preferably C₃₋₄ cycloalkyl, more preferably C₃ cycloalkyl. In some embodiments, one of R¹ and R² is C₃₋₈ cycloalkyl, preferably C₃₋₄ cycloalkyl, more preferably C₃ cycloalkyl; and the other of R¹ and R² is C₁₋₆ alkyl, preferably C₁₋₃ alkyl, more preferably C₁ alkyl.

In some embodiments, wherein one or both of R¹ and R² is C₃₋₈ cycloalkyl or C₄₋₁₄ alkylenecycloalkyl, preferably C₃₋₈ cycloalkyl; the C₃₋₈ cycloalkyl or C₄₋₁₄ alkylenecycloalkyl, preferably C₃₋₈ cycloalkyl, is unsubstituted.

In some embodiments, R¹ and/or R² are unsubstituted. In some embodiments, R¹ and R² are unsubstituted. In some embodiments, R¹ and/or R² are substituted. In some embodiments, R¹ and R² are substituted.

In some embodiments, R¹ and R² are the same. In other embodiments, R¹ and R² are different such that the nitrogen atom to which R¹ and R² is attached is an assymetric non-cyclic amine.

In some embodiments, R¹ and R², together with the nitrogen atom to which they are attached, form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴. In such embodiments, R¹ and R² together with the nitrogen atom to which they are attached for a cyclic amine.

In some embodiments, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

In some embodiments, R¹ and R² are each independently selected from C₁₋₄ alkyl. In some embodiments, R¹ and R² are each independently selected from C₁₋₃ alkyl. In some embodiments, R¹ and R² are each independently selected from C₁₋₂ alkyl. In some embodiments, R¹ and R² are both methyl.

In some embodiments, R¹ and R², together with the nitrogen to which they are attached, form any one of the following:

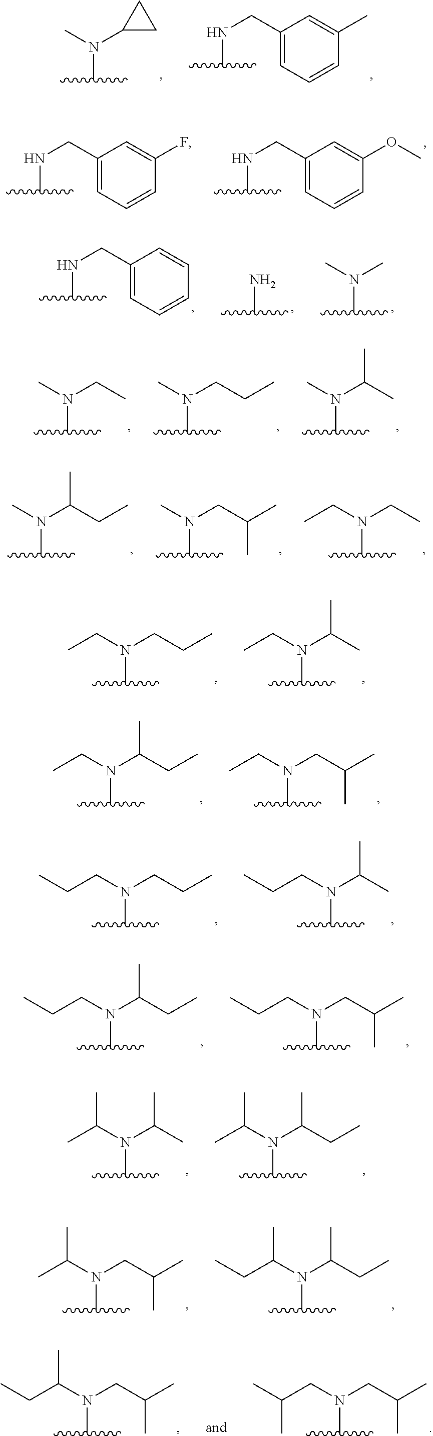

In some embodiments, R¹ and R², together with the nitrogen to which they are attached, form any one of the following:

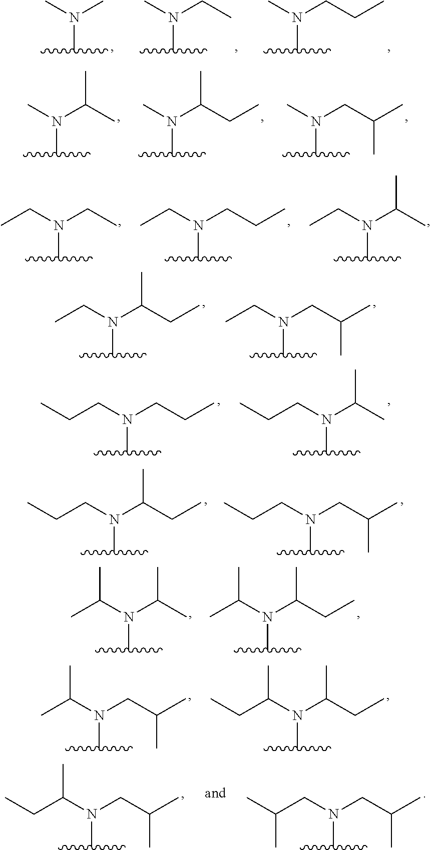

In some embodiments, R¹ and R², together with the nitrogen to which they are attached, form any one of the following:

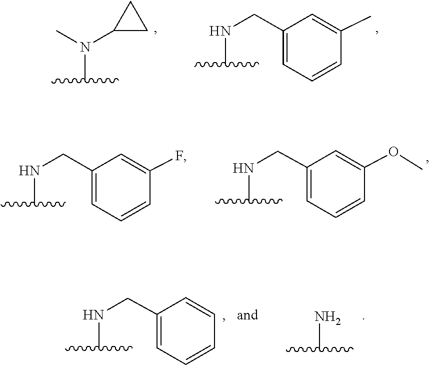

In some embodiments, R¹ and R², together with the nitrogen to which they are attached, form any one of the following:

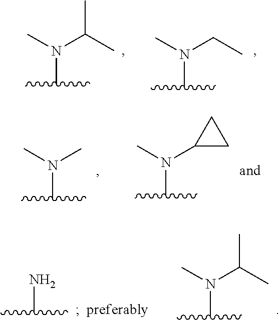

In some embodiments, R¹ and R² together with the nitrogen atom to which they are attached form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

### R³

In some embodiments, R³ is hydrogen.

In some embodiments, R³ and one of R¹ and R² together with the atoms to which they are attached to form a C₃₋₈ heterocycloalkyl, said C₃₋₈ heterocycloalkyl being further optionally substituted with a substituent selected from halogen, (O), CN, C₁₋₈ alkoxy, C₁₋₈ alkylamino, C₁₋₈alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

Z¹ and Z⁴

In some embodiments, Z¹ is CR⁸. In some embodiments, Z⁴ is CR¹¹. In some embodiments, Z¹ is CR⁸ and Z⁴ is CR¹¹.

In some embodiments, Z¹ is CR⁸, Z⁴ is CR¹¹ and R⁹ is not hydrogen. In some embodiments, Z¹ is CR⁸, Z⁴ is CR¹¹ and R⁹ is hydrogen.

In some embodiments, Z¹ is N. In some embodiments, Z⁴ is N. In some embodiments, Z¹ is CR⁸ and Z⁴ is N. In some embodiments, Z¹ is N and Z⁴ is CR¹¹.

In some embodiments, Z¹ is N and R⁹ is not hydrogen. In some embodiments, Z⁴ is N and R⁹ is not hydrogen. In some embodiments, Z¹ is CR⁸, Z⁴ is N and R⁹ is not hydrogen. In some embodiments, Z¹ is N, Z⁴ is CR¹¹ and R⁹ is not hydrogen.

In some embodiments, Z¹ is N and R¹ and R² are each independently selected from selected from hydrogen, C₁₋₆ alkyl, C₃₋₈ cycloalkyl, and C₇₋₁₈ alkylenearyl. In some embodiments, Z¹ is CR⁸ and R¹ and R² are each independently selected from selected from hydrogen, C₁₋₆ alkyl, C₃₋₈ cycloalkyl, and C₇₋₁₈ alkylenearyl. In some embodiments, Z⁴ is N and R¹ and R² are each independently selected from selected from hydrogen, C₁₋₆ alkyl, C₃₋₈ cycloalkyl, and C₇₋₁₈ alkylenearyl. In some embodiments, Z⁴ is CR¹¹ and R¹ and R² are each independently selected from selected from hydrogen, C₁₋₆ alkyl, C₃₋₈ cycloalkyl, and C₇₋₁₈ alkylenearyl.

R⁸, R⁹ and R¹¹

In some embodiments, one of R⁸, R⁹ and R¹¹ is hydrogen. In some embodiments, two of R⁸, R⁹ and R¹¹ are hydrogen. In some embodiments, all of R⁸, R⁹ and R¹¹ are hydrogen.

In some embodiments, at least one of R⁸, R⁹ and R¹¹ is halogen, preferably chloro or fluoro, more preferably fluoro. In some embodiments, one of R⁸, R⁹ and R¹¹ is halogen, preferably chloro or fluoro, more preferably fluoro. In some embodiments, R⁹ is halogen, preferably chloro or fluoro, more preferably fluoro.

In some embodiments, at least one of R⁸, R⁹ and R¹¹ is C₁₋₆ alkoxy, preferably C₁₋₄ alkoxy, more preferably methoxy. In some embodiments, one of R⁸, R⁹ and R¹¹ is C₁₋₆ alkoxy, preferably C₁₋₄ alkoxy, more preferably methoxy. In some embodiments, R⁸ is C₁₋₆ alkoxy, preferably C₁₋₄ alkoxy, more preferably methoxy. In some embodiments, R⁹ is C₁₋₆ alkoxy, preferably C₁₋₄ alkoxy, more preferably methoxy. In some embodiments, R¹¹ is C₁₋₆ alkoxy, preferably C₁₋₄ alkoxy, more preferably methoxy.

In some embodiments, at least one of R⁸, R⁹ and R¹¹ is OR¹³, preferably wherein R¹³ is hydrogen. In some embodiments, one of R⁸, R⁹ and R¹¹ is OR¹³, preferably wherein R¹³ is hydrogen. In some embodiments, R⁸ is OR¹³, preferably wherein R¹³ is hydrogen. In some embodiments, R⁹ is OR¹³, preferably wherein R¹³ is hydrogen. In some embodiments, R¹¹ is OR¹³, preferably wherein R¹³ is hydrogen.

In some embodiments, at least one of R⁸, R⁹ and R¹¹ is hydrogen and at least one of R⁸, R⁹ and R¹¹ is halogen (preferably chloro or fluoro, more preferably fluoro), C₁₋₆ alkoxy (preferably C₁₋₄ alkoxy, more preferably methoxy) or OR¹³ (preferably wherein R¹³ is hydrogen), In some embodiments, two of R⁸, R⁹ and R¹¹ are hydrogen the remaining one of R⁸, R⁹ and R¹¹ is halogen (preferably chloro or fluoro, more preferably fluoro), C₁₋₆ alkoxy (preferably C₁₋₄ alkoxy, more preferably methoxy) or OR¹³ (preferably wherein R¹³ is hydrogen), In some embodiments, R⁹ and R¹¹ are hydrogen and R⁸ is halogen (preferably chloro or fluoro, more preferably fluoro), C₁₋₆ alkoxy (preferably C₁₋₄ alkoxy, more preferably methoxy) or OR¹³ (preferably wherein R¹³ is hydrogen), In some embodiments, R⁸ and R¹¹ are hydrogen and R⁹ is halogen (preferably chloro or fluoro, more preferably fluoro), C₁₋₆ alkoxy (preferably C₁₋₄ alkoxy, more preferably methoxy) or OR¹³ (preferably wherein R¹³ is hydrogen). In some embodiments, R⁸ and R⁹ are hydrogen and R¹¹ is halogen (preferably chloro or fluoro, more preferably fluoro), C₁₋₆ alkoxy (preferably C₁₋₄ alkoxy, more preferably methoxy) or OR¹³ (preferably wherein R¹³ is hydrogen. In some embodiments, R⁹ and R¹¹ are hydrogen and R⁸ is OR¹³ (preferably wherein R¹³ is hydrogen).

In some embodiments, one of R⁸, R⁹ and R¹¹ is hydrogen and R¹ and R² are each independently selected from selected from hydrogen, C₁₋₆ alkyl, C₃₋₈ cycloalkyl, and C₇_₁₈ alkylenearyl. In some embodiments, two of R⁸, R⁹ and R¹¹ are hydrogen and R¹ and R² are each independently selected from selected from hydrogen, C₁₋₆ alkyl, C₃₋₈ cycloalkyl, and C₇₋₁₈ alkylenearyl. In some embodiments, all of R⁸, R⁹ and R¹¹ are hydrogen and R¹ and R² are each independently selected from selected from hydrogen, C₁₋₆ alkyl, C₃₋₈ cycloalkyl, and C₇₋₁₈ alkylenearyl.

In some embodiments, R⁸, R⁹ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O)R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any one of the foregoing paragraphs.

In some embodiments, one of two of R⁸, R⁹ and R¹¹ when present are each independently selected from halogen, CN, C₁₋₆ alkyl, C₁₋₆ haloalkyl and OR¹³ wherein R¹³ is selected from hydrogen, C₁₋₆ alkyl and C₁₋₆ haloalkyl, and the other of R⁸, R⁹ and R¹¹ are each hydrogen.

In some embodiments, one of R⁸, R⁹ and R¹¹ when present are each independently selected from halogen, CN, C₁₋₆ alkyl, C₁₋₆ haloalkyl and OR¹³ wherein R¹³ is selected from hydrogen, C₁₋₆ alkyl and C₁₋₆ haloalkyl, and the other of R⁸, R⁹ and R¹¹ are each hydrogen.

In some embodiments, one of R⁸, R⁹ and R¹¹ when present are each independently selected from halogen, CN, C₁₋₄ alkyl, C₁₋₄ haloalkyl and OR¹³ wherein R¹³ is selected from hydrogen, C₁₋₄ alkyl and C₁₋₄ haloalkyl, and the other of R⁸, R⁹ and R¹¹ are each hydrogen.

In some embodiments, one of R⁸, R⁹ and R¹¹ when present is fluoro, chloro, hydroxyl or OCH₃, and the other of R⁸, R⁹ and R¹¹ are each hydrogen.

In some embodiments, one of R⁸, R⁹ and R¹¹ when present is OCH₃, and the other of R⁸, R⁹ and R¹¹ are each hydrogen. In some embodiments, one of R⁸, R⁹ and R¹¹ when present is hydroxy, and the other of R⁸, R⁹ and R¹¹ are each hydrogen. In some embodiments, one of R⁸, R⁹ and R¹¹ when present is fluoro, and the other of R⁸, R⁹ and R¹ are each hydrogen. In some embodiments, one of R⁸, R⁹ and R¹¹ when present is chloro, and the other of R⁸, R⁹ and R¹¹ are each hydrogen.

In some embodiments, R⁸ is OCH₃, and R⁹ and R¹¹ (if present) are each hydrogen. In some embodiments, R⁸ is hydroxy, and R⁹ and R¹¹ (if present) are each hydrogen.

In some embodiments, R⁹ is OCH₃, and R⁸ and R¹¹ (if present) are each hydrogen. In some embodiments, R⁹ is hydroxy, and R⁸ and R¹¹ (if present) are each hydrogen. In some embodiments, R⁹ is fluoro, and R⁸ and R¹¹ (if present) are each hydrogen. In some embodiments, R⁹ is chloro, and R⁸ and R¹¹ (if present) are each hydrogen.

In some embodiments, R¹¹ is OCH₃, and R⁸ and R⁹ (if present) are each hydrogen. In some embodiments, R¹¹ is hydroxy, and R⁸ and R⁹ (if present) are each hydrogen.

### L

In some embodiments, L is C₁₋₄ alkylene.

In some embodiments, L is methylene.

### Further Formulas

In some embodiments, the compound of formula (I) has the formula (Ia):

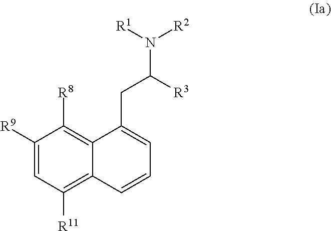

or a pharmaceutically acceptable salt, solvate, tautomer, N-oxide, stereoisomer, metabolite, polymorph and/or prodrug thereof,
wherein R¹, R², R³, R⁸, R⁹ and R¹¹ are as defined herein.

In some embodiments, the compound of formula (I) has the formula (Ib):

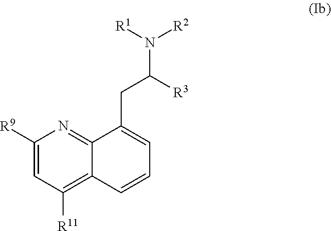

or a pharmaceutically acceptable salt, solvate, tautomer, N-oxide, stereoisomer, metabolite, polymorph and/or prodrug thereof,
wherein R¹, R², R³, R⁹ and R¹¹ are as defined herein.

In some embodiments, the compound of formula (I) has the formula (Ic):

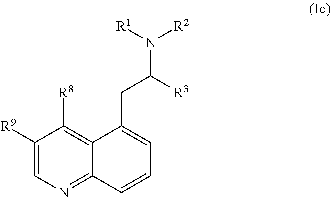

or a pharmaceutically acceptable salt, solvate, tautomer, N-oxide, stereoisomer, metabolite, polymorph and/or prodrug thereof,
wherein R¹, R², R³, R⁸ and R⁹ are as defined herein.

In some embodiments, the compound of formula (I) is selected from any one of the following:

| Compound No. | Structure |
|---|---|
| I-1 | 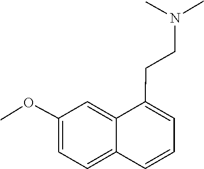 |
| I-2 | 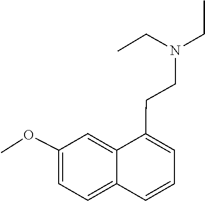 [SMILES: CCN(CC)CCc1cccc2ccc(OC)cc12] |
| I-3 | 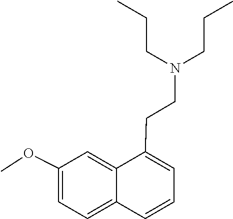 [SMILES: CCCN(CCC)CCc1cccc2ccc(OC)cc12] |
| I-4 | 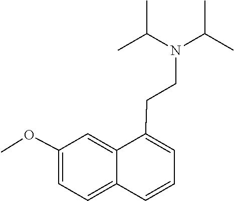 [SMILES: COc1ccc2cccc(CCN(C(C)C)C(C)C)c2c1] |
| I-5 | 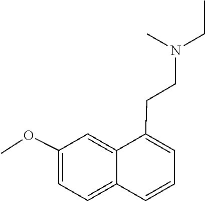 [SMILES: CCN(C)CCc1cccc2ccc(OC)cc12] |
| I-6 | 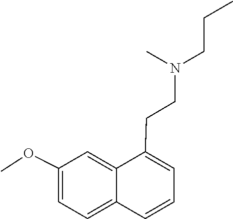 [SMILES: CCCN(C)CCc1cccc2ccc(OC)cc12] |
| I-7 | 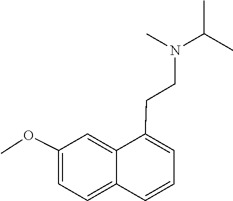 [SMILES: COc1ccc2cccc(CCN(C)C(C)C)c2c1] |
| I-14 | 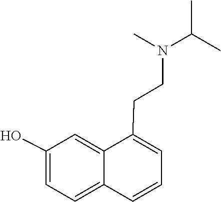 [SMILES: CC(C)N(C)CCc1cccc2ccc(O)cc12] |
| I-15 | 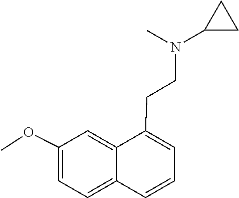 [SMILES: COc1ccc2cccc(CCN(C)C3CC3)c2c1] |
| I-16 | 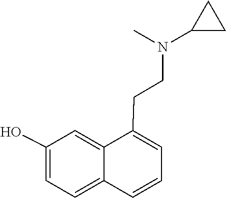 [SMILES: CN(CCc1cccc2ccc(O)cc12)C1CC1] |
| I-17 | 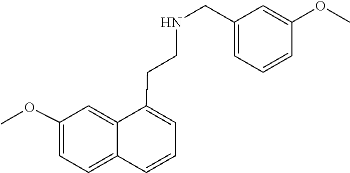 |
| I-18 | 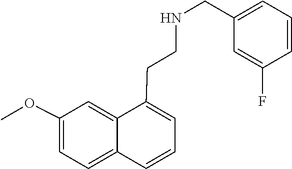 [SMILES: COc1ccc2cccc(CCNCc3cccc(F)c3)c2c1] |
| I-19 | 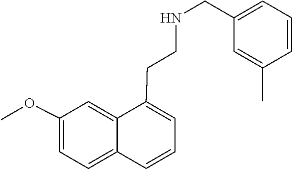 [SMILES: COc1ccc2cccc(CCNCc3cccc(C)c3)c2c1] |
| I-20 | 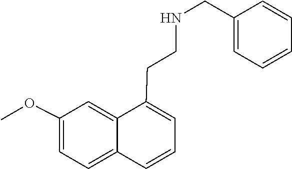 [SMILES: COc1ccc2cccc(CCNCc3ccccc3)c2c1] |
| I-13 | 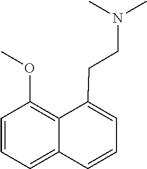 [SMILES: COc1cccc2cccc(CCN(C)C)c12] |
| I-21 | 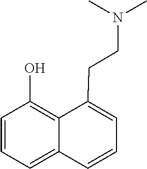 [SMILES: CN(C)CCc1cccc2cccc(O)c12] |
| I-22 | 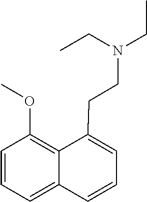 [SMILES: CCN(CC)CCc1cccc2cccc(OC)c12] |
| I-23 | 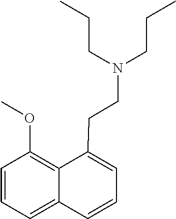 [SMILES: CCCN(CCC)CCc1cccc2cccc(OC)c12] |
| I-24 | 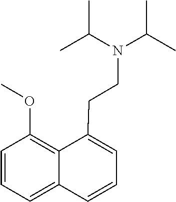 [SMILES: COc1cccc2cccc(CCN(C(C)C)C(C)C)c12] |
| I-25 | 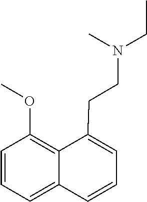 [SMILES: CCN(C)CCc1cccc2cccc(OC)c12] |
| I-26 | 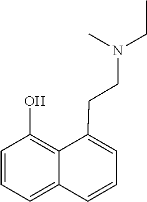 [SMILES: CCN(C)CCc1cccc2cccc(O)c12] |
| I-27 | 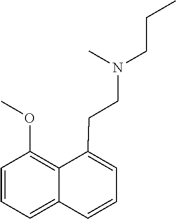 [SMILES: CCCN(C)CCc1cccc2cccc(OC)c12] |
| I-28 | 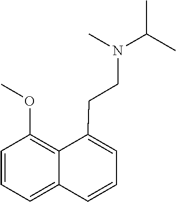 [SMILES: COc1cccc2cccc(CCN(C)C(C)C)c12] |
| I-29 | 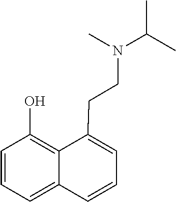 [SMILES: CC(C)N(C)CCc1cccc2cccc(O)c12] |
| I-30 | 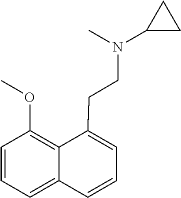 [SMILES: COc1cccc2cccc(CCN(C)C3CC3)c12] |
| I-31 | 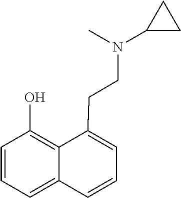 [SMILES: CN(CCc1cccc2cccc(O)c12)C1CC1] |
| I-32 | 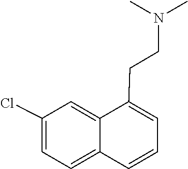 [SMILES: CN(C)CCc1cccc2ccc(Cl)cc12] |
| I-33 | 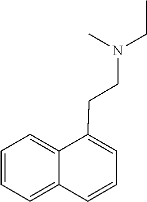 [SMILES: CCN(C)CCc1cccc2ccccc12] |
| I-34 | 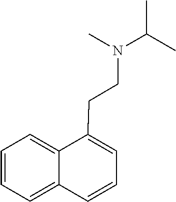 [SMILES: CC(C)N(C)CCc1cccc2ccccc12] |
| I-35 | 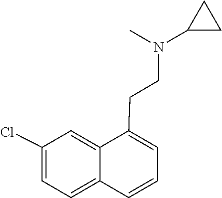 [SMILES: CN(CCc1cccc2ccc(Cl)cc12)C1CC1] |
| I-37 | 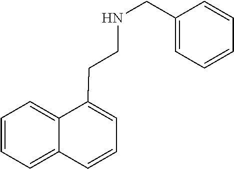 [SMILES: c1ccc(CNCCc2cccc3ccccc23)cc1] |
| I-53 | 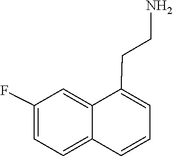 [SMILES: NCCc1cccc2ccc(F)cc12] |
| I-38 | 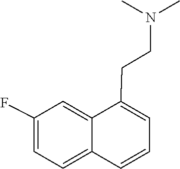 [SMILES: CN(C)CCc1cccc2ccc(F)cc12] |
| I-39 | 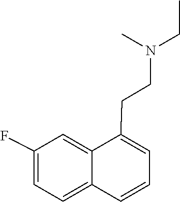 [SMILES: CCN(C)CCc1cccc2ccc(F)cc12] |
| I-40 | 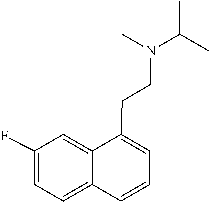 [SMILES: CC(C)N(C)CCc1cccc2ccc(F)cc12] |
| I-41 | 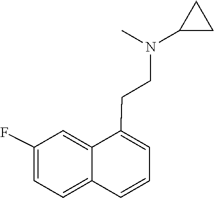 [SMILES: CN(CCc1cccc2ccc(F)cc12)C1CC1] |
| I-42 | 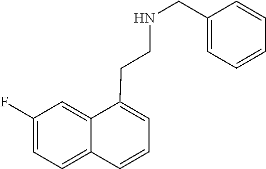 [SMILES: Fc1ccc2cccc(CCNCc3ccccc3)c2c1] |
| I-50 | 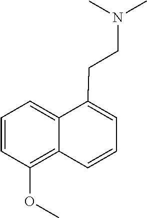 [SMILES: COc1cccc2c(CCN(C)C)cccc12] |
| I-52 | 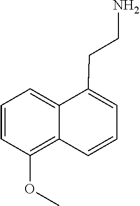 |
| I-8 | 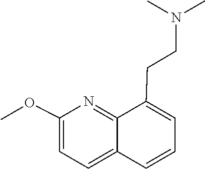 |
| I-9 | 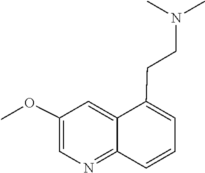 |

or a pharmaceutically acceptable salt, solvate, tautomer, N-oxide, stereoisomer, metabolite, polymorph and/or prodrug thereof.

In some embodiments, the compound of formula (I) is selected from any one of the following: I-1, I-2, I-3, I-4, I-5, I-6, I-7, I-13, I-8, and I-9, or a pharmaceutically acceptable salt, solvate, tautomer, N-oxide, stereoisomer, metabolite, polymorph and/or prodrug thereof.

In some embodiments, the compound of formula (I) is selected from any one of the following: I-1, I-5, I-7, I-13, I-8, and I-9, or a pharmaceutically acceptable salt, solvate, tautomer, N-oxide, stereoisomer, metabolite, polymorph and/or prodrug thereof.

In some embodiments, the compound of formula (I) is selected from any one of the following: I-1, I-5, I-7, I-13, and, I-8, or a pharmaceutically acceptable salt, solvate, tautomer, N-oxide, stereoisomer, metabolite, polymorph and/or prodrug thereof.

In some embodiments, the compound of formula (I) is selected from any one of the following: I-1, I-5, I-7, and, I-13, or a pharmaceutically acceptable salt, solvate, tautomer, N-oxide, stereoisomer, metabolite, polymorph and/or prodrug thereof.

In some embodiments, the compound of formula (I) is: I-8, or a pharmaceutically acceptable salt, solvate, tautomer, N-oxide, stereoisomer, metabolite, polymorph and/or prodrug thereof.

In some embodiments, the compound of formula (I) is: I-9, or a pharmaceutically acceptable salt, solvate, tautomer, N-oxide, stereoisomer, metabolite, polymorph and/or prodrug thereof.

In some embodiments the compound of formula (I) is selected from any one of the following: I-1, I-2, I-3, I-4, I-5, I-6, I-7, I-14, I-15, I-16, I-17, I-20, I-13, I-21, I-22, I-23, I-24, I-25, I-26, I-27, I-28, I-29, I-30, I-31, I-32, I-33, I-34, I-35, I-37, I-52, I-38, I-39, I-40, I-41, I-42, I-50, I-52, and I-53, or a pharmaceutically acceptable salt, solvate, tautomer, N-oxide, stereoisomer, metabolite, polymorph and/or prodrug thereof.

In some embodiments the compound of formula (I) is selected from any one of the following: I-14, I-15, I-16, I-17, I-18, I-19, I-20, I-21, I-22, I-23, I-24, I-25, I-26, I-27, I-28, I-29, I-30, I-31 I-32, I-33, I-34, I-35, I-37, I-52, I-38, I-39, I-40, I-41, I-42, I-50, I-52 and I-53, or a pharmaceutically acceptable salt, solvate, tautomer, N-oxide, stereoisomer, metabolite, polymorph and/or prodrug thereof.

### Forms of the Compound

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of Formula (I) including anhydrous crystalline forms, hydrates, solvates and mixed solvates. If any of these crystalline forms demonstrates polymorphism, all polymorphs are within the scope of this invention.

Formula (I) is intended to cover, where applicable, solvated as well as unsolvated forms of the compounds. Thus, Formula (I) includes compounds having the indicated structures, including the hydrated or solvated forms, as well as the non-hydrated and non-solvated forms.

The compounds of Formula (I) or salts, tautomers, N-oxides, polymorphs or prodrugs thereof may be provided in the form of solvates. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of the invention.

The compound of Formula (I) may demonstrate tautomerism. Tautomers are two interchangeable forms of a molecule that typically exist within an equilibrium. Any tautomers of the compounds of Formula (I) are to be understood as being within the scope of the invention.

The compound of Formula (I) may contain one or more stereocentres. All stereoisomers of the compounds of formula (I) are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (I) at any stereocentre. The compound may be enriched in one stereoisomer over another by at least about 60, 70, 80, 90, 95, 98 or 99%.

The compound of Formula (I) or its salts, tautomers, solvates, N-oxides, and/or stereoisomers, may be isotopically enriched with one or more of the isotopes of the atoms present in the compound. For example, the compound may be enriched with one or more of the following minor isotopes: ²H, ³H, ¹³C ¹⁴C, ¹⁵N and/or ¹⁷O, preferably ²H. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norylin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

### Compositions, Formulations and Modes of Administration

The compounds of formula (I) can be administered alone or in the form of a pharmaceutical composition. In practice, the compounds of formula (I) are usually administered in the form of pharmaceutical compositions, that is, in admixture with at least one pharmaceutically acceptable excipient. The proportion and nature of any pharmaceutically acceptable excipient(s) are determined by the properties of the selected compound of the invention, the chosen route of administration, and standard pharmaceutical practice.

In another embodiment, there is provided a pharmaceutical composition comprising a compound of formula (I) or a pharmaceutically acceptable salt, stereoisomer, solvate, metabolite, and/or polymorph thereof, and at least one pharmaceutically acceptable excipient.

Pharmaceutical compositions of the disclosure typically include a therapeutically effective amount of one or more active ingredients in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial perilymph, possibly supplemented with other materials common in compositions for parenteral administration. Neutral buffered saline or saline mixed with serum albumin are further exemplary vehicles.

Pharmaceutical compositions of the present disclosure additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminium hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present in the composition, according to the judgment of the formulator.

Various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

A compound of formula (I) may be administered in any form and route which makes the compound bioavailable.

Compositions described herein may be administered systemically or locally.

Compositions described herein may be formulated from compounds according to Formula (I) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions). In some embodiments, compositions described herein may be administered orally, nasally, intravenously, intramuscularly, topically, subcutaneously, rectally, vaginally or by urethral application.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A pharmaceutical composition may be formulated as inhaled formulations, including sprays, mists, or aerosols. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable Formulations, wherein the carrier is a liquid, for administration, as for example, a nasal spray or as nasal drops, include aqueous or oily solutions of the active ingredient.

Compositions suitable for rectal administration are preferably presented as unit dose suppositories. These may be prepared by at least partially dispersing the active in one or more lipophilic bases and then shaping the mixture.

Pharmaceutical compositions may be formulated as sustained release formulations such as a capsule that creates a slow release of active following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of active release. The amount of active contained within a sustained release formulation depends upon, for example, the site of implantation, the rate and expected duration of release and the nature of the condition to be treated.

One skilled in the art can readily select the proper form and route of administration depending on the particular characteristics of the compound selected, the disease or condition to be treated, the stage of the disease or condition, and other relevant circumstances.

In some embodiments, the pharmaceutical composition comprises a compound of formula (I) as described herein, or a pharmaceutically acceptable salt, solvate, tautomer, N-oxide, stereoisomer, metabolite, polymorph and/or prodrug thereof, an additional therapeutic agent, and a pharmaceutically acceptable excipient.

The additional agent may be any suitable agent described herein. In some embodiments, the additional agent is a psychoactive drug, including those described herein. In some embodiments, the additional agent is useful for treatment of a disease, disorder or condition ameliorated by activation of a serotonin receptor, including those described herein. In some embodiments, the additional agent is selected from any one of the following, including those described herein: an agent for a mental illness and/or a neuropsychiatric condition; an agent for psychosis and/or psychotic symptoms; an agent for attention deficit hyperactivity disorder and/or attention deficit disorder; an agent for dementia and/or Alzheimer's disease; and an agent for an addiction disorder.

### Methods and Uses

The present disclosure provides methods of using the compounds of formula (I) and compositions as described in any one of the foregoing paragraphs. The present disclosure also provides methods of delivering to a subject in need thereof a compound of formula (I) or a composition (e.g., an effective amount of the compound or composition) of the present disclosure.

In another aspect, the present disclosure provides methods of treating a disease in a subject in need thereof comprising administering to the subject in need thereof an effective amount (e.g., therapeutically effective amount) of a compound or composition (e.g., pharmaceutical composition) of the present disclosure.

In another aspect, the present disclosure provides methods of preventing a disease in a subject in need thereof comprising administering to the subject in need thereof an effective amount (e.g., therapeutically effective amount) of a compound of formula (I) or composition (e.g., pharmaceutical composition) of the present disclosure.

In another aspect, provided herein are uses of the compounds of formula (I) or compositions of the present disclosure in the manufacture of a medicament for use in any of the methods (e.g., method of delivering an active agent to a subject in need thereof, method of treating a disease in a subject in need thereof, method of preventing a disease in a subject in need thereof) of the present disclosure.

In another aspect, provided herein are uses of the compounds of formula (I) or compositions of the present disclosure in any of the methods (e.g., method of delivering an active agent to a subject in need thereof, method of treating a disease in a subject in need thereof, method of preventing a disease in a subject in need thereof) of the present disclosure.

In certain embodiments, the effective amount is effective in treating the disease. In certain embodiments, the effective amount is effective in preventing the disease.

In another aspect, the present disclosure provides a method of treating a disease, disorder or condition associated with activity of a serotonin receptor, the method comprising administering to a subject in need thereof a compound of formula (I) or a pharmaceutical composition as described herein.

In another aspect, the present disclosure provides the use of a compound of formula (I) as described herein in the manufacture of a medicament for treating a disease, disorder or condition associated with activity of a serotonin receptor.

In another aspect, the present disclosure provides the use of a compound of formula (I) or a pharmaceutical composition as described herein for treating a disease, disorder or condition associated with activity of a serotonin receptor.

In another aspect, the present disclosure provides a compound of formula (I) or a pharmaceutical composition as described herein for use in treating disease, disorder or condition associated with activity of a serotonin receptor.

In another aspect, the present disclosure provides a method of preventing a disease, disorder or condition associated with activity of a serotonin receptor, the method comprising administering to a subject in need thereof a compound of formula (I) or a pharmaceutical composition as described herein.

In another aspect, the present disclosure provides the use of a compound of formula (I) as described herein in the manufacture of a medicament for preventing a disease, disorder or condition associated with activity of a serotonin receptor.

In another aspect, the present disclosure provides the use of a compound of formula (I) or a pharmaceutical composition as described herein for preventing a disease, disorder or condition associated with activity of a serotonin receptor.

In another aspect, the present disclosure provides a compound of formula (I) or a pharmaceutical composition as described herein for use in preventing disease, disorder or condition associated with activity of a serotonin receptor.

In another aspect, the present disclosure provides method of treating a disease, disorder or condition associated with activity of a serotonin receptor, the method comprising administering to a subject in need thereof a compound of formula (I) or a pharmaceutical composition as described herein, in combination with another agent useful for treatment of said disease, disorder or condition. The other agent may be any agent useful for treating and/or preventing said disease, disorder or condition, including those known the art and described herein. The other agent may be a serotonin receptor agonist. The other agent may be another compound of formula (I). The other agent may be compound other than a compound of formula (I), including those known in the art and described herein.

In another aspect, the present disclosure provides the use of a compound of formula (I) as described herein in the manufacture of a medicament for treating a disease, disorder or condition associated with activity of a serotonin receptor, wherein the medicament is formulated for administration with another agent useful for treatment of said disease, disorder or condition as described herein.

In another aspect, the present disclosure provides the use of a compound of formula (I) or a pharmaceutical composition as described herein for treating a disease, disorder or condition associated with activity of a serotonin receptor, wherein the compound of formula (I) or pharmaceutical composition is formulated for administration with another agent useful for treatment of said disease, disorder or condition as described herein.

In another aspect, the present disclosure provides a compound of formula (I) or a pharmaceutical composition as described herein for use in treating disease, disorder or condition associated with activity of a serotonin receptor, wherein the compound of formula (I) or pharmaceutical composition, in use, is formulated for administration with another agent useful for treatment of said disease, disorder or condition as described herein.

In another aspect, the present disclosure provides method of preventing a disease, disorder or condition associated with activity of a serotonin receptor, the method comprising administering to a subject in need thereof a compound of formula (I) or a pharmaceutical composition as described herein, in combination with another agent useful for prevention of said disease, disorder or condition. The other agent may be any agent useful for treating and/or preventing said disease, disorder or condition, including those known the art and described herein. The other agent may be a serotonin receptor agonist. The other agent may be another compound of formula (I). The other agent may be compound other than a compound of formula (I), including those known in the art and described herein.

In another aspect, the present disclosure provides the use of a compound of formula (I) as described herein in the manufacture of a medicament for preventing a disease, disorder or condition associated with activity of a serotonin receptor, wherein the medicament is formulated for administration with another agent useful for treatment of said disease, disorder or condition as described herein.

In another aspect, the present disclosure provides the use of a compound of formula (I) or a pharmaceutical composition as described herein for preventing a disease, disorder or condition associated with activity of a serotonin receptor, wherein the compound of formula (I) or pharmaceutical composition is formulated for administration with another agent useful for treatment of said disease, disorder or condition as described herein.

In another aspect, the present disclosure provides a compound of formula (I) or a pharmaceutical composition as described herein for use in preventing disease, disorder or condition associated with activity of a serotonin receptor, wherein the compound of formula (I) or pharmaceutical composition, in use, is formulated for administration with another agent useful for treatment of said disease, disorder or condition as described herein.

In certain embodiments, the serotonin receptor is 5-HT_{2A}. In certain embodiments, the serotonin receptor is 5-HT_{2C}.

In certain embodiments, the serotonin receptor is one or both of 5-HT_{2A} and 5-HT_{2C}.

Additionally, or alternatively, in some embodiments, the serotonin receptor is not 5-HT_{2B}.

In some embodiments, the compound of formula (I) of the present disclosure is selective towards the 5-HT_{2A} receptor over one or both of the 5-HT_{2C} receptor and the 5-HT_{2B} receptor, preferably over the 5-HT_{2B} receptor. In some embodiments, the compound of formula (I) is selective towards the 5-HT_{2C} receptor over one or both of the 5-HT₂a receptor and the 5-HT_{2B} receptor, preferably over the 5-HT_{2B} receptor. In some embodiments, the compound of formula (I) is selective toward the 5-HT_{2A} receptor and 5-HT_{2C} receptor over the 5-HT_{2B} receptor.

In some embodiments, the compound of formula (I) of the present disclosure exhibits an EC₅₀ value for the 5-HT_{2A} receptor of less than about 1 mM, less than about 100 μM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2A} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2C} receptor of less than about 1 mM, less than about 100 μM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2C} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2B} receptor of greater than about 1 μM, greater than about 10 μM, or greater than about 100 μM, as determined by an assay described herein, for example an assay of calcium flux activity such as measuring changes in intracellular calcium.

In some embodiments, the present disclosure also includes a method of treating a mental illness or a neuropsychiatric condition comprising administering to a subject in need thereof a compound of formula (I) or a composition as described herein. The present disclosure also includes a use of a compound of formula (I) of the present disclosure for treatment of a mental illness or a neuropsychiatric condition, as well as a use of a compound of formula (I) of the present disclosure for the preparation of a medicament for treatment of a mental illness or a neuropsychiatric condition. The application further includes a compound of formula (I) of the present disclosure for use in treating a mental illness or a neuropsychiatric condition.

In some embodiments, the compound of formula (I) of the present disclosure is administered in combination with one or more additional agents for a mental illness or a neuropsychiatric condition. The one or more additional agents for a mental illness or a neuropsychiatric condition may be any suitable agents known in the art, including those described herein. In some embodiments, the additional agents for a mental illness or a neuropsychiatric condition is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; agents for an addiction disorder such as alcohol addiction (e.g., disulfiram), nicotine dependence (e.g., varenicline) and opioid use disorder (e.g., methadone, buprenorphine, buprenorphine-naloxone and buprenorphine long-acting injection); mood stabilizers such as lithium and anticonvulsants such carbamazepine, divalproex (valproic acid), lamotrigine, gabapentin and topiramate.

In some embodiments, the present disclosure also includes a method of treating neurodegeneration comprising administering to a subject in need thereof a compound of formula (I) or a composition as described herein. Also provided is a use of a compound of formula (I) of the present disclosure for treatment of neurodegeneration, as well as a use of a compound of formula (I) of the present disclosure for the preparation of a medicament for treatment neurodegeneration. The application further includes a compound of formula (I) of the present disclosure for use in treating neurodegeneration. In some embodiments, the disease, disorder or condition is reduced brain-derived neurotrophic factor (BDNF), mammalian target of rapamycin (mTOR) activation and/or inflammation.

In some embodiments, the disease, disorder or condition to be treated by the methods described herein comprise cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the present disclosure also includes a method of treating psychosis or psychotic symptoms comprising administering to a subject in need thereof a compound of formula (I) or a composition as described herein. The present disclosure also includes a use of a compound of formula (I) of the present disclosure for treatment of psychosis or psychotic symptoms, as well as a use of a compound of formula (I) of the present disclosure for the preparation of a medicament for treatment of psychosis or psychotic symptoms. The application further includes a compound of formula (I) of the present disclosure for use in treating psychosis or psychotic symptoms.

In some embodiments, the disease, disorder or condition that is to be treated by methods of the present disclosure is psychosis or psychotic symptoms and the the compound of formula (I) of the present disclosure is administered in combination with one or more additional agents for psychosis or psychotic symptoms. The one or more additional agents for psychosis or psychotic symptoms may be any suitable agents known in the art, including those described herein. In some embodiments, the additional agents for psychosis or psychotic symptoms are selected typical antipsychotics and atypical antipsychotics. The typical antipsychotics may be selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof. The atypical antipsychotics may be selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, administering to said subject in need thereof a therapeutically effective amount of the compound of formula (I) of the present disclosure does not result in a worsening of psychosis or psychotic symptoms such as, but not limited to, hallucinations and delusions. In some embodiments, administering to said subject in need thereof a therapeutically effective amount of the compound of formula (I) results in an improvement of psychosis or psychotic symptoms such as, but not limited to, hallucinations and delusions. In some embodiments, administering to said subject in need thereof a therapeutically effective amount of the compounds of formula (I) results in an improvement of psychosis or psychotic symptoms.

In some embodiments, the disease, disorder or condition that is to be treated by methods of the present disclosure is a central nervous system (CNS) disease, disorder or condition and/or a neurological disease, disorder or condition. Accordingly, the present disclosure also includes a method of treating a CNS disease, disorder or condition and/or a neurological disease, disorder or condition comprising administering a therapeutically effective amount of compound of formula (I) or a composition of the present disclosure to a subject in need thereof. The present disclosure also includes a use of compound of formula (I) of the present disclosure for treatment a CNS disease, disorder or condition and/or a neurological disease, disorder or condition, as well as a use of compound of formula (I) of the present disclosure for the preparation of a medicament for treatment of a CNS disease, disorder or condition and/or a neurological disease, disorder or condition. The application further includes a compound of formula (I) of the present disclosure of the application for use in treating a CNS disease, disorder or condition and/or a neurological disease, disorder or condition.

In some embodiments, the disease, disorder or condition that is to be treated by methods of the present disclosure is a central nervous system (CNS) disease, disorder or condition and/or a neurological disease, disorder or condition and the compound of formula (I) of the present disclosure is administered in combination with one or more additional agents for a central nervous system (CNS) disease, disorder or condition and/or a neurological disease, disorder or condition. The one or more additional agents for a central nervous system (CNS) disease, disorder or condition and/or a neurological disease, disorder or condition may be any suitable agents known in the art, including those described herein. In some embodiments, the additional agents for a central nervous system (CNS) disease, disorder or condition and/or a neurological disease, disorder or condition are selected from lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Non limiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline.

In some embodiments, the present disclosure also includes a method of treating attention deficit hyperactivity disorder and/or attention deficit disorder comprising administering to a subject in need thereof a compound of formula (I) or a composition as described herein. The present disclosure also includes a use of a compound of formula (I) of the present disclosure for treatment of attention deficit hyperactivity disorder and/or attention deficit disorder, as well as a use of a compound of formula (I) of the present disclosure for the preparation of a medicament for treatment of attention deficit hyperactivity disorder and/or attention deficit disorder. The application further includes a compound of formula (I) of the present disclosure for use in treating attention deficit hyperactivity disorder and/or attention deficit disorder.

In some embodiments, the disease, disorder or condition to be treated by methods of the present disclosure is attention deficit hyperactivity disorder and/or attention deficit disorder and a combination thereof and the compound of formula (I) of the present disclosure is administered in combination with one or more additional agents for attention deficit hyperactivity disorder and/or attention deficit disorder and a combination thereof. The one or more additional agents for attention deficit hyperactivity disorder and/or attention deficit disorder may be any suitable agents known in the art, including those described herein. In some embodiments, the additional agents for attention deficit hyperactivity disorder and/or attention deficit disorder and a combination thereof are selected from methylphenidate, dexamphetamine, lisdexamfetine, atomoxetine and amphetamine and a combination thereof.

In some embodiments, the disease, disorder or condition that is to be treated by methods of the present disclosure is selected from dementia and Alzheimer's disease and a combination thereof. Accordingly, the present disclosure also includes a method of treating dementia and/or Alzheimer's disease comprising administering to a subject in need thereof a compound of formula (I) or a composition as described herein. The present disclosure also includes a use of a compound of formula (I) of the present disclosure for treatment of dementia and/or Alzheimer's disease, as well as a use of a compound of formula (I) of the present disclosure for the preparation of a medicament for treatment of dementia and/or Alzheimer's disease. The application further includes a compound of formula (I) of the present disclosure for use in treating dementia and/or Alzheimer's disease.

In some embodiments, the disease, disorder or condition that is to be treated by the methods of the present disclosure is dementia or Alzheimer's disease and the compound of formula (I) of the present disclosure is administered in combination with one or more additional agents for dementia or Alzheimer's disease. The one or more additional agents for dementia or Alzheimer's disease may be any suitable agents known in the art, including those described herein. In some embodiments, the additional agents for dementia and Alzheimer's disease are selected from acetylcholinesterase inhibitors, NMDA antagonists and nicotinic agonists. The acetylcholinesterase inhibitors may be selected from donepezil, galantamine, rivastigmine, and phenserine, and combinations thereof. The NMDA antagonists may be selected from MK-801, ketamine, phencyclidine, and memantine, and combinations thereof. The nicotinic agonists may be selected from nicotine, nicotinic acid, nicotinic alpha7 agonists, or alpha2 beta4 agonists or a combination thereof.

In another aspect, the present disclosure provides a method of treating a mental illness, the method comprising administering to a subject in need thereof a compound of formula (I) or a pharmaceutical composition as described herein. In another aspect, the present disclosure provides a method of preventing a mental illness, the method comprising administering to a subject in need thereof a compound of formula (I) or a pharmaceutical composition as described herein. The mental illness may be a neuropsychiatric condition.

In certain embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, mania, schizophrenia, schizoaffective disorder, schizophreniform disorder; impulse control and addiction disorders, such as pyromania (starting fires), kleptomania (stealing) and compulsive gambling; alcohol addiction; drug addiction, such as opioid addiction/dependence, nicotine dependence, cocaine dependence, marijuana abuse and so on; personality disorders, such as antisocial personality disorder, aggression, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilias; somatic symptom disorders, formerly known as a psychosomatic disorder or somatoform disorder.

In certain embodiments, the mental illness is selected from hallucinations and delusions and a combination thereof. In these embodiments, the hallucinations may be selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In another aspect, the present disclosure provides a method for treating a central nervous system (CNS) disease, disorder or condition and/or a neurological disease, disorder or condition, the method comprising administering to a subject in need thereof a compound of formula (I) or a pharmaceutical composition as described herein.

In another aspect, the present disclosure provides a method for preventing a central nervous system (CNS) disease, disorder or condition and/or a neurological disease, disorder or condition, the method comprising administering to a subject in need thereof a compound of formula (I) or a pharmaceutical composition as described herein.

In some embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; Tic disorder; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect, the present disclosure provides a method for increasing neuronal plasticity, the method comprising contacting a neuronal cell with a compound of formula (I) or a pharmaceutical composition as described herein, in an amount sufficient to increase neuronal plasticity of the neuronal cell. “Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapses in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention; weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect, the present disclosure provides a method for increasing dendritic spine density, the method comprising contacting a neuronal cell with a compound of formula (I) or a pharmaceutical composition as described herein, in an amount sufficient to increase dendritic spine density of the neuronal cell.

In certain embodiments, the compound of formula (I) produces a maximum number of dendritic crossings with an increase of greater than 1.0 fold by a Sholl Analysis.

In another aspect the present disclosure provides a method for activating a serotonin receptor in a cell, either in a biological sample or in a patient, comprising administering a compound of formula (I) as defined herein to the cell. The serotonin receptor may be a 5-HT receptor subtype, preferably one or both of 5-HT_{2A} and 5-HT_{2C}. In some embodiments, the compound of formula (I) is selective for one or both of 5-HT_{2A} and 5-HT_{2C}.

It will be understood, that the specific dose level for any particular patient will depend upon a variety of factors including the activity of the specific compound employed, the age, body weight, general health, sex, diet, time of administration, route of administration, number of doses, and rate of excretion, drug combination (i.e. other drugs being used to treat the patient), and the severity of the particular disorder undergoing therapy.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

As used herein, the term “effective amount” means that amount of a drug or pharmaceutical agent that will elicit the biological or medical response of a tissue, system, animal or human that is being sought, for instance, by a researcher or clinician. Furthermore, the phrase “therapeutically effective amount” generally refers to an amount of one or more active ingredients of the invention that (i) treats the particular disease, condition, or disorder, (ii) attenuates, ameliorates, or eliminates one or more sign or symptoms of the particular disease, condition, or disorder, or (iii) delays the onset of one or more sign or symptoms of the particular disease, condition, or disorder described herein.

Typically, a therapeutically effective dosage is formulated to contain a concentration (by weight) of at least about 0.1% up to about 50% or more, and all combinations and sub-combinations of ranges therein. The compositions can be formulated to contain one or more actives described herein in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

In some embodiments, the compounds of formula (I) of the present disclosure are administered one, two, three or four times a year. In some embodiments, the compounds of the present disclosure are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. The treatment period may be for the duration of the detectable disease. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered at doses that are not hallucinogenic or psychotomimetic. In some preferred embodiments, therapeutically effective amounts of a compound of formula (I) described herein are not hallucinogenic or psychotomimetic. Sub-hallucinogenic or psychotomimetic doses may be administered to the subject once daily, once every two days, once every 3 days, once a week, once every two weeks, once a month, once every two months, or once every three months.

A compound of formula (I) of the present disclosure may be either used alone or in combination with other agents useful for treating the diseases, disorders or conditions to be treated by methods of the present disclosure, including diseases, disorders and conditions ameliorated by activation of a serotonin receptor, such as the compounds of the present disclosure. The other agent may be any agent useful for treating said disease, disorder or condition, including those known the art and described herein. The other agent may be a serotonin receptor agonist. In some embodiments, the other agent is another compound of formula (I). In some embodiments, the other agent is compound other than a compound of formula (I), including those known in the art and described herein. When used in combination with other agents, it is an embodiment that a compound of formula (I) is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present disclosure that a combination of agents is administered to a subject in a non-contemporaneous fashion. In some embodiments, a compound of formula (I) of the present disclosure is administered with another therapeutic agent simultaneously or sequentially in separate unit dosage forms or together in a single unit dosage form. Accordingly, the present disclosure provides a single unit dosage form comprising one or more compounds of formula (I) as described herein, an additional therapeutic agent and a pharmaceutically acceptable carrier. However, it will be appreciated, that when administered in separate dosage forms the route of administration may be the same or different.

In some embodiments, the compounds of formula (I) as described herein are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

### Kits

In another embodiment there is provided a kit or article of manufacture including one or more compounds, pharmaceutically acceptable salt, stereoisomer, solvate, metabolite, or polymorph, and/or pharmaceutical compositions as described above.

In other embodiments there is provided a kit for use in a therapeutic application mentioned above, the kit including:
a container holding one or more compounds, pharmaceutically acceptable salt, stereoisomer, solvate, metabolite, and/or polymorph, and/or pharmaceutical compositions as described herein;
a label or package insert with instructions for use. The instructions may be according to any of the methods or uses described herein.

It will be understood that the invention disclosed and defined in this specification extends to all alternative combinations of two or more of the individual features mentioned or evident from the text or drawings. All of these different combinations constitute various alternative aspects of the invention.

### EXAMPLES

Reference will now be made to specific embodiments of the invention. While the synthetic protocols outlined below will describe specific embodiments of the invention, it is understood that the intention is not to limit the invention to those embodiments. On the contrary, the invention is intended to cover all alternatives, modifications, and equivalents, which may be included within the scope of the present invention and defined by the claims.

One skilled in the art will recognise numerous methods and materials similar or equivalent to those described herein. The present invention is in no way limited to the materials and methods described. It will be understood that the choice of structural features or substitution patterns surrounding the core scaffolds outlined above will influence the selection of one process over another. Starting materials are available from commercial sources or may be readily prepared from available precursors following straightforward transformations that are well known to one skilled in the art.

### General

In the examples below, unless otherwise stated, temperatures are given in degrees Celsius (° C.); operations were carried out at room or ambient temperature, “rt,” or “RT,” (typically a range of from about 18-25° C.; evaporation of solvent was carried out using a rotary evaporator under reduced pressure (typically, 4.5-30 mm Hg) with a bath temperature of up to 60° C.; the course of reactions was typically followed by thin layer chromatography (TLC); melting points are uncorrected; products exhibited satisfactory ¹H NMR and/or microanalytical data; and the following conventional abbreviations are also used: L (litres), mL (millilitres), mmol (millimoles), g (grams), mg (milligrams), min (minutes), and h (hours).

Unless otherwise specified, all solvents and reagents were purchased from suppliers and used without further purification. Reactions were conducted under a blanket of nitrogen unless otherwise stated. Compounds were visualized under UV lamp (254 nm). ¹H NMR spectra were recorded on a 300 MHz or 400 MHz NMR instrument as indicated. Column and flash chromatography was performed using SiO₂ as the stationary phase and “MeOH/NH₃” refers to a 9:1 solution of methanol to 15M ammonia aqueous.

### Synthesis of Compounds

### General Procedures

General Procedure A: Formulation of Hydrochloride Salts from Amines Starting freebase amine is dissolved in a minimal amount of solvent (MeOH, iPrOH or mixture thereof) and acidified to pH 1 by addition of concentrated HCl (32-37%). Precipitation is initiated by addition of Et₂O and the mixture is left to stand at 0° C. The product is collected by vacuum filtration and washed with Et₂O.
General Procedure B: Formulation of Fumaric Acid Salts from Amines

A solution of freebase amine in a minimal amount of solvent (acetone or iPrOH) is added to a hot solution of fumaric acid in either acetone or iPrOH (I-3 eq., 0.02-0.2 M) and the mixture is heated to between 40-60° C. The mixture is cooled and precipitation is initiated by addition of Et₂O or hexane and then left to stand at 0° C. The product is collected by vacuum filtration and washed with Et₂O.

Compounds of general formula (I) can be synthesised from an appropriately substituted 6,6-aromatic system following the steps outlined in Schemes 1-9 below or similar as one skilled in the art may consider. Various substituted 6,6-aromatic systems are commercially available or may be prepared by techniques known in the art, for example as described in WO2015/158313 and Landagaray E et al (European Journal of Medicinal Chemistry, Volume 127, 15 Feb. 2017, Pages 621-631).

Scheme 1: Compounds of general formula (I) can be synthesised from an appropriately substituted 6,6-aromatic system following the outlined sequence of steps in Scheme 1 or similar as one skilled in the art may utilise. An appropriately substituted 6,6-aromatic system can undergo sequential one-pot reductive alkylation(s) to access compounds of general formula (I) (exemplified by Example I-1 and I-18).

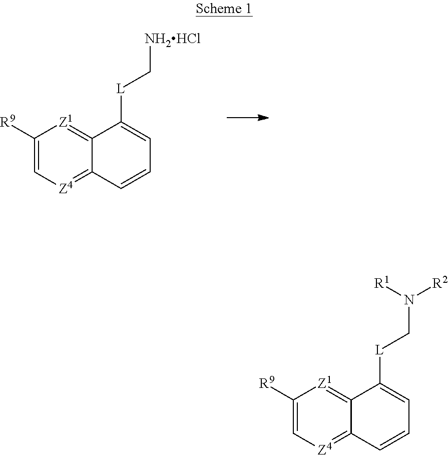

### Example 1: Synthesis of 2-(7-methoxynaphthalen-1-yl)-N,N-dimethylethan-1-amine (I-1)

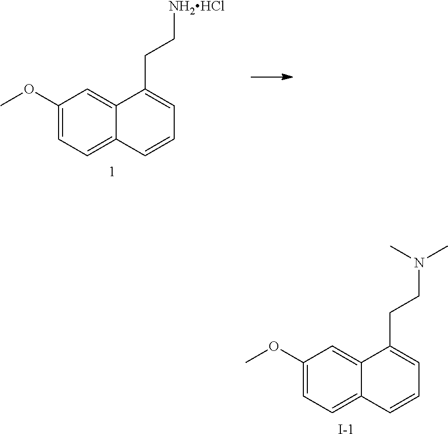

### Step 1: 2-(7-methoxynaphthalen-1-yl)-N,N-dimethylethan-1-amine (I-1)

A solution of 2-(7-methoxynaphthalen-1-yl)ethan-1-amine hydrochloride (0.3 g, 1.26 mmol) in MeOH (6 mL) was treated with AcOH (303 mg, 5.05 mmol), NaBH₃CN (159 mg, 2.52 mmol), and 37% (w/w) aq. formaldehyde (102 mg, 1.26 mmol) at 0° C. The reaction was then stirred at RT for 24 h. Upon completion, the pH was adjusted to 8 with saturated aq. Na₂CO₃ and then extracted with CH₂Cl₂ (8 mL×3). The combined organic layer was washed with brine (5 mL), dried (Na₂SO₄), filtered, and concentrated in vacuo. The crude product was purified by preparative HPLC (column: Phenomenex C18 80*40 mm*3 μm; mobile phase: [H₂O (NH₄HCO₃)-ACN]; B %: 10%-40%, 8 min) to afford 2-(7-methoxynaphthalen-1-yl)-N,N-dimethylethan-1-amine (I-1, 52 mg, 18%) as an off-white solid. HPLC purity: 98.4% (220 nm); LCMS (ESI+) m/z 230.1 [M+H]⁺; ¹H NMR (400 MHz, MeOD-d₄) δ 7.76 (d, J=9.2 Hz, 1H), 7.65 (d, J=8.0 Hz, 1H), 7.32-7.35 (m, 2H), 7.24-7.26 (m, 1H), 7.14 (d, J=2.4 Hz, 1H), 3.95 (s, 3H), 3.21-3.25 (m, 2H), 2.65-2.69 (m, 2H), 2.41 (s, 6H).

Scheme 2: Compounds of general formula (I) can be synthesised from an appropriately substituted 6,6-aromatic system following the outlined sequence of steps in Scheme 2 or similar as one skilled in the art may utilise. A 6,6-aromatic system with a suitable nitrile substituent can be selectively reduced to aldehyde intermediates that can subsequently undergo reductive alkylation with appropriately substituted amines to generate compounds of general formula (I) (exemplified by Examples I-2, I-3, I-4, I-5, I-6, and I-7).

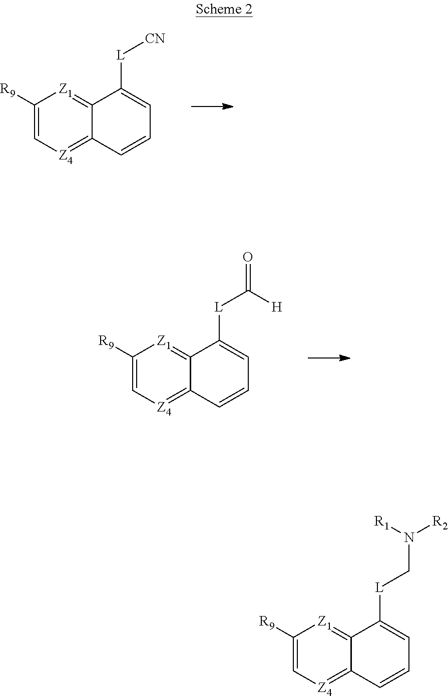

### Example 2: Synthesis of N,N-diethyl-2-(7-methoxynaphthalen-1-yl)ethan-1-amine (I-2)

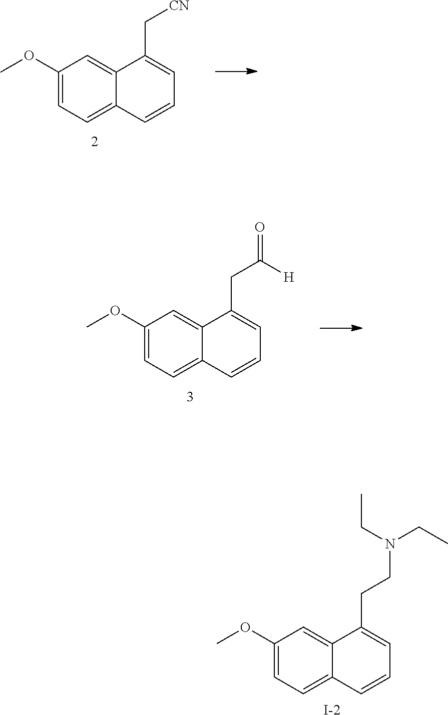

### Step 1: 2-(7-methoxynaphthalen-1-yl)acetaldehyde (3)

A solution of 2-(7-methoxynaphthalen-1-yl)acetonitrile (0.3 g, 1.52 mmol) in CH₂Cl₂ (10 mL) was treated with 1 M DiBAL-H in toluene (2.28 mL, 2.28 mmol) dropwise at 0° C. and the resulting mixture was stirred at 0° C. for 3 h. Upon completion, the reaction was quenched with 3 M aq. HCl (30 mL) and extracted with CH₂Cl₂ (15 mL×3). The combined organic layer was washed with brine (20 mL×2), dried over anhydrous Na₂SO₄, filtered, and concentrated in vacuo. The resulting residue was purified by flash chromatography (10% EtOAc in petroleum ether) to afford 2-(7-methoxynaphthalen-1-yl)acetaldehyde (164 mg, 54%) as a yellow oil. ¹H NMR (300 MHz, CDCl₃) δ 9.74 (t, J=2.1 Hz, 1H), 7.78 (m, 2H), 7.31-7.42 (m, 2H), 7.18 (dd, J=9.0, 2.1 Hz, 1H), 7.11 (s, 1H), 4.05 (d, J=2.1 Hz, 2H), 3.92 (s, 3H)

### Step 2: N,N-diethyl-2-(7-methoxynaphthalen-1-yl)ethan-1-amine hydrochloride (I-2·HCl)

A solution of 2-(7-methoxynaphthalen-1-yl)acetaldehyde (80 mg, 0.4 mmol), iPr₂NEt (78 mg, 0.6 mmol), and N,N-diethylamine (44 mg, 0.6 mmol) in CH₂Cl₂ (3 mL) was treated with NaBH(OAc)₃ (212 mg, 1.0 mmol) in portions at RT and the resulting mixture was stirred at RT for 16 h. Upon completion, the reaction was quenched with H₂O (20 mL) and extracted with CH₂Cl₂ (15 mL×3). The combined organic layer was washed with brine (20 mL×2), dried (Na₂SO₄), filtered, and concentrated in vacuo. The residue was purified by preparative TLC to afford N,N-diethyl-2-(7-methoxynaphthalen-1-yl)ethan-1-amine (40 mg) as the free base (I-2). The free base was dissolved in MeOH (0.5 mL) and treated with HCl/Et₂O at RT and stirred at RT for 30 min. The reaction mixture was concentrated in vacuo and the residue was triturated with Et₂O. The solid was collected to afford the hydrochloride salt (I-2·HCl, 20 mg, 17%). HPLC purity: 98.9% (254 nm); LCMS (ESI+) m/z 258.4 [M+H]⁺; ¹H NMR (300 MHz, CD₃OD-d₄) δ 7.82 (d, J=8.1 Hz, 1H), 7.75 (d, J=8.1 Hz, 1H), 7.40 (d, J=6.9 Hz, 1H), 7.35-7.25 (m, 2H), 7.19 (d, J=9.0 Hz, 1H), 3.98 (s, 3H), 3.56-3.35 (m, 8H), 1.39 (t, J=6.6 Hz, 6H).

### Example 3: Synthesis of N-(2-(7-methoxynaphthalen-1-yl)ethyl)-N-propylpropan-1-amine (I-3)

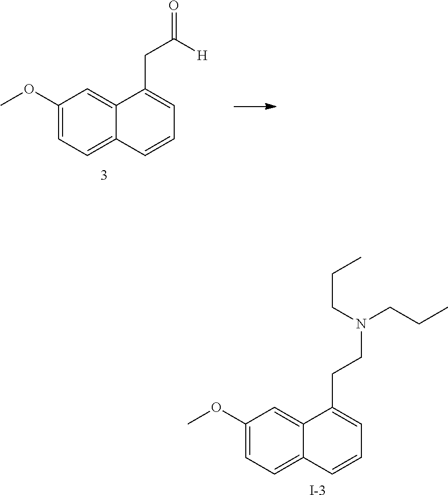

Step 1: N-(2-(7-methoxynaphthalen-1-yl)ethyl)-N-propylpropan-1-amine (I-3) A solution of 2-(7-methoxynaphthalen-1-yl)acetaldehyde (0.1 g, 0.50 mmol), iPr₂NEt (97 mg, 0.75 mmol), and N,N-dipropylamine (76 mg, 0.75 mmol) in CH₂Cl₂ (5 mL) was treated with NaBH(OAc)₃ (265 mg, 1.25 mmol) in portions at RT and the resulting mixture was stirred at RT for 16 h. Upon completion, the reaction was quenched with H₂O (10 mL) and extracted with CH₂Cl₂ (10 mL×3). The combined organic layer was dried over anhydrous Na₂SO₄, filtered, and concentrated in vacuo. The residue was purified by preparative TLC to afford the title compound (I-3, 20 mg, 14%) as a white solid. HPLC purity: 99.9% (254 nm); LCMS (ESI+) m/z 286.4 [M+H]⁺; ¹H NMR (300 MHz, CD₃OD-d₄) δ 7.81 (d, J=8.1 Hz, 1H), 7.74 (d, J=8.1 Hz, 1H), 7.44 (d, J=6.9 Hz, 1H), 7.38-7.25 (m, 2H), 7.19 (d, J=6.9 Hz, 1H), 3.98 (s, 3H), 3.58-3.42 (m, 4H), 3.30-3.18 (m, 4H), 1.91-1.73 (m, 4H), 1.04 (t, J=7.2 Hz, 6H).

### Example 4: Synthesis of N-isopropyl-N-(2-(7-methoxynaphthalen-1-yl)ethyl)propan-2-amine (I-4)

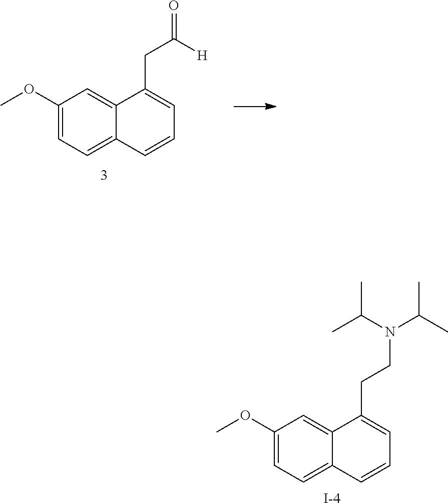

### Step 1: N-Isopropyl-N-(2-(7-methoxynaphthalen-1-yl)ethyl)propan-2-amine (I-4)

A solution of 2-(7-methoxynaphthalen-1-yl)acetaldehyde (0.1 g, 0.50 mmol), iPr₂NEt (97 mg 0.75 mmol), and N,N-diisopropylamine (76 mg 0.75 mmol) in CH₂Cl₂ (5 mL) was treated with NaBH(OAc)₃ (265 mg 1.25 mmol) in portions at RT and the resulting mixture was stirred at RT for 16 h. Upon completion, the reaction was quenched with H₂O (20 mL) and extracted with CH₂Cl₂ (15 mL×3). The combined organic layer was washed with brine (20 mL×2), dried over anhydrous Na₂SO₄, filtered, and concentrated in vacuo. The residue was purified by preparative TLC to afford the title compound (I-4, 20 mg, 14%) as a white solid. HPLC purity: 99.8% (254 nm); LCMS (ESI+): m/z 286.3 [M+H]⁺; ¹H NMR (300 MHz, CD₃OD-d₄) δ 7.82 (d, J=8.1 Hz, 1H), 7.75 (d, J=8.1 Hz, 1H), 7.45 (d, J=6.9 Hz, 1H), 7.38-7.28 (m, 2H), 7.20 (dd, J=6.9, 2.1 Hz, 1H), 3.97 (s, 3H), 3.92-3.82 (m, 2H), 3.60-3.50 (m, 2H), 3.50-3.35 (m, 2H), 1.60-1.40 (m, 12H).

### Example 5: Synthesis of N-ethyl-2-(7-methoxynaphthalen-1-yl)-N-methylethan-1-amine (I-5)

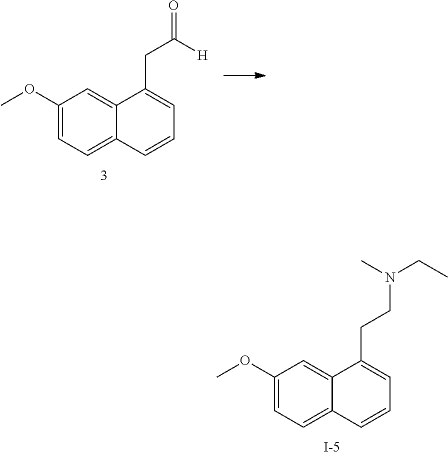

### Step 1: N-ethyl-2-(7-methoxynaphthalen-1-yl)-N-methylethan-1-amine (I-5·HCl)

A solution of 2-(7-methoxynaphthalen-1-yl)acetaldehyde (0.1 g, 0.5 mmol), iPr₂NEt (97 mg, 0.75 mmol), and N-ethyl-N-methylamine (44 mg, 0.75 mmol) in CH₂Cl₂ (5 mL) was treated with NaBH(OAc)₃ (265 mg, 1.25 mmol) in portions at RT and the resulting mixture was stirred at RT for 16 h. Upon completion, the reaction was quenched with H₂O (20 mL) and extracted with CH₂Cl₂ (15 mL×3). The combined organic layers were washed with brine (20 mL×2) dried over anhydrous Na₂SO₄, filtered, and concentrated in vacuo. The residue was purified by preparative TLC to afford the title compound as the free base (I-5, 60 mg) which was a colourless oil. The free base was dissolved in MeOH (0.5 mL) and treated with HCl/Et₂O at RT and stirred at RT for 30 min. The reaction mixture was concentrated in vacuo and the residue was triturated with Et₂O. The solid was collected to afford N-ethyl-2-(7-methoxynaphthalen-1-yl)-N-methylethan-1-amine hydrochloride (I-5·HCl, 20 mg, 14%). HPLC purity: 98.0% (254 nm); LCMS (ESI+): m/z 244.3 [M+H]⁺; ¹H NMR (300 MHz, CD₃OD-d₄) δ 7.78 (d, J=7.5 Hz, 1H), 7.71 (d, J=7.2 Hz, 1H), 7.48-7.32 (m, 2H), 7.31-7.22 (m, 1H), 7.15 (d, J=8.7 Hz, 1H), 3.98 (s, 3H), 3.65-3.32 (m, 6H), 2.98 (s, 3H), 1.38 (t, J=6.0 Hz, 3H).

### Example 6: Synthesis of N-(2-(7-methoxynaphthalen-1-yl)ethyl)-N-methylpropan-1-amine (I-6)

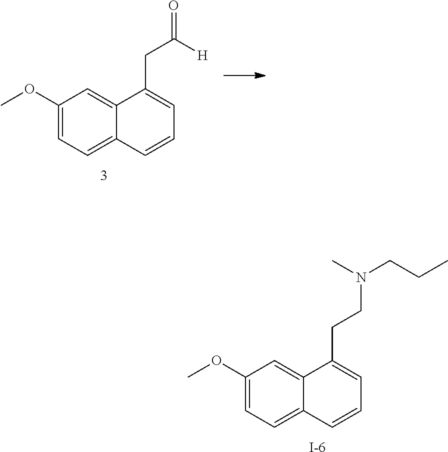

### Step 1: N-(2-(7-Methoxynaphthalen-1-yl)ethyl)-N-methylpropan-1-amine (I-6·HCl)

A solution of 2-(7-methoxynaphthalen-1-yl)acetaldehyde (70 mg, 0.35 mmol), iPr₂NEt (68 mg, 1.5 eq., 0.52 mmol), and N-methyl-N-propylamine (39 mg, 0.52 mmol) in CH₂Cl₂ (5 mL) was treated with NaBH(OAc)₃ (185 mg, 0.87 mmol) in portions at RT and the resulting mixture was stirred at RT for 16 h. Upon completion, the reaction was quenched with H₂O (30 mL) and extracted with CH₂Cl₂ (10 mL×3). The combined organic layer was washed with brine (15 mL×2) dried over anhydrous Na₂SO₄, filtered, and concentrated in vacuo. The residue was purified by preparative TLC to afford the title compound as the free base (I-6, 85 mg) which was a colourless oil. The free base was dissolved in MeOH (0.5 mL) and treated with HCl/Et₂O and stirred at RT for 30 minutes. The reaction mixture was concentrated in vacuo and the residue was triturated with Et₂O. The solid was collected to afford N-(2-(7-Methoxynaphthalen-1-yl)ethyl)-N-methylpropan-1-amine hydrochloride (I-6·HCl, 20 mg, 19%). HPLC purity: 99.6% (254 nm); LCMS (ESI+): m/z 258.4 [M+H]⁺; ¹H NMR (300 MHz, CD₃OD-d₄) δ 7.81 (d, J=8.1 Hz, 1H), 7.75 (d, J=8.1 Hz, 1H), 7.43 (d, J=6.9 Hz, 1H), 7.38-7.28 (m, 2H), 7.19 (d, J=6.6 Hz, 1H), 3.99 (s, 3H), 3.60-3.40 (m, 4H), 3.29-3.11 (m, 2H), 3.02 (s, 3H), 1.95-1.68 (m, 2H), 1.04 (t, J=7.2 Hz, 3H).

### Example 7: Synthesis of N-(2-(7-methoxynaphthalen-1-yl)ethyl)-N-methylpropan-2-amine (I-7)

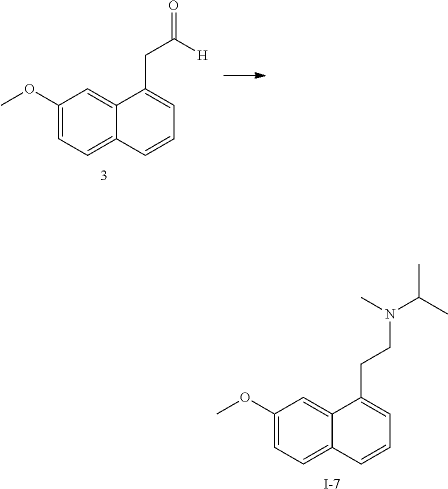

### Step 1: N-(2-(7-methoxynaphthalen-1-yl)ethyl)-N-methylpropan-2-amine (I-7)

A solution of 2-(7-methoxynaphthalen-1-yl)acetaldehyde (0.1 g, 0.5 mmol), iPr₂NEt (97 mg, 0.75 mmol), and N-isopropyl-N-methylamine (55 mg, 0.75 mmol) in CH₂Cl₂ (5 mL) was treated with NaBH(OAc)₃ (265 mg, 1.25 mmol) in portions at RT and the resulting mixture was stirred at RT for 16 h. Upon completion, the reaction was quenched with H₂O (10 mL) and extracted with CH₂Cl₂ (10 mL×3). The combined organic layer was dried over anhydrous Na₂SO₄, filtered, and concentrated in vacuo. The residue was purified by preparative TLC to afford the title compound as the free base (I-7, 50 mg) which was a colourless oil. The free base was dissolved in MeOH (0.5 mL) and treated with HCl/Et₂O and the resultant suspension stirred at RT for 30 min. The reaction mixture was concentrated in vacuo and the residue was triturated with Et₂O. The solid was collected to afford N-(2-(7-methoxynaphthalen-1-yl)ethyl)-N-methylpropan-2-amine hydrochloride (I-7·HCl, 20 mg, 16%). HPLC purity: 99.7% (254 nm); LCMS (ESI+): m/z 258.3 [M+H]⁺; ¹H NMR (300 MHz, CD₃OD-d₄) δ 7.82 (d, J=8.1 Hz, 1H), 7.75 (d, J=8.1 Hz, 1H), 7.44 (d, J=6.3 Hz, 1H), 7.40-7.28 (m, 2H), 7.20 (d, J=9.0 Hz, 1H), 3.98 (s, 3H), 3.88-3.72 (m, 1H), 3.60-3.40 (m, 4H), 2.95 (s, 3H), 1.40 (d, J=6.6 Hz, 3H), 1.34 (d, J=6.3 Hz, 3H).

### Example 14: Synthesis of 8-(2-(isopropyl(methyl)amino)ethyl)naphthalen-2-ol (I-14)

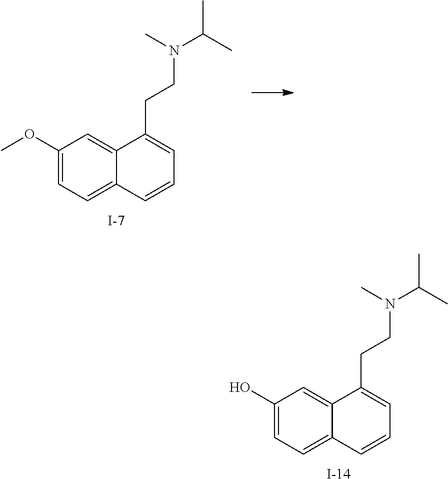

### Step 1: 8-(2-(isopropyl(methyl)amino)ethyl)naphthalen-2-ol (I-14)

To an ice-cold solution of N-(2-(7-methoxynaphthalen-1-yl)ethyl)-N-methylpropan-2-amine (620 mg, 2.41 mmol) in anhydrous CH₂Cl₂ (30 mL) was added BBr₃ (0.57 mL, 2.5 eq., 6.02 mmol) and the mixture was stirred at 0° C. for 1.5 h. The reaction was quenched with dropwise addition of 6 M aq. HCl until effervescence ceased upon addition. A further 1 mL of 6 M aq. HCl was added, followed by 5 mL of MeOH, and the resulting solution was refluxed for 1 h. The solvent was then removed under a stream of N₂ gas, and the aqueous residue was neutralised with saturated aq. Na₂CO₃. The suspension was made basic with 15% aq. NaOH (1 mL) and then extracted with CH₂Cl₂ (50 mL×3). The combined organic layer was washed with saturated aq. Na₂CO₃ (50 mL×2), then brine (50 mL), and then dried over anhydrous Na₂SO₄, filtered, and the filtrate concentrated under a stream of N₂ gas. The residue was purified by flash chromatography (0.1% to 5% MeOH/NH₃ in CH₂Cl₂) to afford the title compound as a light brown oil (480 mg, 82%). ¹H NMR (400 MHz, CDCl₃) δ 7.72 (d, J=8.8 Hz, 1H), 7.62 (d, J=7.9 Hz, 1H), 7.52 (d, J=2.1 Hz, 1H), 7.25-7.12 (m, 3H), 6.64 (br. s, 1H), 3.38-3.24 (m, 2H), 3.19 (sept, J=6.6 Hz, 1H), 3.00-2.85 (m, 2H), 2.50 (s, 3H), 1.15 (d, J=6.6 Hz, 6H). ¹³C NMR (101 MHz, CDCl₃) δ 155.77, 133.43, 133.40, 130.71, 128.91, 127.20, 126.99, 122.66, 118.97, 105.97, 54.45, 54.40, 36.15, 30.82, 17.52.

### Step 2: 8-(2-(isopropyl(methyl)amino)ethyl)naphthalen-2-ol hydrochloride (I-14·HCl)

8-(2-(isopropyl(methyl)amino)ethyl)naphthalen-2-ol (380 mg, 1.56 mmol) was formulated as the hydrochloride salt according to general procedure A which was isolated as pink crystals (116 mg, 31%). ¹H NMR (400 MHz, DMSO-d₆): δ 10.49 (br s, 1H), 9.90 (s, 1H), 7.79 (d, J=8.8 Hz, 1H), 7.73-7.68 (m, 1H), 7.40-7.33 (m, 2H), 7.22 (dd, J=8.2, 7.0 Hz, 1H), 7.14 (dd, J=8.8, 2.3 Hz, 1H), 3.66 (sept, J=6.6 Hz, 1H), 3.46-3.36 (m, 2H), 3.28-3.11 (m, 2H), 2.79 (s, 3H), 1.34-1.20 (m, 6H); ¹³C NMR (101 MHz, DMSO-d₆): δ 155.9, 132.9, 131.1, 130.3, 128.0, 127.2, 126.9, 122.2, 118.4, 105.0, 56.1, 52.3, 34.5, 27.2, 16.9, 15.2.

Scheme 3: Compounds of general formula (I) can be synthesised from an appropriately substituted 6,6-aromatic system following the outlined sequence of steps in Scheme 3 or similar as one skilled in the art may utilise. A 6,6-aromatic system with a suitable carboxylic acid substituent can be activated in numerous methods including, but not limited to, conversion to the acyl chloride, a mixed anhydride, an activated ester requiring the use of peptide coupling reagents, an activated urea requiring the use of imide coupling reagents, to be followed by amidation with the appropriately substituted amine. The resulting amides are able to undergo reduction with various reducing agents as one skilled in the art may consider resulting in the formation of compounds of general formula (I) (exemplified by Examples I-15, I-17, I-19, and I-20).

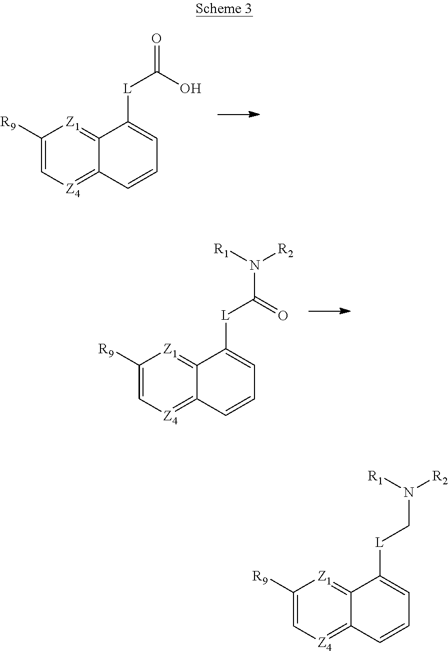

### Example 15: Synthesis of N-(2-(7-methoxynaphthalen-1-yl)ethyl)-N-methylcyclopropanamine (I-15)

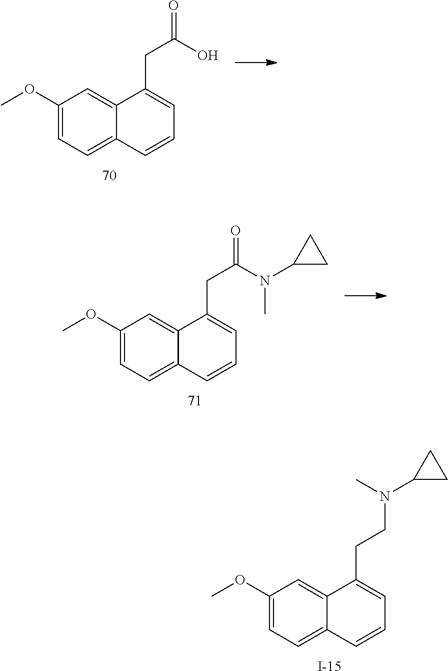

### Step 1: N-cyclopropyl-2-(7-methoxynaphthalen-1-yl)-N-methylacetamide (71)

To an ice-cold solution of (7-methoxy-1-naphthyl)acetic acid (500 mg, 2.31 mmol) in toluene (10 mL) was added thionyl chloride (0.34 mL, 4.62 mmol) and stirred at 60° C. for 1 h. The reaction mixture was then concentrated under a stream of N₂ gas overnight to give a solid. The solid was suspended in CH₂Cl₂ (10 mL) and treated with N-methyl(cyclopropyl)amine hydrochloride (373 mg, 3.47 mmol) and then iPr₂NEt (2.0 mL, 5 equiv., 11.6 mmol) and stirred at RT for 30 mins. The reaction was then diluted with 20 mL of CH₂Cl₂ and 0.1 M aq. HCl was added until the aqueous layer was acidic. The layers were separated, and the organic layer was washed with saturated aq. NaHCO₃ (20 mL) and brine (50 mL). The organic layer was dried over anhydrous Na₂SO₄, filtered, and the filtrate concentrated in vacuo. The residue was purified by flash chromatography (0.1% to 3% MeOH/NH₃ in CH₂Cl₂) to afford the title compound as an off-white solid (571 mg, 92%). ¹H NMR (400 MHz, DMSO-d₆) δ 7.84 (d, J=9.4 Hz, 1H), 7.77-7.68 (m, 1H), 7.34-7.23 (m, 2H), 7.22-7.14 (m, 2H), 4.24 (s, 2H), 3.86 (s, 3H), 2.95-2.76 (m, 4H), 0.97-0.79 (m, 4H). ¹³C NMR (101 MHz, DMSO-d₆) δ 172.7, 157.2, 133.3, 131.8, 130.0, 128.7, 128.0, 126.7, 123.0, 117.6, 103.2, 55.1, 38.3, 33.6, 31.3, 8.8.

### Step 2: N-(2-(7-methoxynaphthalen-1-yl)ethyl)-N-methylcyclopropanamine (I-15)

To ice-cold anhydrous THF (50 mL) was added LiAlH₄ (590 mg, 15.4 mmol) in portions, followed by a solution of N-cyclopropyl-N-methyl(7-methoxy-1-naphthyl)acetamide (520 mg, 1.93 mmol), in anhydrous THF (5 mL). The reaction was then stirred at reflux for 16 h under a N₂ atmosphere. The reaction was then cooled to 0° C. and quenched by sequential addition of H₂O (0.6 mL), NaOH (0.6 mL, 15%, w/v aq. solution), H₂O (1.8 mL) before being dried (Na₂SO₄) and filtered through a celite plug. The residue washed with hot THF (50 mL×2). The combined filtrates were concentrated, and the residue was purified by flash chromatography (0.1% to 2% MeOH/NH₃ in CH₂Cl₂) to afford the title compound as a clear oil (400 mg, 81%). ¹H NMR (400 MHz, CDCl₃) δ 7.75 (d, J=8.9 Hz, 1H), 7.65 (d, J=8.0 Hz, 1H), 7.40-7.22 (m, 3H), 7.15 (dd, J=8.9, 2.5 Hz, 1H), 3.94 (s, 3H), 3.35-3.20 (m, 2H), 3.01-2.81 (m, 2H), 2.53 (s, 3H), 1.86-1.75 (m, 1H), 0.54 (d, J=6.5 Hz, 4H). ¹³C NMR (101 MHz, CDCl₃) δ 157.9, 135.3, 133.2, 130.4, 129.4, 127.0, 126.7, 123.4, 118.2, 102.4, 58.9, 55.4, 42.9, 38.6, 31.1, 6.9.

### Step 3: N-(2-(7-methoxynaphthalen-1-yl)ethyl)-N-methylcyclopropanamine fumarate (I-15·fum)

A solution of N-cyclopropyl-N-methyl[2-(7-methoxy-1-naphthyl)ethyl]amine (365 mg, 1.43 mmol) dissolved in minimal anhydrous Et₂O was added to a solution of fumaric acid (166 mg, 1.43 mmol) dissolved in minimal refluxing acetone. The resultant solution was allowed to cool to 4° C. and maintained at this temperature for 72 h to afford a white crystalline solid that was identified as the fumarate salt of the title compound. (149 mg, 28%). ¹H NMR (400 MHz, DMSO-d₆) δ 7.83 (d, J=9.0 Hz, 1H), 7.70 (d, J=8.1 Hz, 1H), 7.37-7.30 (m, 2H), 7.26 (dd, J=8.0, 7.0 Hz, 1H), 7.17 (dd, J=8.9, 2.5 Hz, 1H), 6.61 (s, 2H), 3.90 (s, 3H), 3.29-3.11 (m, 2H), 2.93-2.78 (m, 2H), 2.48 (s, 3H), 1.98-1.85 (m, 1H), 0.56-0.46 (m, 2H), 0.43-0.33 (m, 2H). ¹³C NMR (101 MHz, DMSO-d₆) δ 166.2, 157.3, 134.9, 134.1, 132.6, 130.2, 128.8, 126.9, 126.3, 123.2, 117.9, 102.2, 57.8, 55.0, 41.9, 37.9, 29.8, 6.3; ¹H qNMR Purity: 97.2% (ERETIC); LCMS (ESI+) m/z 256.1 ([M+H]⁺).

### Example 16: Synthesis of 8-(2-(cyclopropyl(methyl)amino)ethyl)naphthalen-2-ol (I-16)

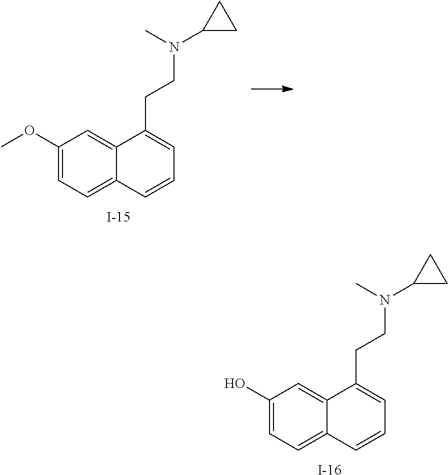

### Step 1: 8-(2-(cyclopropyl(methyl)amino)ethyl)naphthalen-2-ol (I-16·fum)

To an ice-cold solution of N-(2-(7-methoxynaphthalen-1-yl)ethyl)-N-methylcyclopropanamine (250 mg, 0.98 mmol) in anhydrous CH₂Cl₂ (15 mL) was added BBr₃ (0.19 mL, 1.96 mmol) and the mixture was stirred at 0° C. for 1.5 h. The reaction was diluted with CH₂Cl₂ (20 mL) and washed with saturated aq. Na₂CO₃ (10 mL×2) and then brine (10 mL). The organic was evaporated under a stream of N₂ gas and the residue was purified by column chromatography (0.1% to 2% MeOH/NH₃ in CH₂Cl₂) to afford the product as a borane complex. This complex was dissolved in 5 mL of MeOH and 1 mL of 6 M aq. HCl and heated to reflux for 1 h. The solvent was removed under a stream of N₂ gas to afford the title compound as a yellow oil which was used directly for fumarate formation using General Procedure B to afford the title compound as the fumarate salt (90 mg, 26%) which was an off-white solid. HPLC purity: 99.8% (220 nm); ¹H NMR (400 MHz, DMSO-d₆): δ 7.75 (d, J=8.8 Hz, 1H), 7.63 (d, J=8.0 Hz, 1H), 7.27 (dd, J=7.1, 1.6 Hz, 2H), 7.17 (dd, J=8.1, 6.9 Hz, 1H), 7.07 (dd, J=8.8, 2.3 Hz, 1H), 6.62 (s, 4H), 3.18-3.07 (m, 2H), 2.90-2.80 (m, 2H), 2.49 (s, 3H), 1.99-1.91 (m, 1H), 0.59-0.39 (m, 4H). ¹³C NMR (101 MHz, DMSO-d₆): δ 166.2, 155.5, 134.1, 133.9, 133.0, 130.2, 128.0, 126.6, 126.4, 122.3, 118.1, 105.0, 57.9, 42.1, 38.0, 29.8, 6.2.

### Example 17: Synthesis of N-(3-methoxybenzyl)-2-(7-methoxynaphthalen-1-yl)ethan-1-amine (I-17)

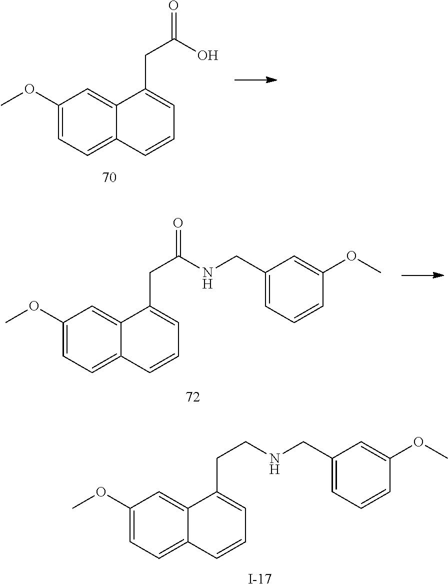

### Step 1: N-(3-methoxybenzyl)-2-(7-methoxynaphthalen-1-yl)acetamide (72)

To a solution of 2-(7-methoxynaphthalen-1-yl)acetic acid (0.30 g, 1.39 mmol) in DMF (2.0 mL) at 0° C. was added a solution of 2-(3H-[1,2,3]triazolo[4,5-b]pyridin-3-yl)-1,1,3,3-tetramethylisouronium hexafluorophosphate (1.06 g, 2.77 mmol), ⁱPr₂NEt (0.72 g, 5.55 mmol) and then (3-methoxyphenyl)methanamine (190 mg, 1.39 mmol) and the mixture was stirred at RT for 3 h. The reaction was diluted with H₂O (30 mL) and then extracted with EtOAc (3×20 mL). The combined organics were washed with brine (20 mL), dried over anhydrous MgSO₄ and concentrated under reduced pressure. The crude material was purified by flash chromatography (0-40% EtOAc/Hexane) to afford N-(3-methoxybenzyl)-2-(7-methoxynaphthalen-1-yl)acetamide as white crystals (403 mg, 87%). ¹H NMR (400 MHz, CDCl₃) δ 7.77 (d, J=8.9 Hz, 1H), 7.75 (d, J=7.8 Hz, 1H), 7.38 (dd, J=7.0, 1.3 Hz, 1H), 7.30 (dd, J=8.2, 7.0 Hz, 1H), 7.22-7.16 (m, 2H), 7.10 (t, J=7.9 Hz, 1H), 6.74-6.68 (m, 1H), 6.59 (ddd, J=7.6, 1.7, 0.9 Hz, 1H), 6.51 (t, J=2.1 Hz, 1H), 5.65 (t, J=5.9 Hz, 1H), 4.33 (d, J=6.0 Hz, 2H), 4.05 (s, 2H), 3.86 (s, 3H), 3.63 (s, 3H).

### Step 2: N-(3-methoxybenzyl)-2-(7-methoxynaphthalen-1-yl)ethan-1-amine (I-17)

To a stirred solution of N-(3-methoxybenzyl)-2-(7-methoxynaphthalen-1-yl)acetamide (379 mg, 1.13 mmol) in anhydrous THF (10 mL) was added 2 M borane-dimethylsulfide complex in THF (2.3 mL, 4.52 mmol) dropwise and the mixture was stirred at 60° C. for 1 h. After cooling, the mixture was carefully treated with 6 M aq. HCl (2.0 mL) followed by MeOH (4.0 mL) and then stirred at reflux for 1 h. After cooling, volatiles were removed under a stream of N₂ gas and the remaining aqueous phase extracted with Et₂O (2×10 mL) which was then discarded. The pH of the aqueous layer was adjusted to 14 with NaOH (1 M aq. soln.) and then extracted with Et₂O (3×10 mL). The combined organics were washed with brine (15 mL), dried over anhydrous MgSO₄ and concentrated under reduced pressure. The crude material was purified by flash chromatography (2% to 10% MeOH/NH₃ in CH₂Cl₂) to afford N-(3-methoxybenzyl)-2-(7-methoxynaphthalen-1-yl)ethan-1-amine as a clear oil (274 mg, 74%). ¹H NMR (400 MHz, CDCl₃) δ 7.75 (d, J=8.9 Hz, 1H), 7.66 (d, J=8.0 Hz, 1H), 7.36-7.11 (m, 5H), 6.91-6.84 (m, 2H), 6.82-6.73 (m, 1H), 3.90 (s, 3H), 3.83 (s, 2H), 3.77 (s, 3H), 3.27 (t, J=7.3 Hz, 2H), 3.02-3.10 (t, J=7.3 Hz, 2H).

### Step 3: N-(3-methoxybenzyl)-2-(7-methoxynaphthalen-1-yl)ethan-1-amine fumarate (I-17·Fum)

N-(3-methoxybenzyl)-2-(7-methoxynaphthalen-1-yl)ethan-1-amine (274 mg, 0.85 mmol) was formulated as the fumarate salt according to general procedure B which was isolated as white crystals (296 mg, 79%). HPLC purity: 95.4% (254 nm). ¹H NMR (400 MHz, DMSO-d₆) δ 7.84 (d, J=9.0 Hz, 1H), 7.77-7.69 (m, 1H), 7.36 (d, J=2.6 Hz, 1H), 7.35-7.32 (m, 1H), 7.27 (dd, J=8.1, 7.2 Hz, 2H), 7.18 (dd, J=8.9, 2.5 Hz, 1H), 7.07 (dd, J=2.6, 1.5 Hz, 1H), 7.04-6.97 (m, 1H), 6.88 (ddd, J=8.3, 2.6, 1.0 Hz, 1H), 6.55 (s, 2H), 4.01 (s, 2H), 3.88 (s, 3H), 3.74 (s, 3H), 3.36-3.29 (m, 2H), 3.07-2.98 (m, 2H). ¹³C NMR (101 MHz, DMSO-d₆) δ 167.3, 159.3, 157.6, 137.3, 134.8, 133.4, 132.6, 130.2, 129.5, 128.8, 127.2, 126.8, 123.2, 121.2, 117.9, 114.4, 113.4, 102.4, 55.3, 55.0, 50.8, 47.6, 30.7.

### Example 18: Synthesis of N-(3-fluorobenzyl)-2-(7-methoxynaphthalen-1-yl)ethan-1-amine (I-18)

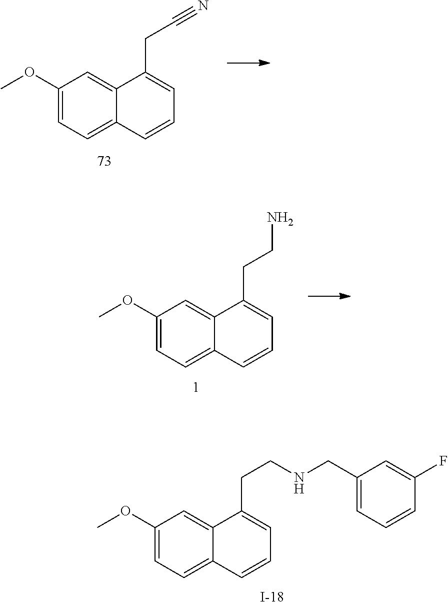

### Step 1: 2-(7-methoxynaphthalen-1-yl)ethan-1-amine (1)

To a solution of 2-(7-methoxynaphthalen-1-yl)acetonitrile (1.09 g, 5.07 mmol) in anhydrous THF (10 mL) was added 2 M borane-dimethylsulfide complex in THF (10.1 mL, 20.3 mmol) and the mixture was stirred at 80° C. for 4 h. After cooling, the mixture was carefully treated with 6 M aq. HCl (4.0 mL) followed by MeOH (8.0 mL) and then stirred at reflux for 1 h. After cooling, volatiles were removed under a stream of nitrogen and the remaining aqueous phase was extracted with Et₂O (2×10 mL) which was then discarded. The pH was adjusted to 14 with 5 M aq. NaOH and then extracted with Et₂O (3×10 mL). The combined organics were washed with brine (15 mL), dried over anhydrous MgSO₄ and concentrated under reduced pressure. The crude material was purified by flash chromatography (SiO₂, 2-10% MeOH/NH₃ in CH₂Cl₂) to afford 2-(7-methoxynaphthalen-1-yl)ethan-1-amine as a clear oil (877 mg, 86%). ¹H NMR (400 MHz, CDCl₃) δ 7.76 (d, J=8.9 Hz, 1H), 7.67 (dt, J=8.0, 1.1 Hz, 1H), 7.34-7.25 (m, 3H), 7.16 (dd, J=8.9, 2.5 Hz, 1H), 3.94 (s, 3H), 3.23-3.17 (m, 2H), 3.16-3.09 (m, 2H).

### Step 1: N-(3-fluorobenzyl)-2-(7-methoxynaphthalen-1-yl)ethan-1-amine (I-18)

To a stirred solution of 2-(7-methoxynaphthalen-1-yl)ethan-1-amine (92 mg, 0.46 mmol) in CH₂Cl₂ (5.0 mL) was added 3-fluorobenzaldehyde (57 mg, 0.46 mmol) and NaBH(OAc)₃ (116 mg, 0.55 mmol) and the reaction was left to stir at room temperature for 3 h. The reaction mixture was concentrated under a stream of nitrogen gas and then treated with 1 M aq. NaOH (3.0 mL) and extracted with EtOAc (3×5.0 mL). The combined organics were washed with brine, dried over anhydrous MgSO₄ and concentrated under reduced pressure. The crude material was purified by flash chromatography (SiO₂, 1% MeOH/NH₃ in CH₂Cl₂) to provide N-(3-fluorobenzyl)-2-(7-methoxynaphthalen-1-yl)ethan-1-amine as a clear oil (86 mg, 61%). ¹H NMR (400 MHz, DMSO-d₆) δ 7.76 (d, J=8.9 Hz, 1H), 7.69-7.65 (m, 1H), 7.33 (dd, J=7.0, 1.4 Hz, 1H), 7.30 (d, J=2.5 Hz, 1H), 7.29-7.22 (m, 2H), 7.16 (dd, J=8.9, 2.5 Hz, 1H), 7.07-7.01 (m, 2H), 6.96-6.89 (m, 1H), 3.91 (s, 3H), 3.83 (s, 2H), 3.31-3.22 (m, 2H), 3.09-3.01 (m, 2H).

### Example 19: Synthesis of N-(3-methylbenzyl)-2-(7-methoxynaphthalen-1-yl)ethan-1-amine (I-19)

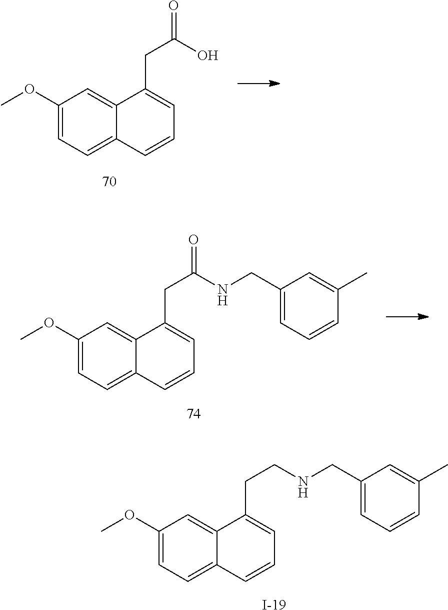

### Step 1: N-(3-methylbenzyl)-2-(7-methoxynaphthalen-1-yl)acetamide (74)

To a solution of 2-(7-methoxynaphthalen-1-yl)acetic acid (0.30 g, 1.39 mmol) in DMF (2.0 mL) at 0° C. was added a solution of 2-(3H-[1,2,3]triazolo[4,5-b]pyridin-3-yl)-1,1,3,3-tetramethylisouronium hexafluorophosphate (1.06 g, 2.77 mmol), iPr₂NEt (0.72 g, 5.55 mmol) and then m-tolylmethanamine (168 mg, 1.39 mmol) and the mixture was stirred at room temperature for 3 h. The reaction was diluted with H₂O (30 mL) and then extracted with EtOAc (3×20 mL). The combined organics were washed with brine (20 mL), dried over anhydrous MgSO₄ and concentrated under reduced pressure. The crude material was purified by flash chromatography (SiO₂, 0-40% EtOAc/Hexane) to afford 2-(7-methoxynaphthalen-1-yl)-N-(3-methylbenzyl)acetamide as a white crystals (348 mg, 79%). ¹H NMR (400 MHz, CDCl₃) δ 7.78 (d, J=8.7 Hz, 1H), 7.75 (d, J=8.3 Hz, 1H), 7.38 (dd, J=7.3, 1.1 Hz, 1H), 7.30 (dd, J=8.1, 7.0 Hz, 1H), 7.22-7.16 (m, 2H), 7.07 (t, J=7.5 Hz, 1H), 6.97 (d, J=7.6 Hz, 1H), 6.80 (d, J=7.4 Hz, 1H), 6.74-6.71 (m, 1H), 5.62 (t, J=6.1 Hz, 1H), 4.32 (d, J=6.0 Hz, 2H), 4.05 (s, 2H), 3.85 (s, 3H), 2.18 (s, 3H).

### Step 2: N-(3-methylbenzyl)-2-(7-methoxynaphthalen-1-yl)ethan-1-amine (I-19)

To a solution of 2-(7-methoxynaphthalen-1-yl)-N-(3-methylbenzyl)acetamide (320 mg, 1.00 mmol) in anhydrous THF (10 mL) was added 2 M borane-dimethylsulfide complex in THF (2.0 mL, 4.01 mmol) dropwise and the mixture was stirred at 60° C. for 1 h. After cooling, the mixture was carefully treated with 6 M aq. HCl (2.0 mL) followed by MeOH (4.0 mL) and then stirred at reflux for 1 h. After cooling, volatiles were removed under a stream of nitrogen and the remaining aqueous phase was extracted with Et₂O (2×10 mL) which was then discarded. The pH was adjusted to 14 with 5 M aq. NaOH and then extracted with Et₂O (3×10 mL). The combined organics were washed with brine (15 mL), dried over anhydrous MgSO₄ and concentrated under reduced pressure. The crude material was purified by flash chromatography (SiO₂, 2-10% MeOH/NH₃ in CH₂Cl₂) to afford 2-(7-methoxynaphthalen-1-yl)-N-(3-methylbenzyl)ethan-1-amine as a clear oil (242 mg, 79%). ¹H NMR (400 MHz, CDCl₃) δ 7.76 (d, J=8.9 Hz, 1H), 7.66 (d, J=8.0 Hz, 1H), 7.34-7.31 (m, 1H), 7.31 (d, J=2.5 Hz, 1H), 7.29-7.02 (m, 6H), 3.90 (s, 3H), 3.82 (s, 2H), 3.31-3.23 (m, 2H), 3.09-3.04 (m, 2H), 2.31 (s, 3H).

### Step 3: N-(3-methylbenzyl)-2-(7-methoxynaphthalen-1-yl)ethan-1-amine fumarate (I-19·Fum)

N-(3-methylbenzyl)-2-(7-methoxynaphthalen-1-yl)ethan-1-amine (237 mg, 0.78 mmol) was formulated as the fumarate salt according to general procedure B which provided white crystals (266 mg, 81%). ¹H NMR (400 MHz, DMSO-d₆) δ 7.84 (d, J=9.0 Hz, 1H), 7.76-7.70 (m, 1H), 7.37 (d, J=2.5 Hz, 1H), 7.34 (dd, J=7.1, 1.4 Hz, 1H), 7.30-7.23 (m, 4H), 7.18 (dd, J=8.9, 2.4 Hz, 1H), 7.16-7.11 (m, 1H), 6.54 (s, 2H), 4.02 (s, 2H), 3.88 (s, 3H), 3.39-3.31 (m, 2H), 3.09-3.00 (m, 2H), 2.29 (s, 3H). ¹³C NMR (101 MHz, DMSO-d₆) δ 167.7, 157.6, 137.6, 135.0, 133.2, 132.6, 130.3, 129.9, 128.9, 128.7, 128.4, 127.3, 126.9, 126.3, 123.2, 118.0, 102.4, 55.3, 50.6, 47.4, 30.4, 20.9.

### Example 20: Synthesis of N-Benzyl-2-(7-methoxynaphthalen-1-yl)ethan-1-amine (I-20)

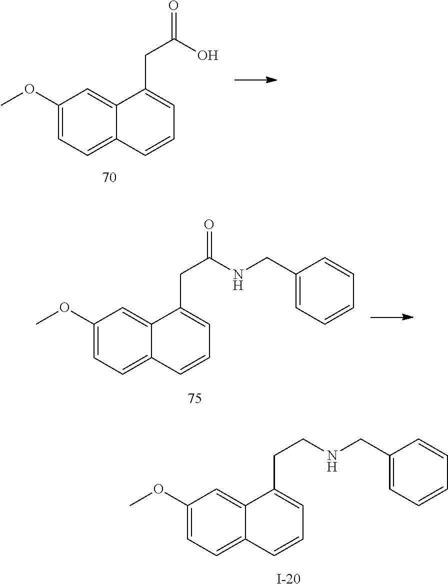

### Step 1: N-Benzyl-2-(7-methoxynaphthalen-1-yl)acetamide (75)

To a solution of (7-methoxy-1-naphthyl)acetic acid (1.0 g, 4.62 mmol) in DMF (5 mL) was added a solution of 2-(3H-[1,2,3]triazolo[4,5-b]pyridin-3-yl)-1,1,3,3-tetramethylisouronium hexafluorophosphate (3.5 g, 9.25 mmol) in DMF (10 mL), followed by iPr₂NEt (3.2 mL, 18.5 mmol) and then benzylamine (0.6 mL, 5.55 mmol). The resulting reaction was stirred at room temperature for 1 h and then diluted with H₂O (100 mL). The precipitate was collected by vacuum filtration and washed with H₂O to afford the title compound (1.4 g, quant.) as an off-white solid. ¹H NMR (400 MHz, DMSO-d₆) δ 8.66 (t, J=5.7 Hz, 1H), 7.84 (d, J=8.9 Hz, 1H), 7.74 (d, J=8.1 Hz, 1H), 7.45-7.36 (m, 2H), 7.32-7.11 (m, 7H), 4.30 (d, J=5.9 Hz, 2H), 3.93 (s, 2H), 3.79 (s, 3H). ¹³C NMR (101 MHz, DMSO-d₆) δ 170.1, 157.31, 139.5, 133.2, 131.5, 123.0, 128.7, 128.5, 128.2, 127.3, 126.8, 126.8, 123.1, 117.8, 103.2, 55.1, 42.3, 40.3.

### Step 2: N-Benzyl-2-(7-methoxynaphthalen-1-yl)ethan-1-amine (I-20)

To a solution of N-benzyl(7-methoxy-1-naphthyl)acetamide (1.4 g, 4.58 mmol) in anhydrous THF (30 mL) under an inert atmosphere was added 2 M borane dimethylsulfide in THF (9.0 mL, 18.3 mmol) and the reaction was stirred at reflux for 1 h. Upon completion, the hot solution was quenched with 6 M aq. HCl dropwise until effervescence ceased, MeOH (5 mL) was then added, followed by an additional portion of HCl (1 mL, 6 M aq. soln). The solution was stirred at reflux for 1 h, and then concentrated under a stream of N₂ gas. The aqueous residue was neutralised with saturated aq. Na₂CO₃ and then made basic with NaOH (1 mL, 15% aq. solution). The suspension was extracted with CH₂Cl₂ (50 mL×3), the combined organic layers were then washed with saturated aq. Na₂CO₃ (20 mL×3), brine (50 mL), and then dried over anhydrous Na₂SO₄, filtered, and the filtrate concentrated in vacuo. The residue was purified by flash chromatography (0.1% to 2% MeOH/NH₃ in CH₂Cl₂) to afford the title compound as a colourless oil (1.23 g, 92%). ¹H NMR (400 MHz, CDCl₃) δ 7.76 (d, J=8.9 Hz, 1H), 7.66 (d, J=8.0 Hz, 1H), 7.37-7.20 (m, 8H), 7.15 (dd, J=8.9, 2.5 Hz, 1H), 3.90 (s, 3H), 3.85 (s, 2H), 3.27 (t, J=7.3 Hz, 2H), 3.07 (t, J=7.3 Hz, 2H). ¹³C NMR (101 MHz, CDCl₃) δ 157.82, 140.31, 134.78, 133.22, 130.42, 129.47, 128.54, 128.21, 127.12, 127.09, 126.82, 123.37, 118.12, 102.60, 55.48, 54.03, 49.51, 33.86.

### Step 3: N-Benzyl-2-(7-methoxynaphthalen-1-yl)ethan-1-amine (I-20·fum)

N-Benzyl-2-(7-methoxynaphthalen-1-yl)ethan-1-amine (200 mg, 0.69 mmol) was formulated as the fumarate salt according to general procedure B which provided the product as white crystals (215 mg, 82%). HPLC Purity: 99.9% (254 nm); ¹H NMR (400 MHz, DMSO-d₆) δ 7.83 (d, J=9.0 Hz, 1H), 7.72 (d, J=7.9 Hz, 1H), 7.45 (dd, J=8.1, 1.3 Hz, 2H), 7.42-7.23 (m, 6H), 7.17 (dd, J=8.9, 2.5 Hz, 1H), 6.53 (s, 1.6H), 4.01 (s, 2H), 3.87 (s, 3H), 3.39-3.26 (m, 2H), 3.09-2.91 (m, 2H). ¹³C NMR (101 MHz, DMSO-d₆) δ 167.9, 157.5, 136.2, 135.1, 133.6, 132.6, 130.2, 129.0, 128.8, 128.4, 127.7, 127.1, 126.7, 123.2, 118.0, 102.4, 55.3, 51.0, 47.7, 30.9.

Scheme 4: Compounds of general formula (I) can be synthesised from an appropriately substituted acenapthylen-1-one following the outlined sequence of steps in Scheme 4 or similar as one skilled in the art may utilise. Baeyer-Villiger oxidation fashions a chromenone intermediate which upon reduction furnishes the appropriately substituted naphthalene. Chemoselective phenolic methylation with methyl iodide followed by partial oxidation of the terminal alcohol by Dess-Martin periodinane generates the required aldehyde intermediate that can subsequently undergo reductive amination to give compounds of general formula (I) (exemplified by Examples I-13, I-22, I-23, I-24, I-25, I-27, I-28, and I-30). Subsequent demethylation the methyl ether allows access to the phenols of general formula (I) (exemplified by example I-21, I-26, I-29 and I-31).

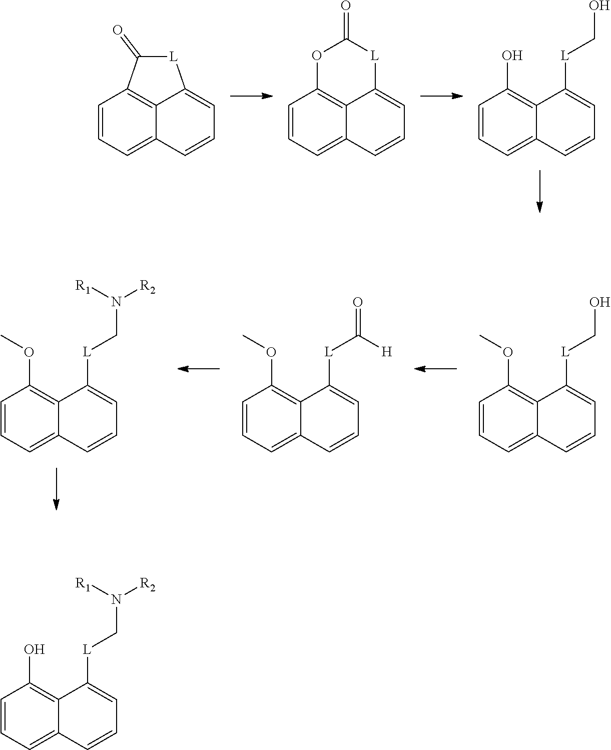

### Example 13: Synthesis of 2-(8-methoxynaphthalen-1-yl)-N,N-dimethylethan-1-amine (I-13)

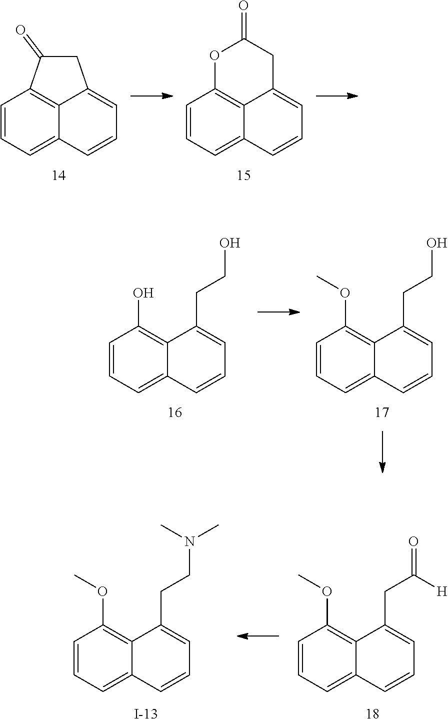

### Step 1: Benzo[de]chromen-2(3H)-one (15)

A solution of acenaphthylen-1(2H)-one (3.5 g, 20.8 mmol) in CH₂Cl₂ (105 mL) was treated with NaHCO₃ (8.74 g, 104 mmol) and 3-chloroperbenzoic acid (80% purity) (8.98 g, 41.6 mmol) and the mixture was refluxed for 18 h. The reaction mixture was diluted with CH₂Cl₂ (50 mL) and washed sequentially with H₂O (150 mL), saturated aq. Na₂S2O₃ solution (130 mL), aq. NaHCO₃ (130 mL), and brine (100 mL). The organic layer was then dried over anhydrous Na₂SO₄, filtered, and concentrated in vacuo. The residue was purified by column chromatography (SiO₂, 1% to 2% EtOAc in petroleum ether) to afford the title compound (2.0 g, 52%) as an off-white solid. ¹H NMR (400 MHz, CDCl₃): δ 7.78 (d, J=8.4 Hz, 1H), 7.62 (d, J=8.0 Hz, 1H), 7.45-7.53 (m, 2H), 7.27 (m, 1H), 7.14 (d, J=7.6 Hz, 1H), 4.31 (s, 2H).

### Step 2: 8-(2-hydroxyethyl)naphthalen-1-ol (16)

A solution of benzo[de]chromen-2(3H)-one (1.4 g, 7.6 mmol) in THF (28 mL) was cooled to 0° C. and treated with LiAlH₄ (288 mg, 7.6 mmol) under N₂. The reaction mixture was stirred at RT for 15 min. Upon completion, the reaction was quenched by addition of Na₂SO₄.10H₂O (1.40 g) and the precipitate was removed by filtration and the filter cake was washed with THF (50 mL). The combined filtrate was concentrated to afford the crude title compound (1.4 g) as a brown solid which was used in the next step without further purification.

### Step 3: 2-(8-methoxynaphthalen-1-yl)ethan-1-ol (17)

A solution of crude 8-(2-hydroxyethyl)naphthalen-1-ol (1.4 g, 7.44 mmol) in DMF (14 mL) was treated with K₂CO₃ (3.08 g, 22.3 mmol) and MeI (2.11 g, 14.9 mmol) at RT and stirred for 12 h. Upon completion, the reaction was quenched by addition of H₂O (50 mL) and extracted with EtOAc (20 mL×2). The combined organic layers were washed with brine (15 mL), dried over anhydrous Na₂SO₄, filtered, and concentrated in vacuo. The residue was purified by column chromatography (SiO₂, 3% to 8% EtOAc in petroleum ether) to afford the title compound (0.7 g, 46% over 2 steps) as an off-white solid. ¹H NMR (400 MHz, CDCl₃): δ 7.70 (d, J=8.4 Hz, 1H), 7.45 (d, J=8.0 Hz, 1H), 7.36-7.40 (m, 2H), 7.27 (m, 1H), 6.87 (d, J=7.2 Hz, 1H), 3.95-3.99 (m, 5H), 3.59 (t, J=6.4 Hz, 2H).

### Step 4: 2-(8-methoxynaphthalen-1-yl)acetaldehyde (18)

A solution of 2-(8-methoxynaphthalen-1-yl)ethan-1-ol (250 mg, 1.24 mmol) in DMF (5 mL) was cooled to 0° C., treated with Dess-Martin periodinane (629 mg, 1.48 mmol) under N₂, and then stirred at RT for 1 h. Upon completion, the reaction was quenched with saturated aq. NaHCO₃ solution (10 mL) and extracted with CH₂Cl₂ (5 mL×3). The combined organic layer was washed with brine (5 mL), dried over anhydrous Na₂SO₄, filtered, and concentrated in vacuo. The residue was purified by preparative TLC (1:1, petroleum ether:EtOAc) to afford the title compound (240 mg, 97%) as a colorless oil. ¹H NMR (400 MHz, CDCl₃): δ 9.82 (s, 1H), 7.78 (d, J=7.6 Hz, 1H), 7.40-7.47 (m, 3H), 7.24 (d, J=6.8 Hz, 1H), 6.85 (d, J=7.6 Hz, 1H), 4.20 (s, 2H), 3.88 (s, 3H).

### Step 5: 2-(8-methoxynaphthalen-1-yl)-N,N-dimethylethan-1-amine (I-13)

A solution of 2-(8-methoxynaphthalen-1-yl)acetaldehyde (240 mg, 1.2 mmol) in CH₂Cl₂ (7 mL) was cooled to 0° C. and treated with 2 M Me₂NH in THF (0.90 mL, 1.8 mmol) under N₂ and stirred for 15 min. NaBH(OAc)₃ (508 mg, 2.4 mmol) was then added to the reaction mixture at 0° C. under N₂ and the reaction was stirred at RT for a further 12 h. Upon completion, the reaction was quenched with MeOH (10 mL) and concentrated in vacuo. The residue was diluted with CH₂Cl₂ (10 mL) and washed with H₂O (4 mL). The organic layer was dried over anhydrous Na₂SO₄, filtered, and concentrated in vacuo. The residue was purified by preparative HPLC (column: Phenomenex C18, 80*40 mm*3 μm; mobile phase: [H₂O (NH₄HCO₃)-ACN]; B %: 10%-40%, 8 min) to afford the title compound (I-13, 59 mg, 21%) as a colourless oil. HPLC purity: 96.6% (220 nm); ¹H NMR (400 MHz, MeOD-d₄) δ 7.66 (d, J=8.0 Hz, 1H), 7.41 (d, J=8.0 Hz, 1H), 7.32-7.37 (m, 2H), 7.25 (d, J=7.2 Hz, 1H), 6.94 (d, J=7.6 Hz, 1H), 3.99 (s, 3H), 3.44-3.49 (m, 2H), 2.59-2.63 (m, 2H), 2.38 (s, 6H); LCMS (ESI+): m/z 230.1 [M+H]⁺.

### Example 21: Synthesis of 8-(2-(dimethylamino)ethyl)naphthalen-1-ol (I-21)

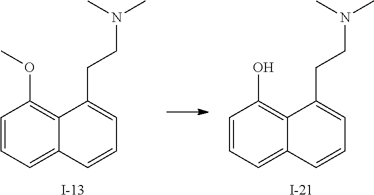

### Step 1: 8-(2-(dimethylamino)ethyl)naphthalen-1-ol (I-21)

A suspension of 2-(8-methoxynaphthalen-1-yl)-N,N-dimethylethan-1-amine (0.4 g, 1.74 mmol) and TBAB (56 mg, 0.17 mmol) in HBr (4 mL) was degassed and purged with N₂ gas before being heated at 100° C. for 2 h. The reaction was then concentrated under reduced pressure and the residue purified by preparative HPLC (column: Waters Xbridge BEH C18, 100*30 mm*10 μm; mobile phase: [H₂O (NH₄HCO₃)-ACN]; B %: 10-50%, over 8 min) to give the title compound as a brown solid (I-21, 20 mg, 7%). HPLC purity: 98.0% (220 nm); ¹H NMR 5 (400 MHz, MeOD-d₄) 7.61 (d, J=8.2 Hz, 1H), 7.17-7.32 (m, 4H), 6.78-6.84 (m, 1H), 3.50-3.56 (m, 2H), 2.71-2.79 (m, 2H), 2.38 (s, 6H). LCMS (ESI+): m/z 216.1 [M+H]⁺ (1.83 min).

### Example 22: Synthesis of N,N-diethyl-2-(8-methoxynaphthalen-1-yl)ethan-1-amine (I-22)

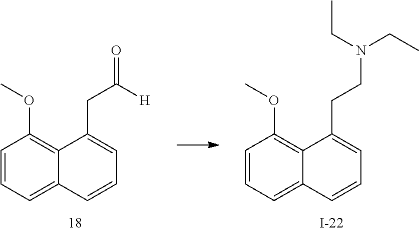

### Step 1: N,N-Diethyl-2-(8-methoxynaphthalen-1-yl)ethan-1-amine (I-22)

To a solution of 2-(8-methoxynaphthalen-1-yl)acetaldehyde (0.30 g, 1.50 mmol) in CH₂Cl₂ (9.0 mL) was added Et₂NH (165 mg, 2.26 mmol) at 0° C. under N₂. The reaction mixture was stirred at RT for 15 min. Then, NaBH(OAc)₃ (636 mg, 3.0 mmol) was added at 0° C. under N₂ and the reaction mixture was stirred at RT for 12 h. The reaction mixture was quenched with MeOH (20 mL) and the volatiles were removed under reduced pressure. The residue was dissolved in CH₂Cl₂ (30 mL), washed with H₂O (10 mL) before being dried over anhydrous Na₂SO₄, filtered and concentrated to obtain the crude product. The crude product was purified by preparative HPLC (column: Waters Xbridge OBD C18, 150*40 mm*10 μm; mobile phase: [H₂O(NH₄HCO₃)-ACN]; B %: 25-55%, 8 min) to obtain the title compound (I-22, 222 mg, 58%) as a brown oil. HPLC purity: 98.6% (220 nm); ¹H NMR (400 MHz, MeOD-d₄) δ 7.66 (d, J=8.0 Hz, 1H), 7.41 (d, J=8.0 Hz, 1H), 7.37-7.32 (m, 2H), 7.25 (d, J=7.2 Hz, 1H), 6.94 (d, J=7.6 Hz, 1H), 3.98 (s, 3H), 3.47-3.43 (m, 2H), 2.77-2.68 (m, 6H), 1.14 (t, J=7.2 Hz, 6H). LCMS (ESI+): m/z 258.0 [M+H]⁺.

### Example 23: Synthesis of N-(2-(8-methoxynaphthalen-1-yl)ethyl)-N-propylpropan-1-amine (I-23)

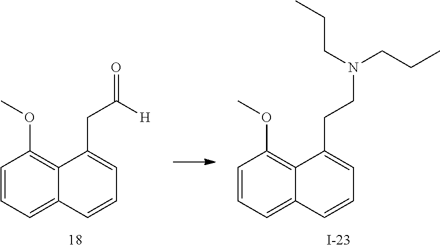

### Step 1: N-(2-(8-methoxynaphthalen-1-yl)ethyl)-N-propylpropan-1-amine (I-23)

To a solution of 2-(8-methoxynaphthalen-1-yl)acetaldehyde (0.30 g, 1.50 mmol) in CH₂Cl₂ (9.0 mL) was added Pr₂NH (228 mg, 2.25 mmol) at 0° C. under N₂. The reaction mixture was stirred at RT for 15 min and then cooled to 0° C. NaBH(OAc)₃ (636 mg, 3.0 mmol) was then added and the resulting suspension was stirred at RT for 12 h. The reaction mixture was then quenched with MeOH (20 mL) and the volatiles were removed in vacuo. The residue was dissolved in CH₂Cl₂ (20 mL), washed with H₂O (5 mL) and the organic layer dried over anhydrous Na₂SO₄, filtered and concentrated to obtain the crude product. The crude product was purified by preparative HPLC (column: Waters Xbridge OBD C18, 150 mm*40 mm*10 μm; mobile phase: [H₂O (NH₄HCO₃)-ACN]; B %: 40-70%, over 8 min) to obtain the title compound (I-23, 190 mg, 44%) as a brown oil. HPLC purity: 98.5% (220 nm); ¹H NMR (400 MHz, MeOD-d₄) δ 7.65 (d, J=8.4 Hz, 1H), 7.41 (d, J=8.0 Hz, 1H), 7.37-7.32 (m, 2H), 7.24 (d, J=7.2 Hz, 1H), 6.94 (d, J=7.6 Hz, 1H), 3.98 (s, 3H), 3.47-3.43 (m, 2H), 2.78-2.74 (m, 2H), 2.59-2.55 (m, 4H), 1.60-1.54 (m, 4H), 0.95 (t, J=7.4 Hz, 6H). LCMS (ESI+): m/z 286.1 [M+H]⁺.

### Example 24: Synthesis of N-isopropyl-N-(2-(8-methoxynaphthalen-1-yl)ethyl)propan-2-amine (I-24)

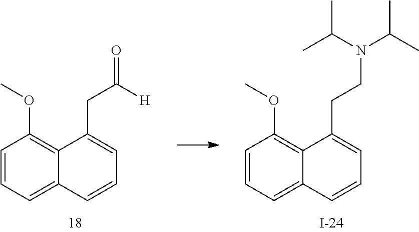

### Step 1: N-isopropyl-N-(2-(8-methoxynaphthalen-1-yl)ethyl)propan-2-amine (I-24)

To a solution of 2-(8-methoxynaphthalen-1-yl)acetaldehyde (0.30 g, 1.50 mmol) in CH₂Cl₂ (9.0 mL) was added iPr₂NH (228 mg, 2.25 mmol) at 0° C. under N₂. The reaction mixture was stirred at RT for 15 min and then cooled to 0° C. NaBH(OAc)₃ (636 mg, 3.0 mmol) was then added and the resulting suspension was stirred at RT for 12 h. The reaction mixture was then quenched with MeOH (20 mL) and the volatiles were removed in vacuo. The residue was dissolved in CH₂Cl₂ (30 mL), washed with H₂O (10 mL) and the organic layer dried over anhydrous Na₂SO₄, filtered and concentrated to obtain the crude product. The crude product was purified by preparative HPLC (column: Waters Xbridge OBD C18, 150 mm*40 mm*10 μm; mobile phase: [H₂O (NH₄HCO₃)-ACN]; B %: 30-60%, over 8 min) to obtain the title compound (I-24, 182 mg, 42%) as a brown oil. HPLC purity: 97.8% (220 nm); ¹H NMR (400 MHz, MeOD-d₄) δ 7.66 (d, J=8.4 Hz, 1H), 7.41 (d, J=8.0 Hz, 1H), 7.37-7.32 (m, 2H), 7.24 (d, J=7.2 Hz, 1H), 6.94 (d, J=7.6 Hz, 1H), 3.98 (s, 3H), 3.45-3.41 (m, 2H), 3.21-3.15 (m, 2H), 2.75-2.71 (m, 2H), 1.11 (d, J=6.8 Hz, 12H). LCMS (ESI+): m/z 286.1 [M+H]⁺, 2.18 min.

### Example 25: Synthesis of N-ethyl-2-(8-methoxynaphthalen-1-yl)-N-methylethan-1-amine (I-25)

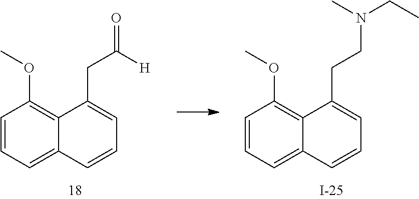

### Step 1: N-ethyl-2-(8-methoxynaphthalen-1-yl)-N-methylethan-1-amine (I-25)

To a solution of 2-(8-methoxynaphthalen-1-yl)acetaldehyde (0.30 g, 1.50 mmol) in CH₂Cl₂ (9 mL) was added EtNHMe (133 mg, 2.25 mmol) at 0° C. under N₂. The reaction mixture was stirred at RT for 15 min and then cooled to 0° C. NaBH(OAc)₃ (636 mg, 3 mmol) was then added and the resulting suspension was allowed to stir at RT for 12 h. The reaction mixture was then quenched with MeOH (20 mL) and the volatiles were removed in vacuo. The residue was dissolved in CH₂Cl₂ (30 mL), washed with H₂O (10 mL) and the organic layer dried over anhydrous Na₂SO₄, filtered and concentrated to obtain the crude product which was purified by preparative HPLC (column: Waters Xbridge OBD C18, 150 mm*40 mm*10 μm; mobile phase: [H₂O (NH₄HCO₃)-ACN]; B %: 25-55%, over 8 min) to obtain the title compound (I-25, 124 mg, 33%) as a brown oil. HPLC purity: 96.7% (220 nm); ¹H NMR (400 MHz, MeOD-d₄) δ 7.66 (d, J=8.4 Hz, 1H), 7.41 (d, J=8.0 Hz, 1H), 7.37-7.32 (m, 2H), 7.24 (d, J=7.2 Hz, 1H), 6.94 (d, J=7.6 Hz, 1H), 3.99 (s, 3H), 3.49-3.45 (m, 2H), 2.70-2.65 (m, 2H), 2.60-2.56 (m, 2H), 2.41 (s, 3H), 1.14 (t, J=7.2 Hz, 3H). LCMS (ESI+): m/z 244.2 [M+H]⁺, 1.42 min.

### Example 26: Synthesis of 8-(2-(methyl(ethyl)amino)ethyl)naphthalen-1-ol (I-26)

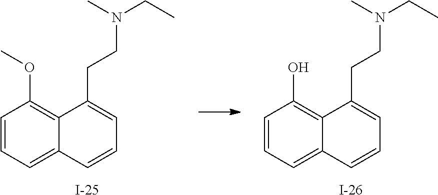

### Step 1: 8-(2-(methyl(ethyl)amino)ethyl)naphthalen-1-ol (I-26)

A suspension of N-ethyl-2-(8-methoxynaphthalen-1-yl)-N-methylethan-1-amine (0.4 g, 1.64 mmol) and TBAB (52 mg, 0.16 mmol) in HBr (4 mL) was degassed and purged with N₂ gas before being heated at 100° C. for 2 h. The reaction was then concentrated under reduced pressure and the residue purified by preparative HPLC (column: Waters Xbridge BEH C18, 250*50 mm*10 μm; mobile phase: [H₂O (NH₄HCO₃)-ACN]; B %: 25-55%, over 10 min) to give the title compound as a brown solid (I-26, 20 mg, 5%). HPLC purity: 98.0% (220 nm); ¹H NMR (400 MHz, MeOD-d₄) δ 7.62 (d, J=8.2 Hz, 1H), 7.27-7.32 (m, 2H), 7.17-7.25 (m, 2H), 6.79-6.83 (m, 1H), 3.49-3.56 (m, 2H), 2.80-2.87 (m, 2H), 2.57-2.63 (m, 2H), 2.39 (s, 3H), 1.11 (t, J=7.2 Hz, 3H); LCMS (ESI+): m/z 230.1 [M+H]⁺, 1.87 min.

### Example 27: Synthesis of N-(2-(8-methoxynaphthalen-1-yl)ethyl)-N-methylpropan-1-amine (I-27)

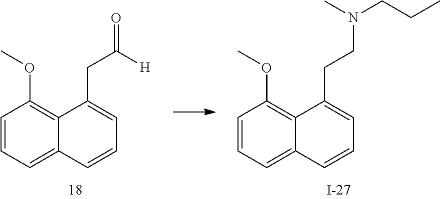

### Step 1: N-(2-(8-methoxynaphthalen-1-yl)ethyl)-N-methylpropan-1-amine (I-27)

To a solution of 2-(8-methoxynaphthalen-1-yl)acetaldehyde (0.30 g, 1.50 mmol) in CH₂Cl₂ (9.0 mL) was added N-methylpropan-1-amine (165 mg, 2.26 mmol) at 0° C. under N₂. The reaction mixture was then stirred at RT for 15 min and then cooled to 0° C. NaBH(OAc)₃ (636 mg, 3.0 mmol) was then added and the resulting suspension was stirred at RT for 12 h. The reaction mixture was then quenched with MeOH (20 mL) and the volatiles were removed in vacuo. The residue was dissolved in CH₂Cl₂ (20 mL), washed with H₂O (5 mL) and the organic layer dried over anhydrous Na₂SO₄, filtered and concentrated to obtain the crude product which was purified by preparative HPLC (column: Waters Xbridge OBD C18, 100 mm*30 mm*10 μm; mobile phase: [H₂O (NH₄HCO₃)-ACN]; B %: 30-70%, over 8 min) to obtain the title compound (I-27, 149 mg, 37%) as a brown oil. HPLC purity: 96.6% (220 nm); ¹H NMR (400 MHz, MeOD-d₄) δ 7.68 (d, J=8.4 Hz, 1H), 7.42 (d, J=8.0 Hz, 1H), 7.39-7.34 (m, 2H), 7.28 (d, J=7.2 Hz, 1H), 6.97 (d, J=7.6 Hz, 1H), 4.01 (s, 3H), 3.51-3.47 (m, 2H), 2.73-2.68 (m, 2H), 2.52-2.48 (m, 2H), 2.44 (s, 3H), 1.65-1.58 (m, 2H), 0.97 (t, J=7.4 Hz, 3H). LCMS (ESI+): m/z 258.0 [M+H]⁺, 1.6 min.

### Example 28: Synthesis of N-(2-(8-methoxynaphthalen-1-yl)ethyl)-N-methylpropan-2-amine (I-28)

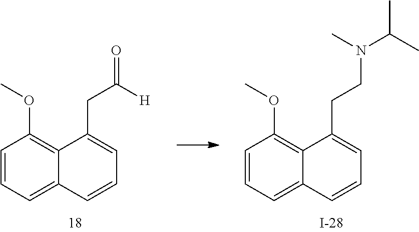

### Step 1: N-(2-(8-methoxynaphthalen-1-yl)ethyl)-N-methylpropan-2-amine (I-28)

To a solution of 2-(8-methoxynaphthalen-1-yl)acetaldehyde (0.30 g, 1.50 mmol) in CH₂Cl₂ (9.0 mL) was added iPrNHMe (165 mg, 2.26 mmol) and the reaction mixture was stirred at RT for 15 min before being cooled to 0° C. NaBH(OAc)₃ (636 mg, 3.0 mmol) was then added and the resulting suspension was stirred at RT for 12 h. The reaction mixture was then quenched with MeOH (20 mL) and the volatiles were removed in vacuo. The residue was dissolved in CH₂Cl₂ (30 mL), washed with H₂O (10 mL) and the organic layer dried over anhydrous Na₂SO₄, filtered and concentrated to obtain the crude product which was purified by preparative HPLC (column: Waters Xbridge OBD C18, 150 mm*40 mm*10 μm; mobile phase: [H₂O (NH₄HCO₃)-ACN]; B %: 25-55%, over 8 min) to obtain the title compound (I-28, 216 mg, 56%) as a brown oil. HPLC purity: 100% (220 nm); ¹H NMR (400 MHz, MeOD-d₄) δ 7.66 (d, J=8.4 Hz, 1H), 7.38 (d, J=8.0 Hz, 1H), 7.35-7.32 (m, 2H), 7.24 (d, J=7.2 Hz, 1H), 6.94 (d, J=7.6 Hz, 1H), 3.98 (s, 3H), 3.47-3.43 (m, 2H), 2.95-2.92 (m, 1H), 2.69-2.65 (m, 2H), 2.40 (s, 3H), 1.08 (t, J=6.4 Hz, 6H). LCMS (ESI+): m/z: 258.1 [M+H]⁺, 1.4 min.

### Example 29: Synthesis of 8-(2-(methyl(isopropyl)amino)ethyl)naphthalen-1-ol (I-29)

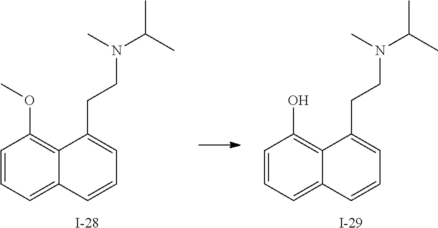

### Step 1: 8-(2-(methyl(isopropyl)amino)ethyl)naphthalen-1-ol (I-29·HCl)

A suspension of N-(2-(8-methoxynaphthalen-1-yl)ethyl)-N-methylpropan-2-amine (0.4 g, 1.55 mmol) and TBAB (50 mg, 0.16 mmol) in HBr (4 mL) was degassed and purged with N₂ gas before being heated at 100° C. for 2 h. The reaction was then concentrated under reduced pressure and major impurities were first removed from the obtained residue by preparative HPLC (column: Waters Xbridge BEH C18, 100*30 mm*10 μm; mobile phase: [H₂O (NH₄HCO₃)-ACN]; B %: 10-40%, over 8 min). The obtained crude product was further purified by preparative HPLC (column: Waters Xbridge BEH C18, 100*30 mm*10 μm; mobile phase: [H₂O (HCl)-ACN]; B %: 10-40%, over 8 min) to give the title compound as the hydrochloride salt which was an off-white solid (I-29, 20 mg, 5%). HPLC purity: 100% (220 nm); ¹H NMR (400 MHz, MeOD-d₄) δ 7.72 (d, J=8.2 Hz, 1H), 7.26-7.38 (m, 4H), 6.90 (d, J=7.4 Hz, 1H), 3.65-3.80 (m, 3H), 3.52-3.60 (m, 1H), 3.23-3.29 (m, 1H), 2.91 (s, 3H), 1.37 (t, J=6.4 Hz, 6H); LCMS (ESI+): m/z 244.2 [M+H]⁺, 1.94 min.

### Example 30: Synthesis of N-(2-(8-methoxynaphthalen-1-yl)ethyl)-N-methylcyclopropanamine (I-30)

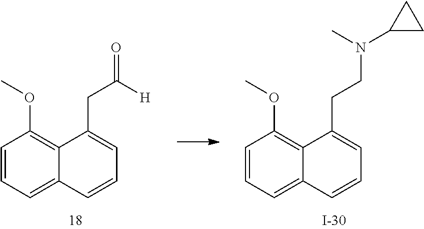

### Step 1: N-(2-(8-methoxynaphthalen-1-yl)ethyl)-N-methylcyclopropanamine (I-30)

A mixture of 2-(8-methoxynaphthalen-1-yl)acetaldehyde (0.30 g, 1.50 mmol), NaBH(OAc)₃ (0.63 g, 3.00 mmol) and N-methylcyclopropanamine (159 mg, 2.25 mmol) in CH₂Cl₂ (9 mL) was degassed and stirred at 25° C. for 12 h under a N₂ atmosphere. The mixture was diluted with H₂O (50 mL) and extracted with petroleum ether (30 mL×3). The combined organic extracts were washed with H₂O (50 mL×3), dried over Na₂SO₄, filtered and concentrated under reduced pressure to give a residue which was purified by preparative HPLC (column: Waters Xbridge Prep OBD C18, 150*40 mm*10 μm; mobile phase: [H₂O (NH₄HCO₃)-ACN]; B %: 40-70%, over 8.0 min) to provide N-(2-(8-methoxynaphthalen-1-yl)ethyl)-N-methylcyclopropanamine (0.30 g, 80% yield) as a brown oil. HPLC purity: 97.8% (220 nm); ¹H NMR (400 MHz, MeOD-d₄) δ7.66 (d, J=8.2 Hz, 1H), 7.40-7.44 (m, 1H), 7.31-7.38 (m, 2H), 7.24 (d, J=7.0 Hz, 1H), 6.95 (d, J=7.4 Hz, 1H), 3.99 (s, 3H), 3.49-3.54 (m, 2H), 2.79-2.85 (m, 2H), 2.51 (s, 3H), 1.81-1.88 (m, 1H), 0.49-0.60 (m, 4H). LCMS (ESI+): m/z 256.2 [M+H]⁺.

### Example 31: Synthesis of 8-(2-(cyclopropyl(methyl)amino)ethyl)naphthalen-1-ol (I-31)

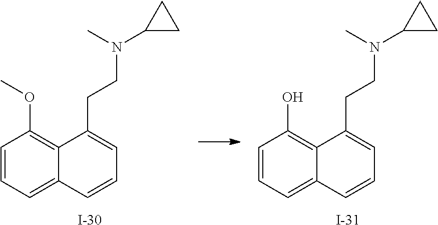

### Step 1: 8-(2-(cyclopropyl(methyl)amino)ethyl)naphthalen-1-ol (I-31)

A mixture of N-(2-(8-methoxynaphthalen-1-yl)ethyl)-N-methylcyclopropanamine (0.30 g, 1.17 mmol) and TBAB (0.04 mg, 0.12 μmol) in HBr (3.0 mL) was degassed and purged with N₂, before being stirred at 100° C. for 2 h under an atmosphere of N₂. The reaction was then concentrated under reduced pressure and the resultant residue purified by preparative HPLC (column: Waters Xbridge BEH C18, 100*30 mm*10 μm; mobile phase: [H₂O (NH₄HCO₃)-ACN]; B %: 25-55%, over 8.0 min) to provide 8-(2-(cyclopropyl(methyl)amino)ethyl)naphthalen-1-ol (0.02 g, 7%) as a brown solid. HPLC purity: 95.8% (220 nm); ¹H NMR (400 MHz, MeOD-d₄) δ 7.61 (d, J=8.2 Hz, 1H), 7.27-7.32 (m, 2H), 7.16-7.24 (m, 2H), 6.81 (d, J=7.4 Hz, 1H), 3.52-3.57 (m, 2H), 2.90-2.96 (m, 2H), 2.48 (s, 3H), 1.83-1.91 (m, 1H), 0.46-0.56 (m, 4H). LCMS (ESI+): m/z 242.1 [M+H]⁺.

Scheme 5: Compounds of general formula (I) wherein Z¹ or Z⁴ is N can be synthesised from the appropriately substituted quinoline following the general sequence of steps shown in Scheme 5 or similar as one skilled in the art may utilise. Variability in the R⁹ position can be achieved from either commercially available stock, or from nucleophilic aromatic substitution conditions, such as with the specific examples outlined below. Similarly, accessing the aryl bromide can be accomplished in various ways known to the skilled person and specific examples are outlined below. Alternatively, the aryl bromide may be available commercially. Subjecting the arylbromide intermediates to Heck coupling coupling conditions followed by alkene reduction provides phthalimide protected intermediates which upon removal allows access to amine analogues that can be alkylated in several ways. One such example includes reductive alkylation with formaldehyde to generate compounds of general formula (I) (exemplified by Example I-8). These steps can also be applied to other appropriately substituted 6,6-aromatic systems.

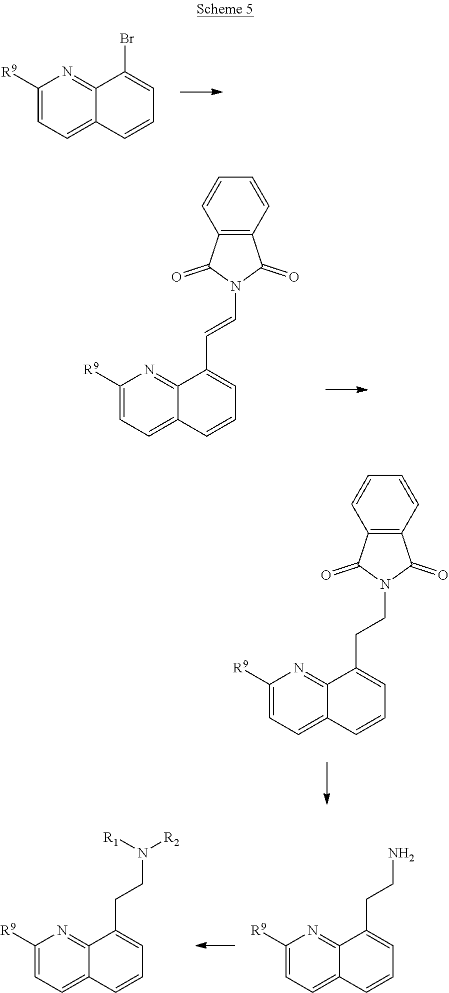

### Example 8: Synthesis of 2-(2-methoxyquinolin-8-yl)-N,N-dimethylethan-1-amine (I-8)

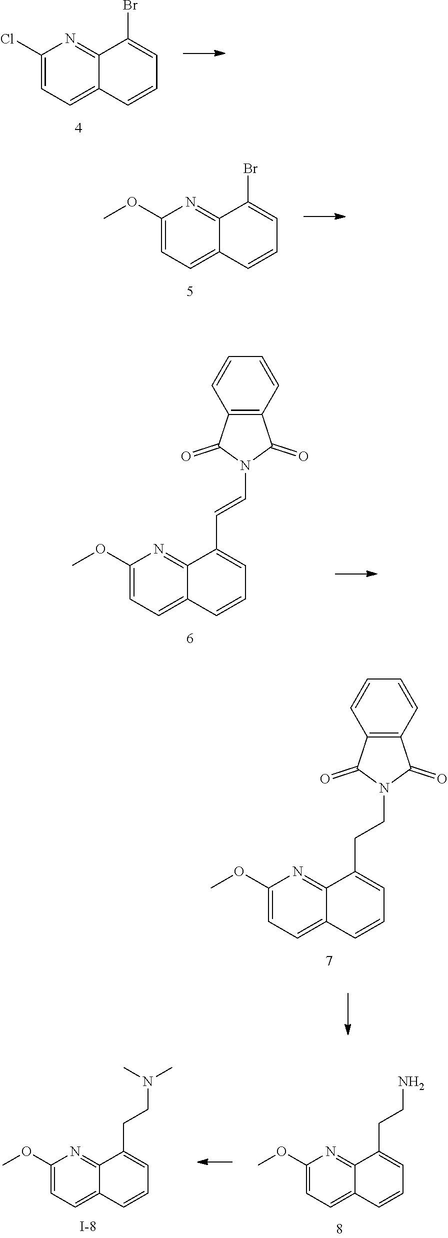

### Step 1: 8-bromo-2-methoxyquinoline (5)

A solution of 8-bromo-2-chloroquinoline (5.0 g, 20.6 mmol) in methanol (40 mL) was treated with NaOMe (6.68 g, 124 mmol) and stirred at 65° C. for 12 h. The reaction mixture was concentrated in vacuo and the residue was diluted with EtOAc (50 mL) and H₂O (50 mL). The layers were separated, and the organic layer washed with brine (30 mL), dried over anhydrous Na₂SO₄, filtered, and concentrated in vacuo to afford 8-bromo-2-methoxyquinoline (4.6 g, 94%) as white solid. ¹H NMR (400 MHz, CDCl₃) δ 7.93-8.00 (m, 2H), 7.66-7.72 (m, 1H), 7.24 (t, J=8.0 Hz, 1H), 6.96 (d, J=8.8 Hz, 1H), 4.16 (s, 3H).

### Step 2: (E)-2-(2-(2-methoxyquinolin-8-yl)vinyl)isoindoline-1,3-dione (6)

A mixture of 8-bromo-2-methoxyquinoline (2.0 g, 8.4 mmol), N-vinylphthalimide (2.18 g, 12.6 mmol), Et₃N (3.4 g, 33.6 mmol), tri-o-tolylphosphine (256 mg, 0.84 mmol) and Pd(OAc)₂ (189 mg, 0.84 mmol) in DMF (15 mL) was degassed and purged with N₂ then stirred at 110° C. for 14 h. Upon completion, the reaction mixture was diluted with ACN (50 mL), and the insoluble material was collected by filtration and the filtrate was discarded. The collected solid was triturated with THF (20 mL) and the remaining insoluble material removed by filtration. The filtrate was concentrated in vacuo and the residue triturated with CH₂Cl₂ (5 mL) to afford (E)-2-(2-(2-methoxyquinolin-8-yl)vinyl)isoindoline-1,3-dione (960 mg, 35%) as a yellow solid. ¹H NMR (400 MHz, CDCl₃) δ 8.50 (d, J=15.2 Hz, 1H), 8.27 (d, J=15.2 Hz, 1H), 8.01 (d, J=8.8 Hz, 1H), 7.93 (dd, J=5.6, 3.2 Hz, 2H), 7.82 (d, J=7.2 Hz, 1H), 7.78 (dd, J=5.6, 3.2 Hz, 2H), 7.65 (d, J=7.6 Hz, 1H), 7.36-7.42 (m, 1H), 6.97 (d, J=8.8 Hz, 1H), 4.23 (s, 3H).

### Step 3: 2-(2-(2-methoxyquinolin-8-yl)ethyl)isoindoline-1,3-dione (7)

10% Pd/C (0.2 g, 0.18 mmol) was added to a solution of (E)-2-(2-(2-methoxyquinolin-8-yl)vinyl)isoindoline-1,3-dione (960 mg, 2.91 mmol) in THF (20 mL) under Ar atmosphere. The suspension was degassed and purged with H₂ three times, and then stirred at RT under H₂ (25 psi) for 12 h. Upon completion, the reaction mixture was filtered, and the filtrate was concentrated in vacuo to afford 2-(2-(2-methoxyquinolin-8-yl)ethyl)isoindoline-1,3-dione (0.9 g) as a light yellow solid and was used in the next step without further purification.

### Step 4: 2-(2-methoxyquinolin-8-yl)ethan-1-amine (8)

A solution of 2-(2-(2-methoxyquinolin-8-yl)ethyl)isoindoline-1,3-dione (0.8 g) in EtOH (8.0 mL) was treated with hydrazine hydrate (1.51 g, 80% purity, 24.1 mmol) at RT. The reaction mixture was then stirred at 80° C. for 12 h. Upon completion, the reaction was filtered, and filtrate was concentrated in vacuo. The residue was diluted with EtOAc (5 mL) and washed with H₂O (5 mL×2). The organic layer was dried over anhydrous Na₂SO₄, filtered, and concentrated in vacuo to afford 2-(2-methoxyquinolin-8-yl)ethan-1-amine (0.4 g, 68% over 2 steps) as a light yellow oil. ¹H NMR (400 MHz, CDCl₃) δ 7.98 (d, J=8.8 Hz, 1H), 7.61 (dd, J=8.0, 1.2 Hz, 1H), 7.50 (d, J=7.2 Hz, 1H), 7.29-7.35 (m, 1H), 6.91 (d, J=8.8 Hz, 1H), 4.07 (s, 3H), 3.30-3.35 (m, 2H), 3.25-3.20 (m, 2H).

### Step 5: 2-(2-methoxyquinolin-8-yl)-N,N-dimethylethan-1-amine (I-8)

A solution of 2-(2-methoxyquinolin-8-yl)ethan-1-amine (0.3 g, 1.48 mmol) in CH₂Cl₂ (5 mL) was treated with AcOH (178 mg, 2.97 mmol), NaBH(OAc)₃ (629 mg, 2.97 mmol), and aq. formaldehyde (37% w/w, 301 mg, 3.71 mmol) at 0° C. and then stirred at RT for 2 h at which point the reaction mixture was concentrated in vacuo and the residue was purified by preparative HPLC (column: Phenomenex Luna C18, 75*30 mm*3 μm; mobile phase: [H₂O (NH₄HCO₃)-ACN]; B %: 10%-45%, 8 min) to afford 2-(2-methoxyquinolin-8-yl)-N,N-dimethylethan-1-amine (I-8, 52 mg, 15%) as a yellow oil. HPLC purity: 99.5% (220 nm); LCMS (ESI+): m/z 231.1 [M+H]⁺; ¹H NMR (400 MHz, CDCl₃) δ 7.97 (d, J=8.8 Hz, 1H), 7.59 (dd, J=8.0, 1.2 Hz, 1H), 7.52 (dd, J=7.2, 1.2 Hz, 1H), 7.31 (m, 1H), 6.90 (d, J=8.8 Hz, 1H). 4.08 (s, 3H), 3.33-3.43 (m, 2H), 2.69-2.78 (m, 2H), 2.40 (s, 6H).

Scheme 6: Compounds of general formula (I) wherein Z⁴ is N can be synthesised from the appropriately substituted quinoline following the outlined sequence of steps in Scheme 6 or similar as one skilled in the art may utilise. Bromination of quinoline derivatives followed by a Heck coupling generates intermediate alkene compounds which upon reductive conditions generates the phthalimide protected amines. Phthalimide removal under standard conditions provides amine derivatives that can be alkylated in several ways. One such example includes reductive alkylation with formaldehyde to generate compounds of general formula (I) (exemplified by Example I-9). These steps can also be applied to other appropriately substituted 6,6-aromatic systems.

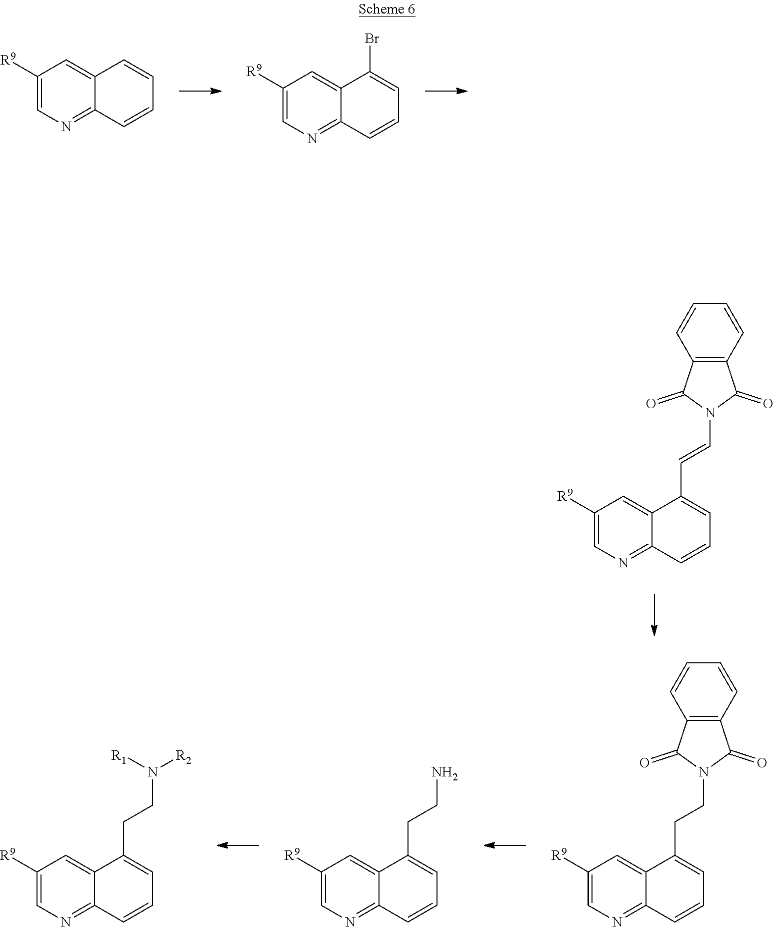

### Example 9: Synthesis of 2-(3-methoxyquinolin-5-yl)-N,N-dimethylethan-1-amine (I-9)

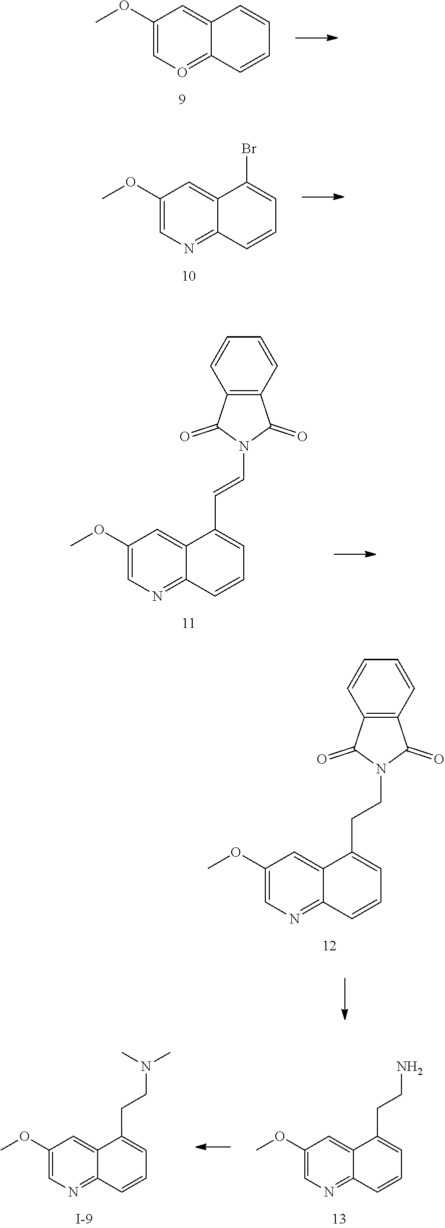

### Step 1: 5-bromo-3-methoxyquinoline (10)

A solution of 3-methoxyquinoline (3.0 g, 18.8 mmol) in concentrated H₂SO₄ (12 mL) was treated with NBS (3.35 g, 18.8 mmol) at 0° C. and then stirred at RT for 4 h. Upon completion, the reaction mixture was cooled to 0° C. and the pH was adjusted to 7 with 3 M aq. NaOH. The aqueous mixture was then extracted with EtOAc (150 mL×2) and the combined organic layer was dried over anhydrous Na₂SO₄, filtered, and concentrated in vacuo. The residue was purified by reverse-phase preparative HPLC to afford the title compound (2.8 g, 62%) as a white solid. ¹H NMR (400 MHz, CDCl₃) δ8.68 (d, J=2.8 Hz, 1H), 8.03 (d, J=8.4 Hz, 1H), 7.81 (d, J=7.6 Hz, 1H), 7.72 (d, J=2.4 Hz, 1H), 7.42 (m, 1H), 4.01 (s, 3H).

### Step 2: (E)-2-(2-(3-methoxyquinolin-5-yl)vinyl)isoindoline-1,3-dione (11)

A mixture of 5-bromo-3-methoxyquinoline (1.0 g, 4.2 mmol), N-vinylphthalimide (1.09 g, 6.3 mmol), Et₃N (1.7 g, 16.8 mmol), tri-o-tolylphosphine (128 mg, 0.42 mmol), Pd(OAc)₂ (94 mg, 0.42 mmol) in DMF (7 mL) was degassed and purged with N₂ three times, and then stirred at 115° C. for 16 h, under N₂. Upon completion, the reaction mixture was diluted with ACN (50 mL), and the insoluble material was collected by filtration and the filtrate was discarded. The collected solid was washed with THF (20 mL) and the remaining insoluble material removed by filtration. The filtrate was concentrated in vacuo and the residue triturated with CH₂Cl₂ (3.0 mL) to afford the title compound (400 mg) as a yellow solid which was used in the next step without further purification.

### Step 3: 2-(2-(3-methoxyquinolin-5-yl)ethyl)isoindoline-1,3-dione (12)

10% Pd/C (0.1 g, 0.09 mmol) was added to a solution of (E)-2-(2-(3-methoxyquinolin-5-yl)vinyl)isoindoline-1,3-dione (0.5 g, 1.51 mmol) in THF (10 mL) under Ar. The suspension was degassed and purged with H₂ three times, and then stirred at RT under H₂ (25 psi) for 12 h. Upon completion, the reaction mixture was filtered and the filtrate was concentrated in vacuo to afford the title compound as a light yellow solid (360 mg) and was used in the next step without further purification.

### Step 4: 2-(3-methoxyquinolin-5-yl)ethan-1-amine (13)

A solution of 2-(2-(3-methoxyquinolin-5-yl)ethyl)isoindoline-1,3-dione (260 mg, 0.78 mmol) in EtOH (5.0 mL) was treated with hydrazine hydrate (490 mg, 7.82 mmol) at RT. The reaction mixture was then stirred at 80° C. for 12 h. Upon completion, the reaction was filtered, and filtrate was concentrated in vacuo. The residue was diluted with EtOAc (5.0 mL) and washed with H₂O (5.0 mL×2) before being dried over anhydrous Na₂SO₄, filtered, and concentrated in vacuo to afford the title compound (110 mg, 13% over 3 steps) as a light-yellow oil. ¹H NMR (400 MHz, CDCl₃) δ 8.70 (d, J=2.8 Hz, 1H), 7.96 (d, J=8.4 Hz, 1H), 7.58 (d, J=2.8 Hz, 1H), 7.47-7.53 (m, 1H), 7.37-7.42 (m, 1H), 3.98 (s, 3H), 3.19 (m, 4H).

### Step 5: 2-(3-methoxyquinolin-5-yl)-N,N-dimethylethan-1-amine (I-9)

A solution of 2-(3-methoxyquinolin-5-yl)ethan-1-amine (110 mg, 0.54 mmol) in CH₂Cl₂ (3.0 mL) was treated with acetic acid (65 mg, 1.09 mmol), NaBH(OAc)₃ (231 mg, 1.09 mmol), and aq. formaldehyde (37% w/w, 110 mg, 1.36 mmol) at 0° C. and then stirred at RT for 2 h. Upon completion, the reaction mixture was concentrated in vacuo and the residue was purified by preparative HPLC (column: Phenomenex Luna C18, 75*30 mm*3 μm; mobile phase: [H₂O (NH₄HCO₃)-ACN]; B %: 10%-40%, 8 min) to afford the title compound (I-9, 26 mg, 21%) as a yellow oil. HPLC purity: 96.0% (220 nm); LCMS (ESI+): m/z 231.1 [M+H]⁺; ¹H NMR (400 MHz, CDCl₃) δ 8.69 (d, J=2.8 Hz, 1H), 7.94 (d, J=8.4 Hz, 1H), 7.59 (d, J=2.4 Hz, 1H), 7.48 (m, 1H), 7.39 (d, J=7.2 Hz, 1H), 3.98 (s, 3H), 3.18-3.26 (m, 2H), 2.62-2.69 (m, 2H), 2.39 (s, 6H).

Scheme 7: Compounds of general formula (I) can be synthesised from an appropriately substituted cyclic ketone following the outlined sequence of steps in Scheme 7 or similar as one skilled in the art may utilise. A 3,4-dihydronaphthalen-1(2H)-one with the appropriate aromatic substitution pattern can be reacted with the phosphonate carbanion of triethyl phosphonate, generated in situ with a strong base, which yields the corresponding substituted cyclohexene after elimination. The substituted cyclohexene can be oxidised with 2,3-dichloro-5,6-dicyano-p-benzoquinone (DDQ) to the corresponding 6,6-aromatic system which can undergo hydrolysis to give the carboxylic acid. The carboxylic acid can then be reduced to the alcohol which is then converted to an alkyl bromide for alkylation with the appropriately substituted amine to give compounds of general formula (I) (exemplified by Example I-32). Alternatively, the carboxylic acid can be activated and reacted directly with the appropriately substituted amine to afford an amide which can be reduced to the desired tertiary amine, which are compounds of general formula (I) (exemplified by Examples I-33, I-34, I-35, I-37, and I-39).

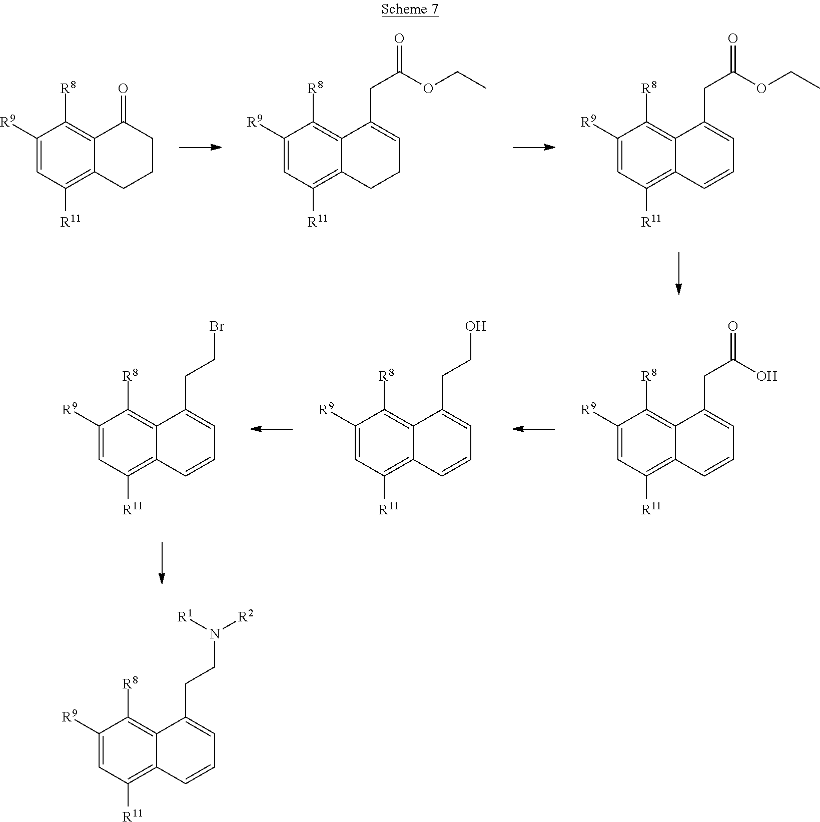

### Example 32: Synthesis of 2-(7-chloronaphthalen-1-yl)-N,N-dimethylethan-1-amine (I-32)

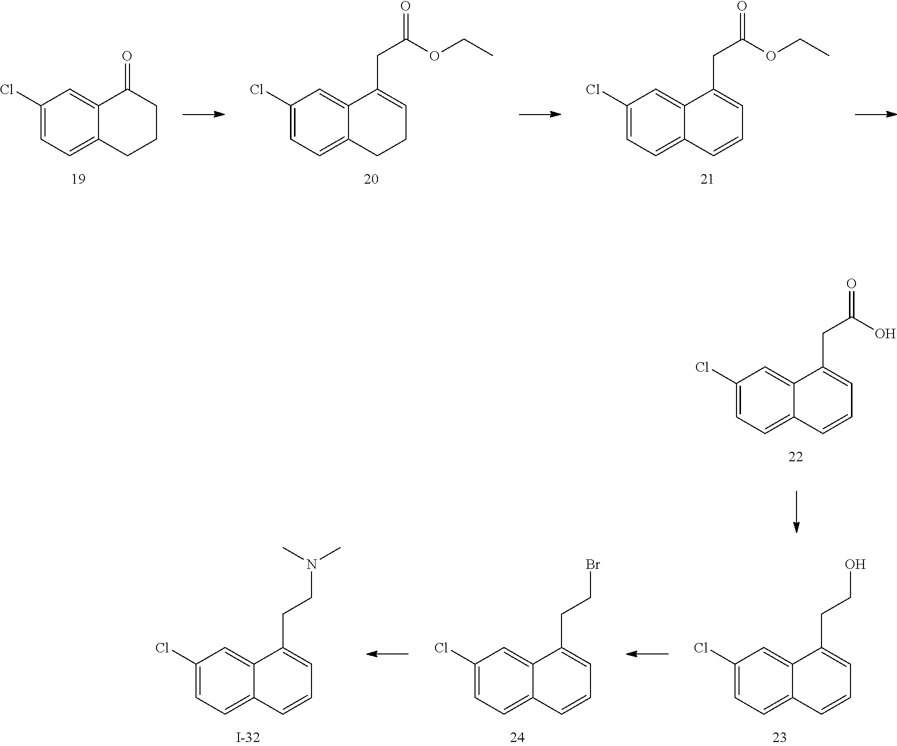

### Step 1: Ethyl 2-(7-chloro-3,4-dihydronaphthalen-1-yl)acetate (20)

An ice-cold solution of 7-chloro-1,2,3,4-tetrahydro-1-naphthalenone (3.60 g, 19.9 mmol) and NaOEt (1.49 g, 21.9 mmol) in dry EtOH (25 mL) was treated with a solution of ethyl (diethoxyphosphoryl)acetate (4.91 g, 21.9 mmol) in dry EtOH (10 mL) dropwise over 10 min. The reaction mass was warmed to RT and stirring continued for 4 h at which point the reaction was quenched by addition of H₂O (10 mL) and concentrated under reduced pressure. The resultant oil was diluted with H₂O (25 mL) and extracted with EtOAc (3×25 mL). The combined organic extracts were washed with brine (50 mL) before being dried (MgSO₄) and concentrated under reduced pressure. The crude oil was purified by column chromatography (SiO₂, 10-40% EtOAc/Hexanes (v/v)) to give the title compound as a colourless oil (4.1 g, 82%). ¹H NMR (400 MHz, CDCl₃) δ 7.18 (d, J=2.1 Hz, 1H), 7.13 (dd, J=8.0, 2.1 Hz, 1H), 7.07 (d, J=8.0 Hz, 1H), 6.07 (tt, J=4.6, 1.2 Hz, 1H), 4.19 (q, J=7.1 Hz, 2H), 3.42 (q, J=1.2 Hz, 2H), 2.77 (t, J=8.1 Hz, 2H), 2.36-2.31 (m, 2H), 1.28 (t, J=7.1 Hz, 3H). ¹³C NMR (101 MHz, CDCl₃) δ 171.5, 135.9, 134.6, 132.1, 130.5, 129.5, 128.7, 126.7, 122.9, 60.9, 39.0, 27.3, 23.1, 14.2.

### Step 2: Ethyl 2-(7-chloronaphthalen-1-yl)acetate (21)

DDQ (3.59 g, 15.8 mmol) was added portionwise to a stirred solution of ethyl (7-chloro-3,4-dihydro-1-naphthyl)acetate (3.3 g, 13.2 mmol) in CH₂Cl₂ (50 mL). Stirring was continued for 4 h at which time the reaction mass was filtered and the residue washed with Et₂O (3×20 mL). The combined filtrates were concentrated under reduced pressure and the resultant crude material purified by column chromatography to give ethyl (7-chloro-1-naphthyl)acetate (3.0 g, 92%) as a colourless oil. ¹H NMR (400 MHz, CDCl₃) δ 8.02 (d, J=2.0 Hz, 1H), 7.90-7.76 (m, 2H), 7.48-7.43 (m, 3H), 4.20 (q, J=7.1 Hz, 2H), 4.04 (s, 2H), 1.28 (t, J=7.1 Hz, 3H).

### Step 3: 2-(7-chloronaphthalen-1-yl)acetic acid (22)

A solution of LiOH (867 mg, 36.2 mmol) in H₂O (10 mL) was added to a suspension of ethyl (7-chloro-1-naphthyl)acetate (3.0 g, 12.1 mmol) and TBAB (0.12 mmol) in THF (10 mL). Stirring was continued at RT for 12 h at which point the solution was concentrated under reduced pressure and washed with Et₂O (2×25 mL). The aqueous layer was made acidic by addition of conc. HCl (32% aq. soln.) and the resultant suspension extracted with CH₂Cl₂ (3×25 mL). The combined organic extracts were washed with H₂O (50 mL), followed by brine (50 mL) before being dried (MgSO₄) filtered and concentrated under reduced pressure to give the title compound as a white crystalline solid (2 g, 96%). ¹H NMR (400 MHz, CDCl₃) δ 7.97 (d, J=2.0 Hz, 1H), 7.86-7.76 (m, 2H), 7.50-7.41 (m, 3H), 4.07 (s, 2H).

### Step 4: 2-(7-chloronaphthalen-1-yl)ethan-1-ol (23)

A stirred solution of (7-chloro-1-naphthyl)acetic acid (2.22 g, 10.1 mmol) at 0° C. in THF (20 mL) was treated with LiAlH₄ (1.53 g, 40 mmol) and the resultant suspension allowed to warm to RT. Stirring was continued for 6 h at this temperature at which point the reaction mass was cooled to 0° C. and treated sequentially with H₂O (1.5 mL), NaOH (15% aq. soln., 1.5 mL) and H₂O (4.5 mL) before being filtered through a pad of celite. The residue was washed with THF (3×50 mL), and the combined filtrates concentrated under reduced pressure. The crude oil thus obtained was purified by column chromatography (SiO₂, 10-40% EtOAc/Hexane (v/v)) to give the desired compound as a colourless oil (1.7 g, 82%). ¹H NMR (400 MHz, CDCl₃) δ 8.02 (d, J=2.0 Hz, 1H), 7.79 (d, J=8.7 Hz, 1H), 7.75-7.70 (m, 1H), 7.51-7.34 (m, 3H), 3.98 (t, J=6.7 Hz, 3H), 3.30 (t, J=6.7 Hz, 2H).

### Step 5: 1-(2-bromoethyl)-7-chloronaphthalene (24)

To a flask containing triphenylphosphine (3.95 g, 15.1 mmol), under N₂ atmosphere, was added a solution of 2-(7-chloro-1-naphthyl)ethan-1-ol (2.08 g, 10.1 mmol) and CBr₄ (2.12 g, 6.39 mmol) in dry CH₂Cl₂ (17.4 mL). The obtained mixture was stirred at 40° C. for 18 h. Et₂O (29 mL) was added and the resulting suspension was stirred at room temperature for 30 min. The brown precipitate formed was filtered and washed with Et₂O. The filtrates were concentrated under reduced pressure and the crude product was purified by flash chromatography (SiO₂, 0-5% EtOAc/Hexanes (v/v)) to give 1-(2-bromoethyl)-7-chloronaphthalene (2.21 g, 82%) as a colourless oil. ¹H NMR (400 MHz, CDCl₃) δ 7.98 (d, J=2.0 Hz, 1H), 7.84 (d, J=8.7 Hz, 1H), 7.78 (dd, J=7.6, 2.0 Hz, 1H), 7.50-7.39 (m, 3H), 3.70 (ddd, J=7.9, 6.9, 1.4 Hz, 2H), 3.61 (ddd, J=8.8, 6.9, 1.3 Hz, 2H). ¹³C NMR (101 MHz, CDCl₃) δ 134.2, 132.4, 132.3, 132.2, 130.6, 128.0, 127.6, 126.7, 125.8, 122.3, 36.4, 31.7.

### Step 6: 2-(7-chloronaphthalen-1-yl)-N,N-dimethylethan-1-amine (I-32·HCl)

A suspension of 1-(2-bromoethyl)-7-chloronaphthalene (150 mg, 0.56 mmol) and K₂CO₃ (154 mg, 1.11 mmol) was treated with a solution of 2 M Me₂NH in THF (620 μL, 1.23 mmol) at RT. The reaction mass was then heated to 70° C. for 8 h before being diluted with H₂O (15 mL) and extracted with EtOAc (3×25 mL). The combined organic extracts were then washed sequentially with H₂O (3×25 mL), brine (50 mL) before being dried (MgSO₄), filtered and concentrated under reduced pressure. The resultant oil was purified by column chromatography (SiO₂, 1-10% MeOH/NH₃ in CH₂Cl₂) to give the title compound as a colourless oil (85 mg). This oil was converted to the HCl salt following General Procedure A which resulted in crystalline needles that were collected by filtration, air dried, and identified as the hydrochloride salt of the title compound (I-32·HCl, 0.1 g, 67%). HPLC Purity: 99.4% (254 nm); ¹H NMR (400 MHz, DMSO-d₆) δ 10.88-10.83 (m, 1H), 8.29 (d, J=1.9 Hz, 1H), 8.02 (d, J=8.8 Hz, 1H), 7.95-7.86 (m, 1H), 7.57 (dd, J=8.7, 2.0 Hz, 1H), 7.54-7.51 (m, 2H), 3.54-3.41 (m, 2H), 3.36-3.27 (m, 2H), 2.88 (d, J=4.9 Hz, 6H). ¹³C NMR (101 MHz, DMSO-d₆) δ 132.7, 132.1, 131.9, 131.5, 130.9, 128.0, 127.5, 126.6, 126.2, 122.6, 56.4, 42.0, 26.9.

### Example 33: Synthesis of N-ethyl-N-methyl-2-(naphthalen-1-yl)ethan-1-amine (I-33)

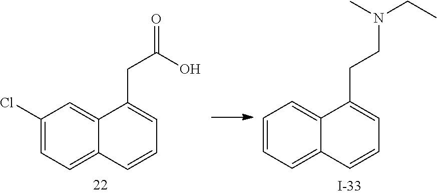

### Step 1: N-ethyl-N-methyl-2-(naphthalen-1-yl)ethan-1-amine (I-33·Fum)

A stirred solution of (7-chloro-1-naphthyl)acetic acid (0.5 g, 2.27 mmol) in CH₂Cl₂ (10 mL) was treated with oxalyl chloride (0.23 mL) followed by a drop of DMF. Stirring was continued for 1 h at which point the reaction was concentrated under a stream of N₂ gas. The resultant yellow oil was then re-dissolved in CH₂Cl₂ (10 mL) and cooled to 0° C. before being treated with N-methylethylamine (201 mg, 3.4 mmol) followed by dropwise addition of triethylamine (0.79 mL, 5.66 mmol) and stirring continued for 3 h. The reaction was then diluted with H₂O (25 mL) and the phases separated. The organic phase was washed with NaHCO₃ (sat. aq. soln., 25 mL) followed by brine before being dried (MgSO₄), filtered and concentrated under reduced pressure. The resultant residue was triturated with Et₂O (10 mL) and filtered to give a white powder that was dissolved in anhydrous THF (25 mL), cooled to 0° C. before being treated with LiAlH₄ (166 mg, 4.38 mmol) portionwise and the resultant suspension was then heated at reflux for 4 h. The reaction was then cooled to 0° C. and quenched by sequential dropwise addition of H₂O (0.15 mL), NaOH (0.15 mL, 15% aq. soln.) and H₂O (1 mL) followed by the addition of MgSO₄ (1 g). The suspension was then filtered through a pad of celite and the residue washed with THF (2×25 mL). The combined filtrates were concentrated under reduced pressure and the crude oil purified by flash column chromatography (SiO₂, 0-5%, MeOH/NH₃ in CH₂Cl₂ (v/v)) to give the title compound as a colourless oil (85 mg). This free base was converted to the fumarate salt as per General Procedure B resulting in a white crystalline product that was identified as the title compound (I-33·Fum, 0.1 g, 80%). HPLC Purity: 96.0% (254 nm); ¹H NMR (400 MHz, DMSO-d₆) δ 8.13 (ddd, J=7.9, 1.7, 0.7 Hz, 1H), 7.99-7.90 (m, 1H), 7.90-7.77 (m, 1H), 7.62-7.49 (m, 2H), 7.49-7.40 (m, 2H), 6.57 (s, 2H), 3.40-3.31 (m, 2H), 3.03-2.92 (m, 2H), 2.87 (q, J=7.2 Hz, 2H), 2.59 (s, 3H), 1.14 (t, J=7.2 Hz, 3H). ¹³C NMR (101 MHz, DMSO-d₆) δ 167.9, 135.4, 135.2, 133.9, 131.8, 129.1, 127.5, 127.3, 126.7, 126.2, 126.1, 124.0, 56.5, 50.5, 39.9, 28.7, 10.8.

### Example 34: Synthesis of N-methyl-N-(2-(naphthalen-1-yl)ethyl)propan-2-amine (I-34)

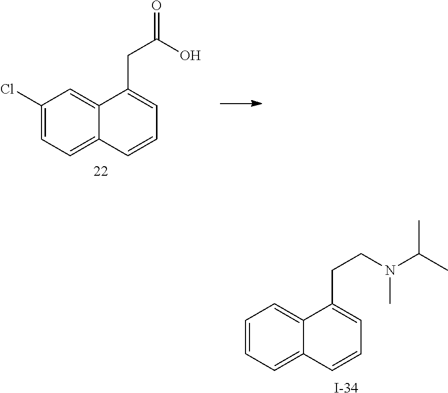

### Step 1: N-methyl-N-(2-(naphthalen-1-yl)ethyl)propan-2-amine hydrochloride (I-34·HCl)

A stirred solution of (7-chloro-1-naphthyl)acetic acid (0.5 g, 2.27 mmol) in CH₂Cl₂ (10 mL) was treated with oxalyl chloride (0.23 mL) followed by a drop of DMF. Stirring was continued for 1 h at which point the reaction was concentrated under a stream of N₂ gas. The resultant yellow oil was then re-dissolved in CH₂Cl₂ (10 mL) and cooled to 0° C. before being treated with N-methylisopropylamine (199 mg, 2.72 mmol) followed by dropwise addition of triethylamine (0.79 mL, 5.66 mmol) and stirring continued for 3 h. The reaction was then diluted with H₂O (25 mL) and the phases separated. The organic phase was washed with NaHCO₃ (sat. aq. soln., 25 mL) followed by brine before being dried (MgSO₄), filtered and concentrated under reduced pressure. The resultant residue was triturated with Et₂O (10 mL) and filtered to give a white powder that was dissolved in anhydrous THF (25 mL), cooled to 0° C. before being treated with LiAlH₄ (166 mg, 4.38 mmol) portionwise and the resultant suspension was then heated at reflux for 4 h. The reaction was then cooled to 0° C. and quenched by sequential dropwise addition of H₂O (0.15 mL), NaOH (0.15 mL, 15% aq. soln.) and H₂O (1 mL) followed by the addition of MgSO₄ (1 g). The suspension was then filtered through a pad of celite and the residue washed with THF (2×25 mL). The combined filtrates were concentrated under reduced pressure and the crude oil purified by flash column chromatography (SiO₂, 0-5%, MeOH/NH₃ in CH₂Cl₂ (v/v)) to give the title compound as a colourless. This oil was converted to the HCl salt as per General Procedure A, and the resultant crystals were collected, washed with cold acetone (3 mL) followed by Et₂O (5 mL) and air dried to give the title compound as the HCl salt (172 mg, 25%). HPLC Purity: 93% (254 nm); ¹H NMR (400 MHz, DMSO-d₆): δ 10.93 (s, 1H), 8.29 (d, J=8.3 Hz, 1H), 8.03-7.92 (m, 1H), 7.85 (dd, J=6.9, 2.5 Hz, 1H), 7.67-7.52 (m, 2H), 7.52-7.43 (m, 2H), 3.78-3.58 (m, 2H), 3.52 (td, J=12.5, 5.8 Hz, 1H), 3.31-3.13 (m, 2H), 2.77 (d, J=5.1 Hz, 3H), 1.32 (d, J=6.6 Hz, 3H), 1.23 (d, J=6.6 Hz, 3H); ¹³C NMR (101 MHz, DMSO-d₆) δ 134.0, 133.9, 131.8, 129.2, 128.0, 127.5, 127.0, 126.4, 126.1, 124.2, 56.3, 53.0, 34.4, 27.6, 17.4, 15.3.

### Example 35: Synthesis of N-(2-(7-chloronaphthalen-1-yl)ethyl)-N-methylcyclopropanamine (I-35)

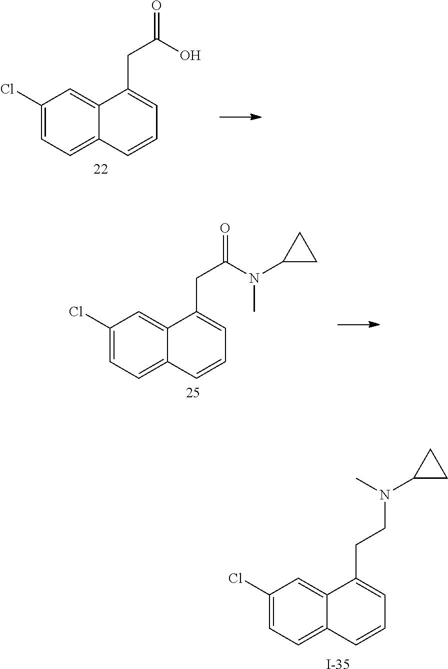

### Step 1: 2-(7-chloronaphthalen-1-yl)-N-cyclopropyl-N-methylacetamide (25)

A stirred solution of (7-chloro-1-naphthyl)acetic acid (0.5 g, 2.27 mmol) in CH₂Cl₂ (10 mL) was treated with oxalyl chloride (0.23 mL) followed by a drop of DMF. Stirring was continued for 1 h at which point the reaction was concentrated under a stream of N₂ gas. The resultant yellow oil was then re-dissolved in CH₂Cl₂ (10 mL) and cooled to 0° C. before being treated with N-methylcyclopropylamine hydrochloride (293 mg, 2.72 mmol) followed by dropwise addition of triethylamine (0.79 mL, 5.66 mmol) and stirring continued for 3 h. The reaction was then diluted with H₂O (25 mL) and the phases separated. The organic phase was washed with NaHCO₃ (sat. aq. soln., 25 mL) followed by brine before being dried (MgSO₄), filtered and concentrated under reduced pressure. The resultant solid was purified by flash column chromatography (SiO₂, 0-50%, EtOAc/Hex (v/v)) to give the title compound as a white solid (275 mg, 44%). ¹H NMR (400 MHz, CDCl₃) δ 7.93 (d, J=2.0 Hz, 1H), 7.81 (d, J=8.7 Hz, 1H), 7.76 (d, J=8.0 Hz, 1H), 7.57-7.37 (m, 3H), 4.29 (s, 2H), 3.02 (s, 3H), 2.81-7.30 (m, 1H), 0.97-0.86 (m, 4H).

### Step 2: N-(2-(7-chloronaphthalen-1-yl)ethyl)-N-methylcyclopropanamine (I-35-mal)

A stirred solution of 2-(7-chloronaphthalen-1-yl)-N-cyclopropyl-N-methylacetamide (0.3 g, 1.10 mmol) in THF (10 mL) at 0° C. under an inert atmosphere was treated with LiAlH₄ (166 mg, 4.38 mmol) portionwise and the resultant suspension was then heated at reflux for 4 h. The reaction was then cooled to 0° C. and quenched by sequential dropwise addition of H₂O (0.15 mL), NaOH (0.15 mL, 15% aq. soln.) and H₂O (1 mL) followed by the addition of MgSO₄ (1 g). The suspension was then filtered through a pad of celite and the residue washed with THF (2×25 mL). The combined filtrates were concentrated under reduced pressure and the crude oil purified by flash column chromatography (SiO₂, 0-5%, MeOH/NH₃ in CH₂Cl₂ (v/v)) to give the title compound as a colourless oil (174 mg). The resultant oil was taken up in warm acetone (1 mL) and added to a boiling solution of maleic acid (134 mg, 1.15 mmol) in acetone (15 mL) and allowed to cool to 0° C. overnight. The white crystalline precipitate was collected by suction filtration, and the residue washed with cold Et₂O (2×10 mL) and air dried yielding the title compound as the maleate salt (I-35·mal, 112 mg, 27%). HPLC Purity: 95% (280 nm); ¹H NMR (400 MHz, DMSO-d₆) δ 8.17 (d, J=2.1 Hz, 1H), 8.04 (d, J=8.8 Hz, 1H), 7.92 (dq, J=8.1, 4.0 Hz, 1H), 7.59 (dd, J=8.8, 2.1 Hz, 1H), 7.56-7.51 (m, 2H), 6.10 (s, 2H), 3.47 (s, 4H), 2.99 (s, 4H), 1.23-0.66 (m, 4H). ¹³C NMR (101 MHz, DMSO-d₆) δ 167.6, 135.1, 133.2, 132.4, 132.4, 131.9, 131.5, 128.6, 128.0, 127.0, 126.7, 122.8, 57.0, 41.7, 39.0, 27.5, 5.0.

### Example 37: Synthesis of N-benzyl-2-(naphthalen-1-yl)ethan-1-amine (I-37)

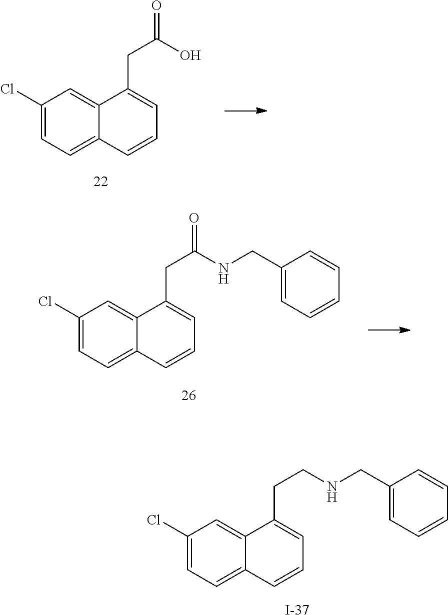

### Step 1: N-benzyl-2-(7-chloronaphthalen-1-yl)acetamide (26)

A stirred solution of (7-chloro-1-naphthyl)acetic acid (0.5 g, 2.27 mmol) in CH₂Cl₂ (10 mL) was treated with oxalyl chloride (0.23 mL) followed by a drop of DMF. Stirring was continued for 1 h at which point the reaction was concentrated under a stream of N₂ gas. The resultant yellow oil was then re-dissolved in CH₂Cl₂ (10 mL) and cooled to 0° C. before being treated with benzylamine (364 mg, 3.40 mmol) followed by dropwise addition of triethylamine (0.79 mL, 5.66 mmol) and stirring continued for 3 h. The reaction was then diluted with H₂O (25 mL) and the phases separated. The organic phase was washed with NaHCO₃ (sat. aq. soln., 25 mL) followed by brine before being dried (MgSO₄), filtered and concentrated under reduced pressure. The resultant solid was purified by flash column chromatography (SiO₂, 0-50% EtOAc/Hexane (v/v)) to give the title compound as a white solid (410 mg, 57%). ¹H NMR (400 MHz, DMSO-d₆) δ 8.73 (t, J=6.0 Hz, 1H), 8.20 (d, J=2.1 Hz, 1H), 7.99 (d, J=8.7 Hz, 1H), 7.88 (dd, J=7.3, 2.1 Hz, 1H), 7.58-7.46 (m, 3H), 7.35-7.19 (m, 5H), 4.30 (d, J=5.9 Hz, 2H), 3.98 (s, 2H).

### Step 2: N-benzyl-2-(naphthalen-1-yl)ethan-1-amine (I-37·HCl)

A stirred solution of N-benzyl-2-(7-chloronaphthalen-1-yl)acetamide (0.35 g, 1.13 mmol) in THF (10 mL) at 0° C. under an inert atmosphere was treated with LiAlH₄ (172 mg, 4.52 mmol) portionwise and the resultant suspension was then heated at reflux for 4 h. The reaction was then cooled to 0° C. and quenched by sequential dropwise addition of H₂O (0.15 mL), NaOH (0.15 mL, 15% aq. soln.) and H₂O (1 mL) followed by the addition of MgSO₄ (1 g). The suspension was then filtered through a pad of celite and the residue washed with THF (2×25 mL). The combined filtrates were concentrated under reduced pressure and the crude oil purified by flash column chromatography (SiO₂, 0-5%, MeOH/NH₃ in CH₂Cl₂ (v/v)) to give the title compound as a colourless oil (114 mg). The resultant oil was converted to the HCl salt according to General Procedure A. The white crystalline precipitate was collected by suction filtration, and the residue washed with cold Et₂O (2×10 mL) and air dried yielding the title compound as the HCl salt (I-37·HCl, 60 mg, 16%). HPLC Purity: 92% (280 nm); ¹H NMR (400 MHz, DMSO-d₆) δ 9.73-9.61 (br. s, 2H), 8.24-8.18 (m, 1H), 8.01-7.93 (m, 1H), 7.86 (dd, J=8.1, 1.3 Hz, 1H), 7.67-7.52 (m, 4H), 7.56-7.44 (m, 2H), 7.49-7.38 (m, 4H), 4.22 (t, J=5.9 Hz, 2H), 3.57-3.48 (m, 2H), 3.25-3.15 (m, 2H). ¹³C NMR (101 MHz, DMSO-d₆) δ 134.0, 133.8, 132.6, 131.7, 130.5, 129.4, 129.2, 129.1, 128.0, 127.4, 126.9, 126.4, 126.2, 124.0, 50.3, 47.6, 29.3.

Scheme 8: Compounds of general formula (I) can be synthesised from an appropriately substituted cyclic ketone following the outlined sequence of steps in Scheme 8 or similar as one skilled in the art may utilise. Alternatively, as depicted in Scheme 8, a 3,4-dihydronaphthalen-1(2H)-one with the appropriate aromatic substitution pattern can be reacted with cyanoacetic acid, which yields the corresponding substituted cyclohexene after elimination. The substituted cyclohexene can be oxidised with various oxidants, for example, 2,3-dichloro-5,6-dicyano-p-benzoquinone (DDQ) to the corresponding 6,6-aromatic system. The nitrile can be reduced to give the primary amine which can then be subjected to reductive N-alkylation with the appropriate aldehyde to give compounds of general formula (I) (exemplified by Example I-38, I-42, I-50, I-52, and I-53).

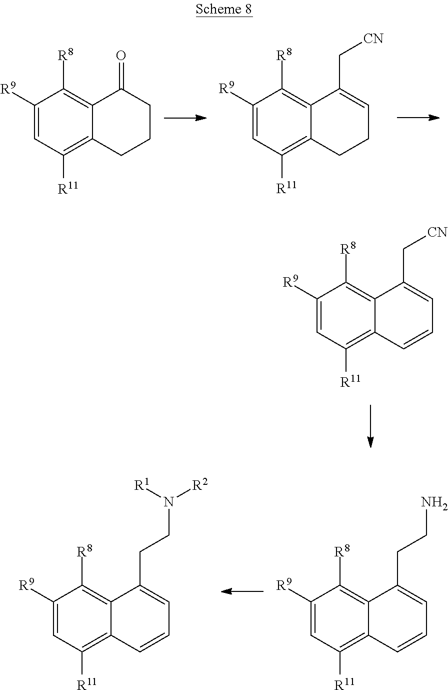

### Example 53: Synthesis of 2-(7-fluoronaphthalen-1-yl)ethan-1-amine (I-53)

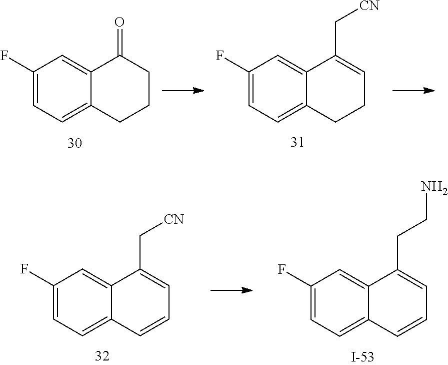

### Step 1: 2-(7-Fluoro-3,4-dihydronaphthalen-1-yl)acetonitrile (31)

To a solution of 7-fluoro-3,4-dihydronaphthalen-1(2H)-one (5.0 g, 30.5 mmol) in toluene (50 mL) was added cyanoacetic acid (3.89 g, 45.7 mmol), heptanoic acid (1.08 mL, 7.61 mmol) and benzylamine (0.83 mL, 7.61 mmol). The reaction was then refluxed at 135° C. for 16 h attached to a Dean-Stark apparatus and then cooled to RT upon completion. The reaction was diluted with EtOAc (100 mL), washed with 0.5 M aq. NaOH (50 mL), then saturated aq. NaHCO₃ (100 mL), 1 M aq. HCl (100 mL), followed by brine (50 mL). The organic layer was dried over anhydrous Na₂SO₄, filtered, and the filtrate concentrated. The solid residue was recrystallised from isopropanol with a few drops of H₂O and stood in the fridge for 16 h to afford the title compound as off-white crystals (4.6 g, 81%). ¹H NMR (400 MHz, DMSO-d₆) δ 7.23 (dd, J=8.2, 6.1 Hz, 1H), 7.11 (dd, J=10.5, 2.6 Hz, 1H), 7.03 (td, J=8.6, 2.6 Hz, 1H), 6.26 (t, J=4.4 Hz, 1H), 3.94-3.72 (m, 2H), 2.70 (t, J=8.1 Hz, 2H), 2.36-2.17 (m, 2H). ¹³C NMR (101 MHz, DMSO-d₆) δ 161.2 (d, J=240.4 Hz), 134.3 (d, J=7.8 Hz), 131.8 (d, J=3.0 Hz), 129.7, 129.1 (d, J=8.1 Hz), 126.5 (d, J=2.3 Hz), 118.7, 113.7 (d, J=20.9 Hz), 109.8 (d, J=23.0 Hz), 26.2, 22.6, 20.5.

### Step 2: 2-(7-Fluoronaphthalen-1-yl)acetonitrile (32)

A sealed pressure tube containing (7-fluoro-3,4-dihydro-1-naphthyl)acetonitrile (1.0 g, 5.34 mmol) and DDQ (98% purity, 1.24 g, 5.34 mmol) in CH₂Cl₂ (26 mL) was heated to 60° C. for 2 h. The cooled reaction mixture was filtered through a silica plug and eluted with CH₂Cl₂. The combined filtrate was concentrated under a stream of N₂ gas and the residue was purified by flash chromatography (0% to 40% CH₂Cl₂ in hexane) to afford the title compound as orange crystals (570 mg, 58%). ¹H NMR (400 MHz, DMSO-d₆) δ 8.10 (dd, J=9.0, 6.1 Hz, 1H), 7.98 (d, J=8.3 Hz, 1H), 7.82 (dd, J=11.3, 2.5 Hz, 1H), 7.65 (d, J=7.1 Hz, 1H), 7.56-7.43 (m, 2H), 4.47 (s, 2H). ¹³C NMR (101 MHz, DMSO-d₆) δ 160.5 (d, J=244.6 Hz), 131.7 (d, J=9.3 Hz), 131.5 (d, J=9.1 Hz), 130.5, 128.5, 127.4, 127.1 (d, J=5.6 Hz), 125.1 (d, J=2.5 Hz), 118.9, 116.4 (d, J=25.2 Hz), 107.3 (d, J=21.8 Hz), 20.6.

### Step 3: 2-(7-fluoronaphthalen-1-yl)ethan-1-amine hydrochloride (I-53·HCl)

An ice-cold solution of (7-fluoro-1-naphthyl)acetonitrile (500 mg, 2.70 mmol) in anhydrous THF (20 mL) was treated with LiAlH₄ (205 mg, 5.40 mmol) in portions and the resulting suspension was stirred at reflux for 16 h. The reaction was then cooled in an ice-bath and treated with additional LiAlH₄ (300 mg, 8.10 mmol) and refluxed for a further 8 h. The reaction was then cooled in an ice-bath and quenched with H₂O (0.8 mL), 15% aq. NaOH (0.8 mL), and H₂O (2.4 mL). The suspension was then stirred with anhydrous Na₂SO₄ for 30 mins before being filtered through a celite plug and the filter cake washed with several portions of hot THF. The combined filtrate was concentrated in vacuo and the residue was purified by flash chromatography (0.1% to 5% MeOH/NH₃ in CH₂Cl₂) to afford the title compound as a yellow oil. A solution of this oil in CHCl₃ (50 mL) and iPrOH (1 mL) was made acidic by addition of HCl (35% aq. soln.) and Et₂O added until a lasting turbidity which upon cooling for 18 h at 0° C. formed the title compound as the hydrochloride salt which were white crystals (250 mg, 41%). ¹H qNMR purity: 98.4% (ERETIC); ¹H NMR (400 MHz, DMSO-d₆) δ 8.23 (br. s, 3H), 8.05 (dd, J=9.0, 6.1 Hz, 1H), 7.96 (dd, J=11.6, 2.5 Hz, 1H), 7.89 (d, J=7.7 Hz, 1H), 7.56-7.36 (m, 3H), 3.41-3.29 (m, 2H), 3.14-2.99 (m, 2H). ¹³C NMR (101 MHz, DMSO-d₆) δ 160.5 (d, J=243.6 Hz), 133.2 (d, J=5.7 Hz), 132.3 (d, J=8.8 Hz), 131.7 (d, J=9.3 Hz), 130.7, 128.1, 127.4, 125.1 (d, J=2.2 Hz), 116.0 (d, J=25.3 Hz), 107.3 (d, J=21.3 Hz), 39.4, 30.2.

### Example 38: Synthesis of 2-(7-fluoronaphthalen-1-yl)-N,N-dimethylethan-1-amine (I-38)

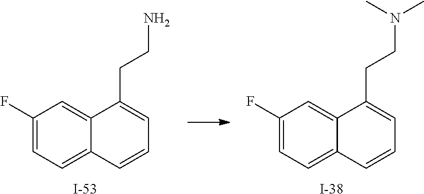

### Step 1: 2-(7-fluoronaphthalen-1-yl)-N,N-dimethylethan-1-amine (I-38)

A solution of 2-(7-fluoro-1-naphthyl)ethylamine hydrochloride (140 mg, 0.62 mmol) and aq, formaldehyde (37% w/w, 0.23 mL, 3.10 mmol) in 1,2-dichloroethane (5 mL) was treated with NaBH(OAc)₃ (197 mg, 0.93 mmol) at RT. The reaction was stirred at RT for 1 h and a further portion of NaBH(OAc)₃ (197 mg, 0.93 mmol) was added and stirring continued for 1 h. The reaction was diluted with CH₂Cl₂ (20 mL) and then quenched with 1 M aq. NaOH (10 mL). The mixture was then extracted with CH₂Cl₂ (20 mL×3) and the combined organic layers were washed with brine (50 mL) and then concentrated under reduced pressure. The residue was purified by flash chromatography (0.1% to 6% MeOH/NH₃ in CH₂Cl₂) to afford the title compound as a yellow oil (90 mg, 67%). ¹H NMR (400 MHz, DMSO-d₆) δ 8.05 (dd, J=9.0, 6.1 Hz, 1H), 7.99-7.86 (m, 2H), 7.55-7.40 (m, 3H), 3.51-3.14 (m, 4H), 2.80 (s, 6H). ¹³C NMR (101 MHz, DMSO-d₆) δ 160.5 (d, J=243.7 Hz), 133.1 (d, J=5.8 Hz), 132.2 (d, J=8.8 Hz), 131.7 (d, J=9.4 Hz), 130.7, 127.9, 127.4, 125.1 (d, J=2.2 Hz), 116.0 (d, J=25.2 Hz), 107.4 (d, J=21.3 Hz), 56.8, 42.4, 27.6.

### Step 2: 2-(7-fluoronaphthalen-1-yl)-N,N-dimethylethan-1-amine fumarate (I-38·Fum)

2-(7-fluoronaphthalen-1-yl)-N,N-dimethylethan-1-amine (135 mg, 0.62 mmol) was formulated as the fumarate salt according to general procedure B which provided the title compound as off-white crystals (65 mg, 31%). ¹H NMR (400 MHz, DMSO-d₆) δ 8.03 (dd, J=9.0, 6.1 Hz, 1H), 7.89-7.80 (m, 2H), 7.49-7.39 (m, 3H), 6.57 (s, 2H), 3.34-3.23 (m, 2H), 2.96-2.86 (m, 2H), 2.54 (s, 6H). ¹³C NMR (101 MHz, DMSO-d₆) δ 167.2, 160.3 (d, J=243.4 Hz), 134.7, 134.7, 132.3 (d, J=8.7 Hz), 131.6 (d, J=9.3 Hz), 130.6, 127.7, 127.0, 125.0 (d, J=2.3 Hz), 115.8 (d, J=25.4 Hz), 107.3 (d, J=21.2 Hz), 58.1, 43.4, 28.7.

Scheme 9: Compounds of general formula (I) can be synthesised from an appropriately substituted cyclic ketone following the outlined sequence of steps in Scheme 8 or similar as one skilled in the art may utilise. Alternatively, as depicted in Scheme 9, a 3,4-dihydronaphthalen-1(2H)-one with the appropriate aromatic substitution pattern can be subjected to Horner-Wadsworth-Emmons reaction generating cyclohexene analogues that upon oxidation yield napthyl acetate intermediates. Ester hydrolysis generates napthylacetic acid derivatives which can be subjected to amidation reactions as described previously (Scheme 2). The resulting amides can be reduced utilising numerous methods as one skilled in the art may employ, including but not limited to, borane-dimethylsulfide complex or lithium aluminium hydride. This allows access to the appropriate amine compounds of general formula (I) (exemplified by Example I-39, I-40, and I-41).

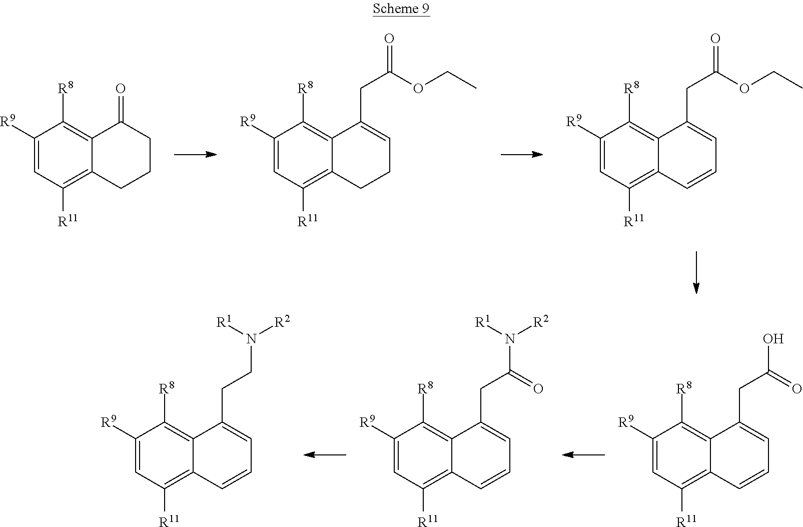

### Example 39: Synthesis of N-ethyl-2-(7-fluoronaphthalen-1-yl)-N-methylethan-1-amine (I-39)

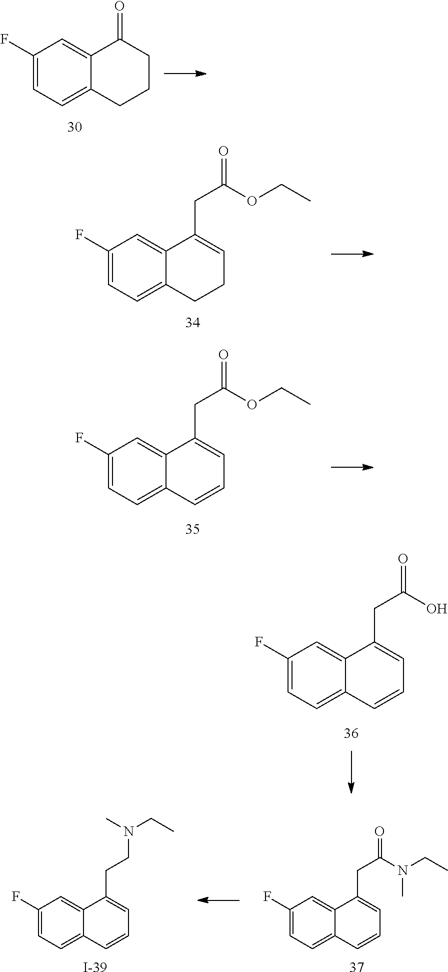

### Step 1: Ethyl 2-(7-fluoro-3,4-dihydronaphthalen-1-yl)acetate (34)

To a solution of ethyl 2-(diethoxyphosphoryl)acetate (5.47 mL, 27.6 mmol) in anhydrous EtOH (30 mL) was added sodium ethoxide (3.13 g, 45.9 mmol) and the mixture was stirred at room temperature for 10 min. A solution of 7-fluoro-3,4-dihydronaphthalen-1(2H)-one (3.77 g, 23 mmol) in anhydrous EtOH (40 mL) was added and the reaction was stirred at reflux under an inert atmosphere. At 4 h another pre-stirred mixture of ethyl 2-(diethoxyphosphoryl)acetate (2.27 mL) and sodium ethoxide (1.57 g) was added and the reaction was stirred at reflux for a further 3 h. The reaction was then cooled and concentrated under a stream of N₂ gas. The residue obtained was then treated with H₂O (40 mL) and subsequently extracted with EtOAc (3×50 mL). The combined organics were washed with brine (30 mL), dried over MgSO₄ and concentrated under reduced pressure. The residue was purified by flash chromatography (SiO₂, 0-5% EtOAc/Hexane) to afford the title compound (4.10 g, 76%) as a yellow oil. ¹H NMR (400 MHz, CDCl₃) δ 7.07 (ddt, J=8.0, 5.9, 1.0 Hz, 1H), 6.90 (dd, J=10.3, 2.6 Hz, 1H), 6.82 (td, J=8.4, 2.6 Hz, 1H), 6.08-6.03 (m, 1H), 4.16 (q, J=7.1 Hz, 2H), 3.39 (q, J=1.2 Hz, 2H), 2.79-2.70 (m, 2H), 2.36-2.26 (m, 2H), 1.24 (t, J=7.1 Hz, 3H). ¹³C NMR (101 MHz, CDCl₃) δ 171.7, 161.9 (d, J=242.3 Hz), 136.2 (d, J=7.4 Hz), 131.8 (d, J=3.0 Hz), 130.6, 129.8 (d, J=2.4 Hz), 128.7 (d, J=8.0 Hz), 113.3 (d, J=21.1 Hz), 110.1 (d, J=22.9 Hz), 61.0, 39.2, 27.3, 23.5, 14.3.

### Step 2: Ethyl 2-(7-fluoronaphthalen-1-yl)acetate (35)

A mixture of ethyl 2-(7-fluoro-3,4-dihydronaphthalen-1-yl)acetate (4.10 g, 17.5 mmol), 10% palladium on carbon (3.73 g, 3.5 mmol) and d-limonene (57 mL) were stirred at 180° C. for 2 h at which point violent gas evolution was observed. After cooling, the mixture was then diluted with EtOAc (100 mL) and filtered through a pad of celite. The filtrate was concentrated under reduced pressure and then purified by flash chromatography (SiO₂, 0-5% EtOAc/Hexane) to afford the title compound (1.6 g, 39%) as a yellow oil. ¹H NMR (400 MHz, CDCl₃): δ 7.85 (dd, J=9.0, 5.9 Hz, 1H), 7.80-7.75 (m, 1H), 7.62 (dd, J=11.2, 2.4 Hz, 1H), 7.46-7.36 (m, 2H), 7.27 (ddd, J=9.0, 8.2, 2.5 Hz, 1H), 4.17 (q, J=7.1 Hz, 2H), 4.00 (s, 2H), 1.24 (t, J=7.1 Hz, 3H).

### Step 4: 2-(7-fluoronaphthalen-1-yl)acetic acid (36)

A solution of ethyl 2-(7-fluoronaphthalen-1-yl)acetate (1.6 g, 6.88 mmol) in THF (15 mL) was treated with aqueous NaOH (10.3 mmol, 15 mL) and the mixture was stirred at 45° C. for 7 h. The mixture was concentrated under a stream of nitrogen and the remaining aqueous mixture was adjusted to pH 2 and then extracted with EtOAc (3×15 mL). The combined organics were washed with brine (15 mL), dried over anhydrous MgSO₄ and concentrated under reduced pressure to afford the title compound (1.21 g, 86%) as a yellow oil. ¹H NMR (400 MHz, CDCl₃): δ 7.86 (dd, J=9.0, 5.9 Hz, 1H), 7.80 (dt, J=8.0, 1.2 Hz, 1H), 7.58 (dd, J=11.0, 2.5 Hz, 1H), 7.46-7.43 (m, 1H), 7.42-7.36 (m, 1H), 7.28 (ddd, J=9.0, 8.2, 2.4 Hz, 1H), 4.03 (s, 2H).

### Step 4: N-ethyl-2-(7-fluoronaphthalen-1-yl)-N-methylacetamide (37)

To a solution of 2-(7-fluoronaphthalen-1-yl)acetic acid (420 mg, 2.06 mmol) in DMF (2.0 mL) at 0° C. was added a solution of 2-(3H-[1,2,3]triazolo[4,5-b]pyridin-3-yl)-1,1,3,3-tetramethylisouronium hexafluorophosphate (1.56 g, 4.11 mmol) in DMF (4.0 mL), iPr₂NEt (1.06 g, 8.23 mmol) and then MeNHEt (146 mg, 2.47 mmol) and the mixture was stirred at RT for 3 h. The reaction was then diluted with H₂O (30 mL) and then extracted with EtOAc (3×20 mL). The combined organics were washed with brine (20 mL), dried over anhydrous MgSO₄ and concentrated under reduced pressure. The crude material was purified by flash chromatography (SiO₂, 0-40% EtOAc/Hexane) to afford the title compound as a yellow oil (368 mg, 73%). ¹H NMR (400 MHz, CDCl₃): δ 7.85 (dd, J=9.0, 5.9 Hz, 1H), 7.80-7.71 (m, 1H), 7.56 (dd, J=11.7, 2.5 Hz, 1H), 7.42-7.33 (m, 2H), 7.27 (ddd, J=8.8, 8.1, 2.5 Hz, 1H), 4.06 (s, 2H), 3.35-3.55 (m, 2H), 3.01 (s, 3H), 1.16 (t, J=7.1 Hz, 3H).

### Step 5: N-ethyl-2-(7-fluoronaphthalen-1-yl)-N-methylethan-1-amine (I-39)

To a stirred solution of N-ethyl-2-(7-fluoronaphthalen-1-yl)-N-methylacetamide (368 mg, 1.5 mmol) in anhydrous THF (10 mL) was added 2 M borane-dimethylsulfide complex in THF (3.0 mL, 6.0 mmol) in a dropwise fashion and the mixture was stirred at 60° C. for 1 h. After cooling, the mixture was carefully treated with 6 M aq. HCl (2.0 mL) followed by MeOH (4.0 mL) and then stirred at reflux for 1 h. After cooling, volatiles were removed under a stream of N₂ gas and the remaining aqueous phase was extracted with Et₂O (2×10 mL) which was then discarded. The pH was adjusted to 14 with NaOH (15% aq. soln.) and then extracted with Et₂O (3×10 mL). The combined organics were washed with brine (15 mL), dried over anhydrous MgSO₄ and concentrated under reduced pressure. The crude material was purified by flash chromatography (SiO₂, 2-10% MeOH/NH₃ in CH₂Cl₂) to afford the title compound as a clear oil (219 mg, 63%). ¹H NMR (400 MHz, CDCl₃): δ 7.84 (dd, J=9.0, 5.9 Hz, 1H), 7.71 (dd, J=7.1, 2.4 Hz, 1H), 7.66 (dd, J=11.3, 2.5 Hz, 1H), 7.42-7.32 (m, 2H), 7.30-7.21 (m, 1H), 3.25-3.16 (m, 2H), 2.77-2.68 (m, 2H), 2.57 (q, J=7.2 Hz, 2H), 2.41 (s, 3H), 1.13 (t, J=7.2 Hz, 3H).

### Step 6: N-ethyl-2-(7-fluoronaphthalen-1-yl)-N-methylethan-1-amine fumarate (I-39·Fum)

N-ethyl-2-(7-fluoronaphthalen-1-yl)-N-methylethan-1-amine (219 mg, 0.95 mmol) was formulated as the fumarate salt according to general procedure B which provided the title compound as white crystals (117 mg, 36%). HPLC purity: 99.8% (254 nm); ¹H NMR (400 MHz, DMSO-d₆): δ 8.03 (dd, J=9.0, 6.1 Hz, 1H), 7.89-7.81 (m, 2H), 7.51-7.39 (m, 3H), 6.56 (s, 2H), 3.32-3.24 (m, 2H), 2.98-2.90 (m, 2H), 2.83 (q, J=7.2 Hz, 2H), 2.56 (s, 3H), 1.12 (t, J=7.2 Hz, 3H). ¹³C NMR (101 MHz, DMSO-d₆): δ 167.1, 160.3 (d, J=243.5 Hz), 134.8 (d, J=5.9 Hz), 134.6, 132.3 (d, J=8.8 Hz), 131.6 (d, J=9.4 Hz), 130.6, 127.7, 126.9, 125.0 (d, J=2.8 Hz), 115.8 (d, J=25.2 Hz), 107.3 (d, J=21.3 Hz), 55.9, 50.1, 39.6, 28.4, 10.5.

### Example 40: Synthesis of N-(2-(7-fluoronaphthalen-1-yl)ethyl)-N-methylpropan-2-amine (I-40)

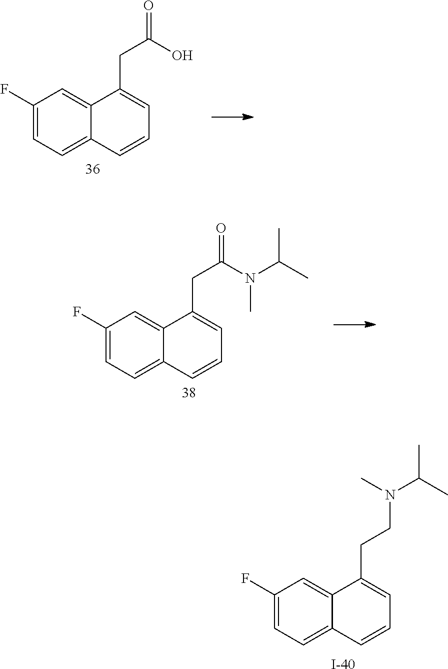

### Step 1: 2-(7-fluoronaphthalen-1-yl)-N-isopropyl-N-methylacetamide (38)

To a solution of 2-(7-fluoronaphthalen-1-yl)acetic acid (420 mg, 2.06 mmol) in DMF (2.0 mL) at 0° C. was added a solution of 2-(3H-[1,2,3]triazolo[4,5-b]pyridin-3-yl)-1,1,3,3-tetramethylisouronium hexafluorophosphate (1.56 g, 4.11 mmol) in DMF (4.0 mL), iPr₂NEt (1.06 g, 8.23 mmol) and then N-methyl(isopropyl)amine (181 mg, 2.47 mmol) and the mixture was stirred at room temperature for 3 h. The reaction was diluted with H₂O (30 mL) and then extracted with EtOAc (3×20 mL). The combined organics were washed with brine (20 mL), dried over anhydrous MgSO₄ and concentrated under reduced pressure. The crude material was purified by flash chromatography (SiO₂, 0-40% EtOAc/Hexane) to afford the title compound as a yellow oil (466 mg, 87%). ¹H NMR (400 MHz, CDCl₃): δ 7.85 (dd, J=9.0, 5.9 Hz, 1H), 7.76 (d, J=8.0 Hz, 1H), 7.54 (m, 1H), 7.37 (m, 2H), 7.32-7.22 (m, 1H), 5.02-4.92 (m, 1H), 4.08 (m, 2H), 2.85 (s, 3H), 1.13 (d, J=6.7 Hz, 6H).

### Step 2: N-(2-(7-fluoronaphthalen-1-yl)ethyl)-N-methylpropan-2-amine (I-40)

To a stirred solution of 2-(7-fluoronaphthalen-1-yl)-N-isopropyl-N-methylacetamide (466 mg, 1.8 mmol) in anhydrous THF (10 mL) was added 2 M borane-dimethylsulfide complex in THF (3.6 mL, 7.18 mmol) in a dropwise fashion and the mixture was stirred at 60° C. for 1 h. After cooling, the mixture was carefully treated with 6 M aq. HCl (2.0 mL) followed by MeOH (4.0 mL) and then stirred at reflux for 1 h. After cooling, volatiles were removed under a stream of N₂ gas and the remaining aqueous phase was extracted with Et₂O (2×10 mL) which was then discarded. The pH was adjusted to 14 with NaOH (1 M aq. soln.) and then extracted with Et₂O (3×10 mL). The combined organics were washed with brine (15 mL), dried over anhydrous MgSO₄ and concentrated under reduced pressure. The crude material was purified by flash chromatography (SiO₂, 2-10% MeOH/NH₃ in CH₂Cl₂) to afford the title compound as a clear oil (208 mg, 47%). ¹H NMR (400 MHz, CDCl₃): δ 7.84 (dd, J=9.0, 5.9 Hz, 1H), 7.72 (dd, J=7.8, 1.4 Hz, 1H), 7.67 (dd, J=11.3, 2.5 Hz, 1H), 7.43-7.32 (m, 2H), 7.31-7.21 (m, 1H), 3.29-3.20 (m, 2H), 3.10-2.96 (m, 1H), 2.82-2.73 (m, 2H), 2.44 (s, 3H), 1.09 (d, J=6.6 Hz, 6H).

### Step 3: N-(2-(7-fluoronaphthalen-1-yl)ethyl)-N-methylpropan-2-amine fumarate (I-40·Fum)

N-(2-(7-fluoronaphthalen-1-yl)ethyl)-N-methylpropan-2-amine (208 mg, 0.85 mmol) was formulated as the fumarate salt according to general procedure B which provided the title compound as white crystals (118 mg, 39%). HPLC purity: 99.2% (254 nm); ¹H NMR (400 MHz, DMSO-d₆): δ 8.03 (dd, J=9.0, 6.1 Hz, 1H), 7.92-7.83 (m, 2H), 7.53-7.40 (m, 3H), 6.57 (s, 3H), 3.43-3.26 (m, 3H), 3.02-2.96 (m, 2H), 2.57 (s, 3H), 1.11 (d, J=6.6 Hz, 6H); ¹³C NMR (101 MHz, DMSO-d₆): δ 166.9, 160.4 (d, J=243.5 Hz), 134.5, 132.3 (d, J=8.8 Hz), 131.6 (d, J=9.2 Hz), 130.6, 127.9, 127.0, 125.0 (d, J=2.1 Hz), 115.8 (d, J=25.2 Hz), 115.8 (d, J=25.2 Hz), 107.3 (d, J=21.3 Hz), 54.4, 53.0, 34.9, 28.8, 16.6.

### Example 41: Synthesis of N-(2-(7-fluoronaphthalen-1-yl)ethyl)-N-methylcyclopropanamine (I-41)

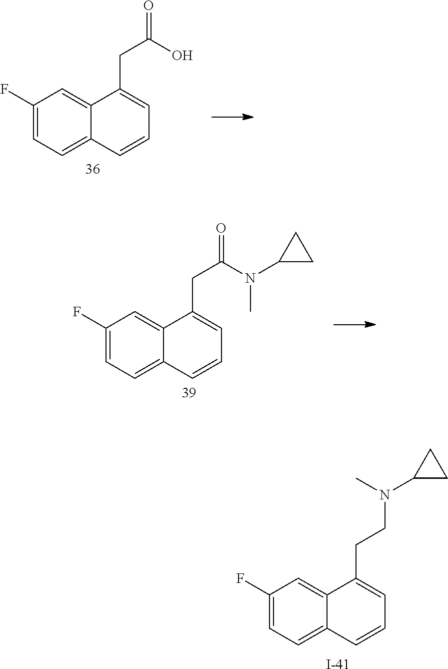

### Step 1: N-cyclopropyl-2-(7-fluoronaphthalen-1-yl)-N-methylacetamide (39)

To a solution of 2-(7-fluoronaphthalen-1-yl)acetic acid (420 mg, 2.06 mmol) in DMF (2.0 mL) at 0° C. was added in succession, a solution of 2-(3H-[1,2,3]triazolo[4,5-b]pyridin-3-yl)-1,1,3,3-tetramethylisouronium hexafluorophosphate (1.56 g, 4.11 mmol) in DMF (4.0 mL), and iPr₂NEt (1.06 g, 8.23 mmol), followed by a mixture of MeNHcPr hydrochloride (181 mg, 2.47 mmol) and iPr₂Net (398 mg, 3.08 mmol) in DMF (2.0 mL). The reaction mass was then stirred at RT for 3 h before being diluted with H₂O (30 mL) and extracted with EtOAc (3×20 mL). The combined organics were washed with brine (20 mL), dried over anhydrous MgSO₄ and concentrated under reduced pressure. The crude material was purified by flash chromatography (SiO₂, 0-40% EtOAc/Hexane) to afford the title compound as a yellow oil (371 mg, 70%). ¹H NMR (400 MHz, CDCl₃): δ 7.85 (dd, J=9.0, 5.9 Hz, 1H), 7.76 (m, 1H), 7.55 (dd, J=11.2, 2.5 Hz, 1H), 7.43-7.34 (m, 2H), 7.31-7.21 (m, 1H), 4.25 (s, 2H), 3.00 (s, 3H), 2.75 (tt, J=6.8, 4.1 Hz, 1H), 0.97-0.80 (m, 4H).

### Step 2: N-(2-(7-fluoronaphthalen-1-yl)ethyl)-N-methylcyclopropanamine (I-41)

To a stirred solution of N-cyclopropyl-2-(7-fluoronaphthalen-1-yl)-N-methylacetamide (371 mg, 1.44 mmol) in anhydrous THF (10 mL) was added 2 M borane-dimethylsulfide complex in THF (2.9 mL, 5.77 mmol) in a dropwise fashion and the mixture was stirred at 60° C. for 1 h. After cooling, the mixture was carefully treated with 6 M aq. HCl (2.0 mL) followed by MeOH (4.0 mL) and then stirred at reflux for 1 h. After cooling, volatiles were removed under a stream of N₂ gas and the remaining aqueous phase was extracted with Et₂O (2×10 mL) which was then discarded. The pH was adjusted to 14 with NaOH (1 M aq. soln.) and then extracted with Et₂O (3×10 mL). The combined organics were washed with brine (15 mL), dried over anhydrous MgSO₄ and concentrated under reduced pressure. The crude material was purified by flash chromatography (SiO₂, 2-10% MeOH/NH₃ in CH₂Cl₂) to afford the title compound as a clear oil (326 mg, 93%). ¹H NMR (400 MHz, CDCl₃) δ 7.84 (dd, J=9.0, 5.9 Hz, 1H), 7.71 (dd, J=7.4, 2.1 Hz, 1H), 7.67 (dd, J=11.4, 2.5 Hz, 1H), 7.42-7.32 (m, 2H), 7.30-7.21 (m, 1H), 3.32-3.23 (m, 2H), 2.97-2.88 (m, 2H), 2.54 (s, 3H), 1.87-1.78 (m, 1H), 0.58 (d, J=6.3 Hz, 4H).

### Step 3: N-(2-(7-fluoronaphthalen-1-yl)ethyl)-N-methylcyclopropanamine fumarate (I-41·Fum)

N-(2-(7-fluoronaphthalen-1-yl)ethyl)-N-methylcyclopropanamine (154 mg, 0.63 mmol) was formulated as the fumarate salt according to general procedure B which provided the title compound as white crystals (90 mg, 34%). HPLC purity: 99.9% (254 nm); ¹H NMR (400 MHz, DMSO-d₆): δ 7.84 (dd, J=9.0, 5.9 Hz, 1H), 7.71 (dd, J=7.4, 2.1 Hz, 1H), 7.67 (dd, J=11.4, 2.5 Hz, 1H), 7.42-7.32 (m, 2H), 7.30-7.21 (m, 2H), 3.32-3.23 (m, 2H), 2.97-2.88 (m, 2H), 2.54 (s, 3H), 1.87-1.78 (m, 1H), 0.58 (d, J=6.3 Hz, 4H); ¹³C NMR (101 MHz, DMSO-d₆): δ 166.1, 160.2 (d, J=243.3 Hz), 136.1 (d, J=5.7 Hz), 134.1, 132.3 (d, J=8.4 Hz), 131.6 (d, J=9.2 Hz), 130.6, 127.4, 126.5, 125.0 (d, J=2.8 Hz), 115.7 (d, J=25.1 Hz), 107.1 (d, J=21.2 Hz), 58.0, 42.0, 37.9, 29.6, 6.4.

### Example 42: Synthesis of N-benzyl-2-(7-fluoronaphthalen-1-yl)ethan-1-amine (I-42)

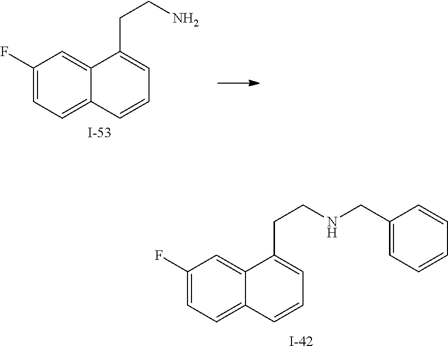

### Step 1: N-benzyl-2-(7-fluoronaphthalen-1-yl)ethan-1-amine (I-42)

To a stirred solution of 2-(7-fluoronaphthalen-1-yl)ethan-1-amine (75 mg, 0.40 mmol) in CH₂Cl₂ (3.0 mL) was added a solution of benzaldehyde (42.2 μL, 0.40 mmol) in CH₂Cl₂ (2.0 mL) followed by NaBH(OAc)₃ (101 mg, 0.48 mmol) and the mixture was stirred at room temperature for 2 h. The mixture was concentrated under a stream of N₂ gas and the residue treated with 1 M aq. NaOH (3.0 mL) which was extracted with Et₂O (3×5.0 mL). The combined organics were washed with brine (4.0 mL) dried over anhydrous MgSO₄ and concentrated under reduced pressure. The crude material was purified by flash chromatography (SiO₂, 2-10% MeOH/NH₃ in CH₂Cl₂) to provide the title compound (56 mg, 50%) as a clear oil. ¹H NMR (400 MHz, CDCl₃): δ 7.84 (dd, J=9.0, 5.9 Hz, 1H), 7.72 (dd, J=7.4, 1.8 Hz, 1H), 7.65 (dd, J=11.3, 2.5 Hz, 1H), 7.39-7.21 (m, 8H), 3.86 (s, 2H), 3.26 (t, J=7.4 Hz, 2H), 3.03 (t, J=7.4 Hz, 2H).

### Step 2: N-benzyl-2-(7-fluoronaphthalen-1-yl)ethan-1-amine fumarate (I-42·Fum)

N-benzyl-2-(7-fluoronaphthalen-1-yl)ethan-1-amine (56 mg, 0.20 mmol) was formulated as the fumarate salt according to general procedure B which provided the title compound as white crystals (51 mg, 65%). HPLC purity: 99.8% (254 nm); ¹H NMR (400 MHz, DMSO-d₆): δ 8.02 (dd, J=9.0, 6.1 Hz, 1H), 7.89-7.81 (m, 2H), 7.48-7.26 (m, 8H), 6.55 (s, 2H), 3.99 (s, 2H), 3.33-3.25 (m, 2H), 3.03-2.95 (m, 2H); ¹³C NMR (101 MHz, DMSO-d₆): δ 167.3, 160.4 (d, J=243.2 Hz), 134.8, 134.7, 132.3 (d, J=9.0 Hz), 136.5, 132.4, 131.6 (d, J=9.2 Hz), 130.6, 128.9, 128.4, 127.7 (d, J=4.4 Hz), 127.0, 125.0 (d, J=2.2 Hz), 115.8 (d, J=25.3 Hz), 107.3 (d, J=21.2 Hz), 51.2, 48.1, 30.9.

### Examples 52: Synthesis of 2-(5-methoxynaphthalen-1-yl)ethan-1-amine (I-52)

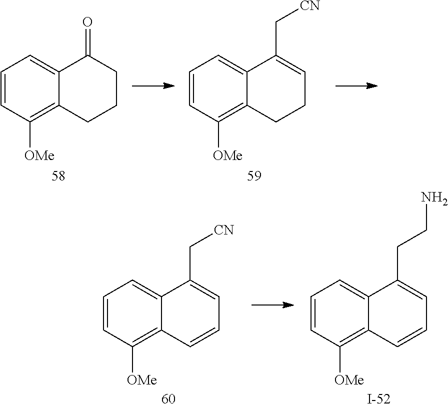

### Step 1: 2-(5-methoxy-3,4-dihydronaphthalen-1-yl)acetonitrile (59)

To a solution of 5-methoxy-3,4-dihydronaphthalen-1(2H)-one (8.0 g, 45.4 mmol) in toluene (60 mL) was added cyanoacetic acid (4.49 mL, 68.1 mmol), heptanoic acid (1.61 mL, 11.3 mmol) and benzylamine (1.24 mL, 11.3 mmol), and the resulting mixture was stirred at 135° C. for 72 h. After cooling to RT, the reaction was diluted with EtOAc (120 mL) and then washed with 0.5 M aq. NaOH (80 mL) followed by saturated aq. NaHCO₃ (80 mL) and brine (80 mL). The organic phase was dried over anhydrous MgSO₄ and concentrated under reduced pressure. The residue was purified by column chromatography (SiO₂, CH₂Cl₂) to provide 2-(5-methoxy-3,4-dihydronaphthalen-1-yl)acetonitrile (3.75 g, 41%) as a pale yellow crystalline solid. ¹H NMR (400 MHz, CDCl₃): δ 7.20 (ddt, J=8.5, 7.8, 0.7 Hz, 1H), 6.85 (dd, J=8.3, 1.0 Hz, 1H), 6.76 (d, J=7.8 Hz, 1H), 6.27 (tt, J=4.6, 1.6 Hz, 1H), 3.84 (s, 3H), 3.47 (q, J=1.8 Hz, 2H), 2.80 (t, J=8.3 Hz, 2H), 2.32 (tdt, J=8.2, 4.6, 1.9 Hz, 2H).

### Step 2: 2-(5-methoxynaphthalen-1-yl)acetonitrile (60)

To a solution of 2-(5-methoxy-3,4-dihydronaphthalen-1-yl)acetonitrile (2.05 g, 10.3 mmol) in DCE (60 mL) was added DDQ (3.04 g, 13.4 mmol) and the mixture was stirred at 100° C. under N₂ gas for 14 h. The reaction was then diluted with EtOAc (120 mL) and filtered through a pad of celite and the residue washed with EtOAc (2×25 mL). The combined filtrate was then washed with saturated aq. Na₂CO₃ (2×100 mL), dried over anhydrous MgSO₄ and concentrated under reduced pressure. The dark brown residue was purified by flash chromatography (SiO₂, 0-20% EtOAc in hexane) and the isolated material (1.30 g) was recrystalised from EtOAc/hexane to provide 2-(5-methoxynaphthalen-1-yl)acetonitrile (895 mg, 44%) as fine white needles. ¹H NMR (400 MHz, CDCl₃): δ 8.36-8.28 (m, 1H), 7.61 (dq, J=7.1, 1.0 Hz, 1H), 7.56-7.38 (m, 3H), 6.90 (dd, J=7.6, 0.9 Hz, 1H), 4.12 (br s, 2H), 4.02 (s, 3H); ¹³C NMR (400 MHz, CDCl₃): δ 156.3, 132.0, 127.4, 127.1, 126.1, 125.5, 124.9, 123.2, 117.9, 114.7, 104.6, 55.8, 22.2.

### Step 3: 2-(5-methoxynaphthalen-1-yl)ethan-1-amine (I-52)

To anhydrous THF (30 mL) at 0° C. was added LiAlH₄ (861 mg, 22.7 mmol) portionwise under an atmosphere of N₂. Maintaining 0° C., a solution of 2-(5-methoxynaphthalen-1-yl)acetonitrile (895 mg, 4.54 mmol) in anhydrous THF (10 mL) was added dropwise and the mixture was stirred at room temperature under N₂ atmosphere overnight. The reaction was cooled on an ice/water bath and quenched by slow addition of cold H₂O (1 mL) followed by 15% aq. NaOH (1 mL) and H₂O (3 mL). The mixture was filtered through a pad of celite which was subsequently washed through with THF. The filtrate was concentrated under reduced pressure and the residue purified by flash chromatography (SiO₂, 2-10% MeOH/NH₃ in CH₂Cl₂) to provide 2-(5-methoxynaphthalen-1-yl)ethan-1-amine (153 mg, 17%) as a white solid. ¹H NMR (400 MHz, DMSO-d₆): δ 8.04 (ddd, J=7.9, 1.9, 0.8 Hz, 1H), 7.66 (dt, J=8.6, 0.9 Hz, 1H), 7.45 (dd, J=8.6, 7.6 Hz, 1H), 7.42-7.35 (m, 2H), 6.97 (d, J=7.9 Hz, 1H), 3.96 (s, 3H), 3.10 (dd, J=8.5, 6.4 Hz, 2H), 2.85 (dd, J=8.3, 6.6 Hz, 2H).

### Step 4: 2-(5-methoxynaphthalen-1-yl)ethan-1-amine hydrochloride (I-52·HCl)

2-(5-methoxynaphthalen-1-yl)ethan-1-amine (59 mg, 0.29 mmol) was formulated as the hydrochloride salt according to general procedure A which was isolated as white crystals (43 mg, 62%). ¹H qNMR purity: 96.0% (ERETIC). ¹H NMR (400 MHz, DMSO-d₆): δ 8.23 (s, 3H), 8.14-8.08 (m, 1H), 7.72 (dt, J=8.6, 0.9 Hz, 1H), 7.50 (dd, J=8.6, 7.7 Hz, 1H), 7.47-7.40 (m, 2H), 7.01 (dd, J=7.8, 0.8 Hz, 1H), 3.97 (s, 3H), 3.41-3.32 (m, 2H), 3.11-3.02 (m, 2H). ¹³C NMR (101 MHz, DMSO-d₆): δ 155.4, 133.2, 132.3, 127.4, 126.6, 125.3, 124.9, 120.8, 115.6, 104.4, 55.7, 30.5.

### Example 50: 2-(5-methoxynaphthalen-1-yl)-N,N-dimethylethan-1-amine (I-50)

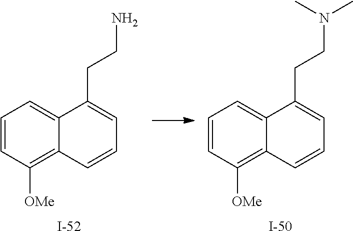

### Step 1: 2-(5-methoxynaphthalen-1-yl)-N,N-dimethylethan-1-amine (I-50)

To a solution of 2-(5-methoxynaphthalen-1-yl)ethan-1-amine (94 mg, 0.47 mmol) at 0° C. was added 40% w/w aq. formaldehyde (106 μL, 1.41 mmol) and NaCNBH₃ (88 mg, 1.41 mmol) and the mixture was stirred at RT for 3 h. The reaction was quenched by addition of 1 M aq. NaOH (2.0 mL) and then volatiles were removed under a stream of N₂ gas. The aqueous phase was extracted with Et₂O (3×10 mL), and the combined organics were washed with brine (15 mL), dried over anhydrous MgSO₄ and concentrated under reduced pressure. The crude material (92 mg) was purified by flash chromatography (SiO₂, 2-10% MeOH/NH₃ in CH₂Cl₂) to afford 2-(5-methoxynaphthalen-1-yl)-N,N-dimethylethan-1-amine (37 mg, 35%) as a yellow oil.

### Step 2: 2-(5-methoxynaphthalen-1-yl)-N,N-dimethylethan-1-amine fumarate (I-50·Fum)

2-(5-methoxynaphthalen-1-yl)-N,N-dimethylethan-1-amine (53 mg, 0.45 mmol) was formulated as the fumarate salt according to general procedure B which was isolated as white crystals (36 mg, 69%). ¹H qNMR purity: 98.6% (ERETIC); ¹H NMR (400 MHz, DMSO-d₆): δ 8.12-8.03 (m, 1H), 7.66 (dt, J=8.6, 0.8 Hz, 1H), 7.47 (dd, J=8.6, 7.7 Hz, 1H), 7.45-7.38 (m, 2H), 6.99 (dd, J=7.8, 0.8 Hz, 1H), 6.57 (s, 2H), 3.96 (s, 3H), 3.33-3.24 (m, 2H), 2.93-2.85 (m, 2H), 2.53 (s, 6H); ¹³C NMR (101 MHz, DMSO-d₆): δ 167.2, 155.3, 134.7, 134.7, 132.4, 127.2, 126.4, 125.2, 124.9, 120.4, 115.7, 104.2, 58.4, 55.6, 43.4, 29.1.

### Example 51: Functional Assays 5-HT_{2A}, 5-HT_{2B} and 5-HT_{2C} Receptors

Activity at 5-HT_{2A}, 5-HT_{2B} and 5-HT_{2C} receptors was determined using a FLIPR Ca²⁺ flux assay at WuXi AppTec Co. Ltd. (Hong Kong) Discovery Biology Unit according to their standard protocols. Briefly, stably transfected cells expressing the receptor of interest (HEK293 for 5-HT_{2A} and 5-HT_{2C}; CHO-K1 for 5-HT_{2B}) were grown and plated in a 384 well plate and incubated at 37° C. and 5% CO₂ overnight. A 250 mM stock solution of probenecid in FLIPR calcium assay buffer (10 mL) was freshly prepared and combined with a fluorescent dye (Fluo-4 Direct) to give a final assay concentration of 2.5 mM. Reference compounds were 4-fold serially diluted and the screening compounds were 3-fold serially diluted in 100% DMSO for 10 points using Agilent Bravo, and 750 nL was added to a 384 well compound plate using Echo along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read. Results are provided in Table 1 below.

TABLE 1
Agonist activity of exemplified compounds at selected serotonin
(5-HT) receptors in Ca²⁺ flux functional assays.

| | 5-HT_{2A} | | 5-HT_{2B} | | 5-HT_{2C} | |
|---|---|---|---|---|---|---|
| Example | EC₅₀ nM | Emax (%) | EC₅₀ nM | Emax (%) | EC₅₀ nM | Emax (%) |
| I-1 | 96 | 79 | >10000 | −0.99 | 33 | 82 |
| I-2 | 360 | 70 | >10000 | 12 | 733 | 88 |
| I-3 | 234 | 77 | 225 | 72 | 944 | 97 |
| I-4 | 446 | 111 | 1196 | 123 | 1260 | 99 |
| I-5 | 86 | 63 | >10000 | 17 | 43 | 94 |
| I-6 | 125 | 81 | >10000 | 24 | 79 | 95 |
| I-7 | 164 | 84 | >10000 | 36 | 603 | 56 |
| I-15 | 133 | 70 | | | 292 | 65 |
| I-17 | 92 | 84 | | | 18 | 97 |
| I-13 | 138 | 49 | >10000 | 5 | 582 | 58 |
| I-21 | 53 | 73 | | | 12 | 90 |
| I-22 | 457 | 85 | >10000 | 34 | 157 | 95 |
| I-23 | 165 | 93 | 103 | 89 | 291 | 88 |
| I-24 | 1467 | 98 | 4704 | 116 | 2563 | 90 |
| I-25 | 158 | 67 | >10000 | 24 | 201 | 84 |
| I-26 | 41 | 68 | | | 15 | 86 |
| I-27 | 213 | 86 | >10000 | 2 | 211 | 75 |
| I-28 | 191 | 94 | >10000 | 46 | 766 | 76 |
| I-29 | 36 | 79 | | | 54 | 84 |
| I-30 | 496 | 48 | | | 409 | 60 |
| I-31 | 60 | 64 | | | 123 | 57 |
| I-32 | 240 | 63 | | | 61 | 87 |
| I-33 | 204 | 58 | | | 115 | 67 |
| I-53 | 34 | 83 | | | 1 | 97 |
| I-38 | 245 | 66 | | | 46 | 83 |
| I-39 | 168 | 58 | | | 33 | 93 |
| I-40 | 378 | 68 | | | 120 | 84 |
| I-41 | 1025 | 33 | | | 150 | 80 |
| I-50 | | 28% @ | | | | |
| | | 10 uM | | | | |
| I-52 | 295 | 79 | | | 289 | 91 |
| I-8 | NA | 24% @ | >10000 | 8 | 314 | 87 |
| | | 10 uM | | | | |
| I-9 | 1842 | 39 | >10000 | 1 | 2386 | 65 |

### Example 49: In Vivo Pharmacokinetics Experiments

The study was conducted using established procedures in accordance with the Australian Code of Practice for the Care and Use of Animals for Scientific Purposes, and the study protocols were reviewed and approved by the Monash Institute of Pharmaceutical Sciences Animal Ethics Committee.

The systemic exposure of selected examples was studied in non-fasted male C57BL/6 mice weighing between 22.3-26.2 g. Mice had access to food and water ad libitum throughout the pre- and post-dose sampling period.

On the day of dosing, the formulation of each compound was prepared by dissolving solid compound in phosphate buffer saline (50 mM) using vortexing, creating colourless solutions (pH 6.1-6.3) for each compound.

Compounds were dosed to mice by IP injection (10 mL/kg dose volume via a 27G needle; n=9 mice per compound) and blood samples were collected at 5 and 30 min; 1, 2 and 4 h post-dose (n=3 mice per time point for each compound). A maximum of three blood samples were obtained from each mouse, with plasma samples being taken via submandibular bleed (approximately 120 μL). Once collected, blood samples were centrifuged immediately, supernatant plasma was removed, and stored at −80° C. until analysis by LCMS. In addition, at the 5 and 30 min and 4 h post-dose time points, the whole brain was rapidly removed from the carcass soon after the blood collection. The whole brains were blotted to remove excess blood, placed into pre-weighed polypropylene vials, and weighed. The brains were snap frozen in dry ice and subsequently stored frozen (−80° C.) until analysis.

### Bioanalytical Method Summary:

Concentrations of test compound in plasma and tissue samples were determined using an LCMS/MS method validated for linearity, accuracy, precision, matrix factor and recovery (Table 2).

Test compound standard solutions were diluted from a concentrated stock solution (1 mg/mL in DMSO) using 50% acetonitrile in water (v/v) and a calibration curve was prepared in a matched matrix to the test samples.

Plasma: The plasma calibration curve was prepared by spiking aliquots of blank mouse plasma (25 μL) with test compound standard solutions (5 μL) and internal standard solution (5 μL of diazepam, 5 μg/mL in 50% acetonitrile in water). Test plasma samples (25 μL) were thawed, mixed, and then spiked with internal standard solution (5 μL). Plasma protein precipitation was performed by addition of acetonitrile (3-fold volume ratio) and thorough vortex mixing. Samples were centrifuged (RCF=9391×g) for 3 minutes and the supernatant (90 μL) was collected for analysis.

Tissue: Pre-weighed tissue samples (brain) were homogenised using a glass rod in buffer containing an EDTA/potassium fluoride solution (0.1 M/4 mg/mL) as a stabilisation cocktail to minimise the potential for ex vivo degradation (3 mL cocktail/g tissue). The tissue homogenate was briefly centrifuged (RCF=79×g) for 10 seconds to separate the foam layer before transferring an aliquot of the tissue homogenate (200 μL) to a fresh Eppendorf tube for sample extraction. Calibration standards were prepared by spiking blank brain homogenate (200 μL) with the solution standards (10 μL) and the internal standard (10 μL). Study samples were similarly prepared, except that acetonitrile (10 μL) was added instead of solution standards to maintain the same volume. Protein precipitation was carried out by the addition of a 3-fold volume of acetonitrile, followed by vortex mixing and centrifugation (RCF=9391×g) for 3 min to recover the supernatant for analysis.

Replicate analysis: Triplicate analytical replicate (ARs) samples were prepared similarly to the standards for each sample type at three concentrations (50, 500 and 2,000 ng/mL) and repeat injections of these ARs were included throughout the analytical run to assess assay performance. The extraction of the test compound from the standards and ARs were conducted as described above.

All test samples were quantified within the calibration range of the assay and the assay performance for ARs were deemed acceptable. The stability of each test compound was confirmed in homogenate during the period of sample processing (15 min).

TABLE 2
Summary of bioanalytical method for I-7 & I-15

| | |
|---|---|
| Instrument | Waters Xevo TQS Micro coupled to a Waters Acquity UPLC |
| Detection | Positive electrospray ionisation multiple-reaction monitoring mode |
| Column | Kinetex 2.6 u PFP 100A column (50 × 2.1 mm, 2.6 μm) |
| LC Conditions | Gradient cycle time: 2 min; Injection vol: 0.3 μL; |
| | Flow rate: 0.8 mL/min |
| Mobile | (A) 0.005M ammonium formate in water; (B) 0.05% ammonium |
| Phase | formate in methanol |
| Sample | Plasma: Protein precipitation using acetonitrile (1:5 volume ratio) |
| Preparation | Tissue: Protein precipitation using acetonitrile (1:3 volume ratio) |

| | t_{R}* | Transition | Cone | CID^{#} |
|---|---|---|---|---|
| Analyte | (min) | (m/z) | Voltage (V) | (V) |
| I-7 | 1.32 | 258.09 > 85.95 | 20 | 20 |
| I-15 | 1.23 | 256.07 > 184.98 | 20 | 20 |
| Diazepam (IS) | 0.93 | 285.15 > 193.10 | 40 | 25 |

The highest abundance product ion with minimum interference with the matrix were selected for quantification. Data acquisition was performed using MassLynx software (V4.2).

IS: Internal standard|*Retention time|#Collision-Induced Dissociation

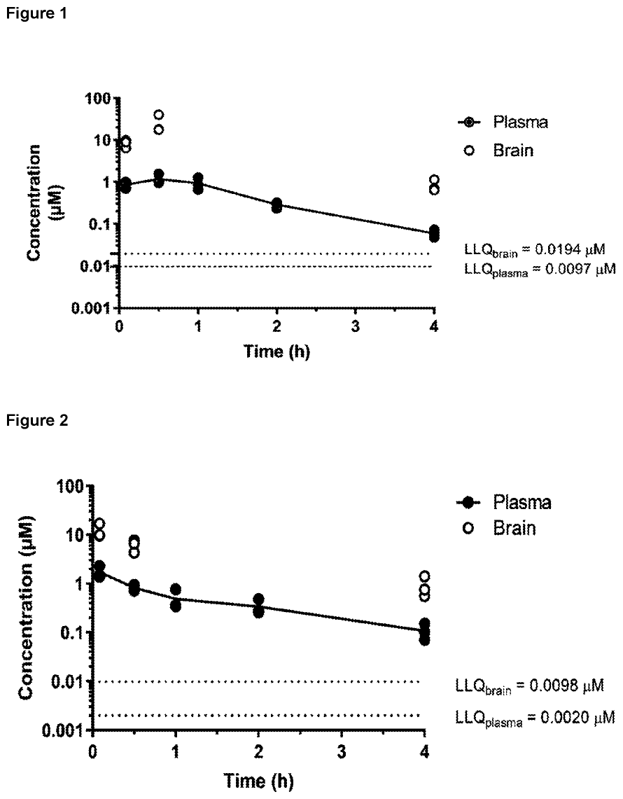

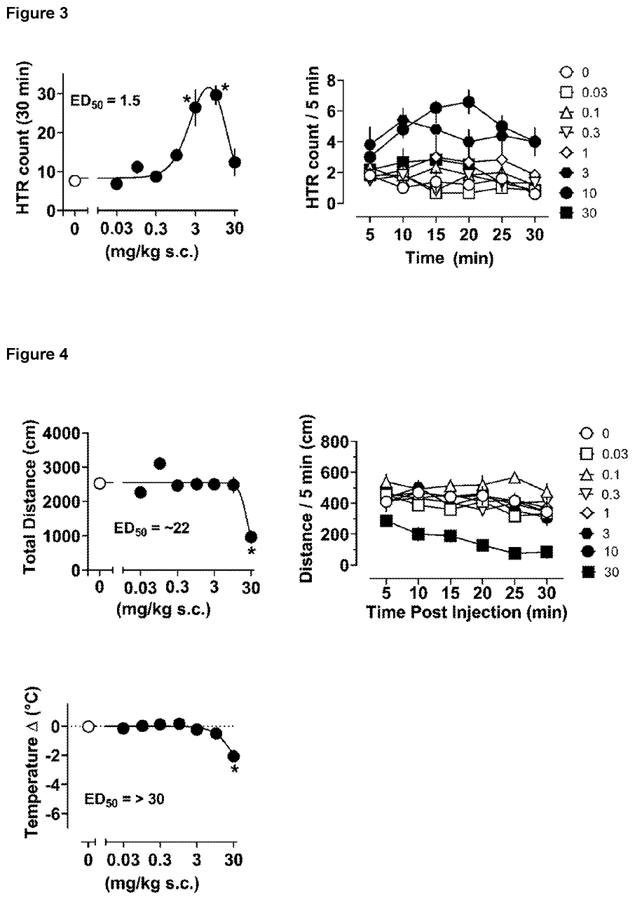

Maximal plasma concentration of 1-7 following IP administration at 10 mg/kg are shown in Table 3. Comprehensive pharmacokinetic data including brain penetration information is displayed in FIG. 1 and/or Table 4.

TABLE 3
Exposure parameters for I-7 & I-15 in male C57BL/6
mice following IP administration at 10 mg/kg.

| Parameter | I-7 | I-15 |
|---|---|---|
| Apparent t_{1/2} (h) | 0.77 | 1.2^{c} |
| Plasma Cₘₐₓ (μM) | 1.18 | 1.72 |
| Tₘₐₓ | 0.50 h | 5 min |
| Plasma AUC_{0-4 h} (h*μM) | 1.82 | 1.71 |
| Plasma AUC_{0-inf} (h*μM) | 1.89 | 1.90^{c} |
| Brain AUC_{0-4 h} (h*μM) | 32.3 | 13.8 |
| (Total) time-averaged B:P^{a} | 18 | 8.1 |
| (Unbound) time-averaged B:P^{b} | 3.2 | 2.0 |

^{a}Calculated as AUC_{brain, 0-4 h}/AUC_{plasma, 0-4 h}
^{b}Calculated as (fu, brain * AUCbrain, 0-4 h)/(fu, plasma * AUCplasma, 0-4 h)
^{c}The terminal elimination phase of the profile has been estimated on the basis of the last two timepoints therefore values based on extrapolation to infinity are approximations only

TABLE 4
Individual and mean ± SD (n = 3) plasma and brain concentrations, and brain-to-
plasma (B:P) ratios, of I-7 & I-15 in male C57BL/6 mice following IP administration at 10 mg/kg.

| | | Plasma Concentration | | Brain Parenchyma | | | |
|---|---|---|---|---|---|---|---|
| | | (μM) | | Concentration (μM) | | B:P Ratio | |
| Time | Mouse | | Mean ± | | Mean ± | | Mean ± |
| (h) | ID | Individual | SD | Individual | SD | Individual | SD |

I-7

| | | | | | | | |
|---|---|---|---|---|---|---|---|
| 0.083 | 1 | 0.695 | 0.864 ± 0.155 | 6.41 | 8.4 ± 1.79 | 9.2 | 9.7 ± 1.2 |
| | 2 | 0.896 | | 9.90 | | 11 | |
| | 3 | 1.00 | | 8.88 | | 8.9 | |
| 0.5 | 4 | 1.03 | 1.18 ± 0.329 | 17.9 | 25.2 ± 13.0 | 17 | 21 ± 4.6 |
| | 5 | 0.947 | | 17.5 | | 18 | |
| | 6 | 1.55 | | 40.1 | | 26 | |
| 4 | 7 | 0.0723 | 0.0592 ± 0.0121 | 1.14 | 0.0825 ± 0.275 | 16 | 14 ± 2.4 |
| | 8 | 0.0483 | | 0.696 | | 14 | |
| | 9 | 0.0571 | | 0.639 | | 11 | |

I-15

| | | | | | | | |
|---|---|---|---|---|---|---|---|
| 0.083 | 1 | 2.30 | 1.72 ± 0.506 | 16.9 | 12.1 ± 4.14 | 7.3 | 7.0 ± 0.33 |
| | 2 | 1.38 | | 9.59 | | 6.9 | |
| | 3 | 1.48 | | 9.89 | | 6.7 | |
| 0.5 | 4 | 0.718 | 0.823 ± 0.107 | 4.3 | 6.18 ± 1.70 | 6.0 | 7.4 ± 1.2 |
| | 5 | 0.932 | | 7.6 | | 8.2 | |
| | 6 | 0.820 | | 6.64 | | 8.1 | |
| 4 | 7 | 0.0701 | 0.107 ± 0.0406 | 0.553 | 0.905 ± 0.446 | 7.9 | 8.2 ± 1.0 |
| | 8 | 0.151 | | 1.41 | | 9.3 | |
| | 9 | 0.101 | | 0.755 | | 7.5 | |

### Example 54: Biotelemetry and Head-Twitch Response (HTR) Experiments

Mice (C57BL/6J males) were purchased from the Jackson Laboratory (Bar Harbor, ME, USA) at 5-6 weeks of age and allowed at least 1-2 weeks to acclimate to the NIDA, Intramural Research Program (IRP), animal research facility in Baltimore, MD, USA. The animal facility is fully accredited by the Association for the Assessment and Accreditation of Laboratory Animal Care, and all procedures were approved by the NIDA IRP Animal Care and Use Committee. Mice were initially group housed 3-5 per cage during acclimation and housed in a 12 h light-dark cycle throughout the study, with lights on at 0700 h. Food and water were available ad libitum except during testing. Cohorts of 20-24 mice were used for each test drug. The mice were subjected to experimental testing once every 1-2 weeks for 2-3 months to complete dose-effect curves and antagonist experiments. A minimum of 7 days between treatments was utilized to avoid any tolerance to effects of repeated drug administration. All drug doses represent the weight of the salt dissolved in 0.9% saline vehicle. Mice were tested first in dose-response studies to assess the effects of each compound at doses from 0.03 to 30 mg/kg s.c. and were subsequently tested in antagonist reversal studies utilizing pretreatment with M100907 and WAY100635. All experiments were conducted from 0900 to 1700 local time during the light phase, as sensitivity of rodents to other tryptamine psychedelics is diurnal, with maximal HTR observed in the middle of the light phase. Experiments were run during the light phase also to avoid any potential influence of melatonin receptor activity on HTR as melatonin and related agonists are known to reduce HTR induced by DOI in rats. For each experiment, mice were acclimated to the testing room in their home cage for at least 1 h prior to experimental sessions. Behavioral test sessions were carried out in Tru Scan mouse locomotor arenas equipped with photobeam arrays (Coulbourn Instruments, Holliston, MA, USA), which were modified with cylindrical inserts and transparent floors useful in detecting mouse HTR.

Subcutaneous Temperature Transponder Implants. At least 1 week prior to the start of the experiments, mice received s.c. implanted temperature transponders (14×2 mm, model IPTT-300, Bio Medic Data Systems, Inc., Seaford, DE, USA) under brief isoflurane anesthesia. Mice were single housed post implant for the remainder of the study to protect the transponder from removal by cage mates. Temperature was determined noninvasively using a handheld receiver that is sensitive to signals emitted from the implanted transponders.

Prior to each experiment, mouse body weight and temperature were recorded. Mice were then placed into testing chambers for acclimation. In dose-response studies, after a brief 5 min acclimation, mouse body temperature was recorded for baseline measurement, mice received s.c. injection of test substance or vehicle, and animals were returned to the testing arena for 30 min. During the session, locomotor activity was monitored via photobeam tracking of movements in the horizontal plane to yield distance traveled in centimeter. HTR was monitored by the analysis of GoPro Hero Black 7 video recordings (120 frames per sec and 960p resolution) using a commercially available software package from Clever Sys Inc. (Reston, VA, USA).82 Post-treatment body temperature values were also recorded, and temperature data are represented as change from pretreatment baseline.

In antagonist reversal experiments, mice received a s.c. injection of either receptor antagonists or vehicle and were returned to the testing chamber for 30 min. During this period, locomotor activity was monitored to examine the potential effects of antagonist treatment on general behavior or movement. At 30 min after antagonist administration, mice were given test drug or vehicle and returned to the chambers for an additional 30 min of video recording used for analyses.

All statistical analyses were conducted using GraphPad Prism 9 (La Jolla, CA, USA). Dose-response data from mouse experiments were analyzed using nonlinear regression, and potency values were determined from the rising phase of the curves for HTR measures. For mouse studies, one-way ANOVA with Dunnett's post hoc test was used to compare all conditions to vehicle controls (0 or 0,0) in dose-response and antagonist experiments. Time-course drug effects for all parameters in mouse studies are shown for reference. Mean HTR count, distance travelled, and temperature change for each condition were used for statistical comparisons. Alpha was set at 0.05 for all analyses.

1. A compound of formula (I): 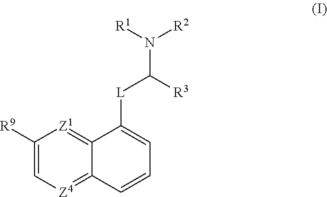
or a pharmaceutically acceptable salt, solvate, tautomer, N-oxide, stereoisomer, metabolite, polymorph and/or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ alkoxy, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² together with the nitrogen atom to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² together with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
Z¹ is CR⁸ or N;
Z⁴ is CR¹¹ or N;
R⁸, R⁹ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

2. The compound of claim 1, wherein R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₇₋₁₈ alkylenearyl and C₄₋₁₄ alkylenecycloalkyl.

3. The compound of claim 2, wherein R¹ and R² are each independently selected from hydrogen, C₁₋₄ alkyl, C₃₋₄ cycloalkyl and C₇₋₈ alkylenearyl.

4. The compound of claim 3, wherein R¹ and R², together with the nitrogen to which they are attached, form any one of the following:

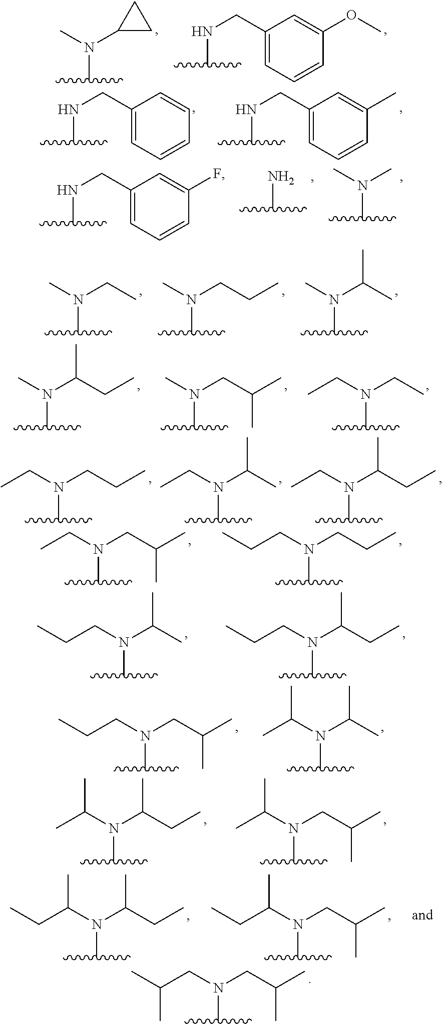

5. The compound of claim 1, wherein R¹ and R² together with the nitrogen atom to which they are attached to form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in claim 1.

6. The compound of any one of claims 1 to 5, wherein R³ is hydrogen.

7. The compound of claim 1, wherein R³ and one of R¹ and R² together with the atoms to which they are attached to form a C₃₋₈ heterocycloalkyl, said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in claim 1.

8. The compound of any one of claims 1 to 7, wherein R⁸, R⁹ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O)R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in claim 1.

9. The compound of claim 8, wherein one of R⁸, R⁹ and R¹¹ when present are each independently selected from halogen, CN, C₁₋₆ alkyl, C₁₋₆ haloalkyl and OR¹³ wherein R¹³ is selected from hydrogen, C₁₋₆ alkyl and C₁₋₆ haloalkyl, and the other of R⁸, R⁹ and R¹¹ are each hydrogen.

10. The compound of claim 9, wherein one of R⁸, R⁹ and R¹¹ when present is fluoro, chloro, hydroxy or OCH₃, and the other of R⁸, R⁹ and R¹¹ are each hydrogen.

11. The compound of any one of claims 1 to 10, wherein L is C₁₋₄ alkylene.

12. The compound of claim 11, wherein L is methylene.

13. The compound of any one of claims 1 to 12 having the formula (Ia): 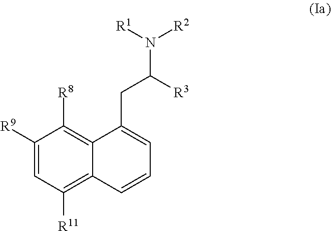
wherein R¹, R², R³, R⁸, R⁹ and R¹¹ are as defined in any one of claims 1 to 10.

14. The compound of any one of claims 1 to 12, wherein Z¹ is N.

15. The compound of claim 14 having the formula (Ib): 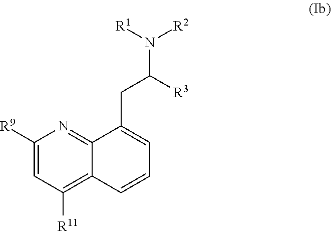
wherein R¹, R², R³, R⁹ and R¹¹ are as defined in any one of claims 1 to 10.

16. The compound of any one of claims 1 to 12, wherein Z⁴ is N.

17. The compound of claim 16 having the formula (Ic): 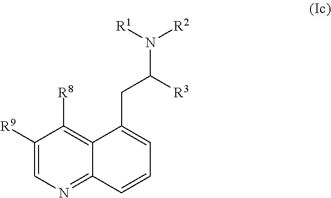
wherein R¹, R², R³, R⁸ and R⁹ are as defined in any one of claims 1 to 10.

18. The compound of claim 1 selected from any one of the following: 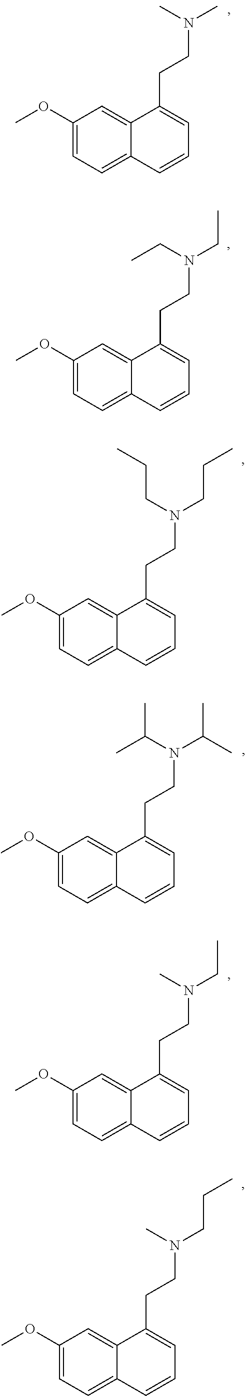 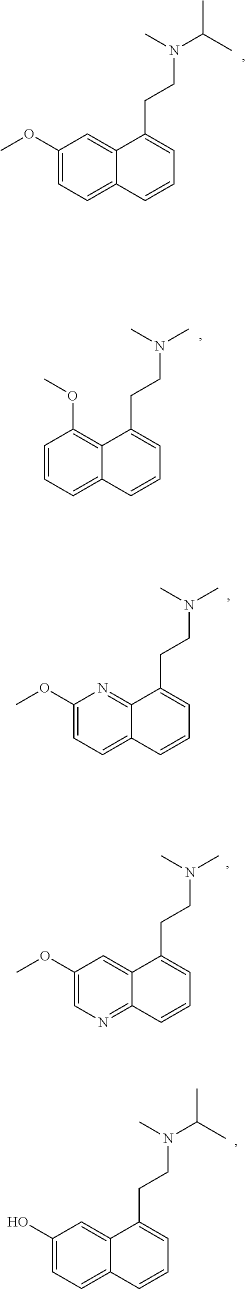 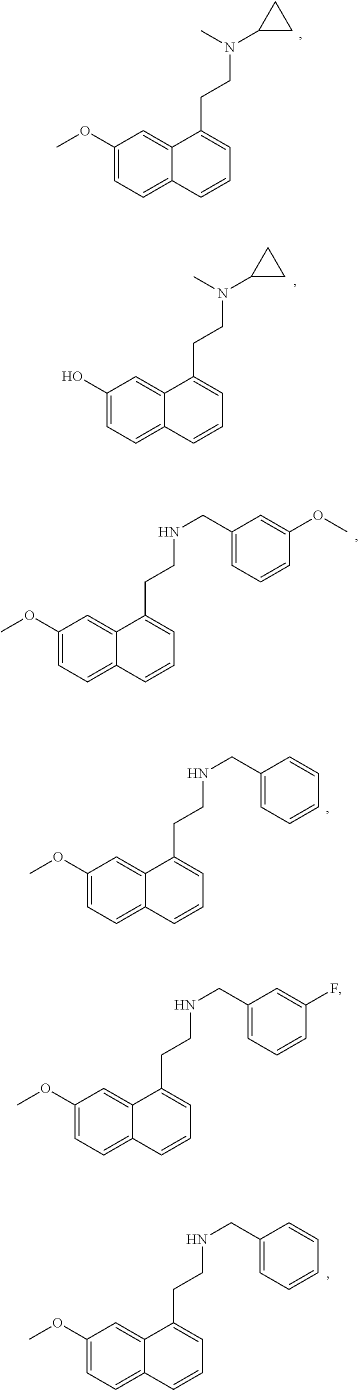 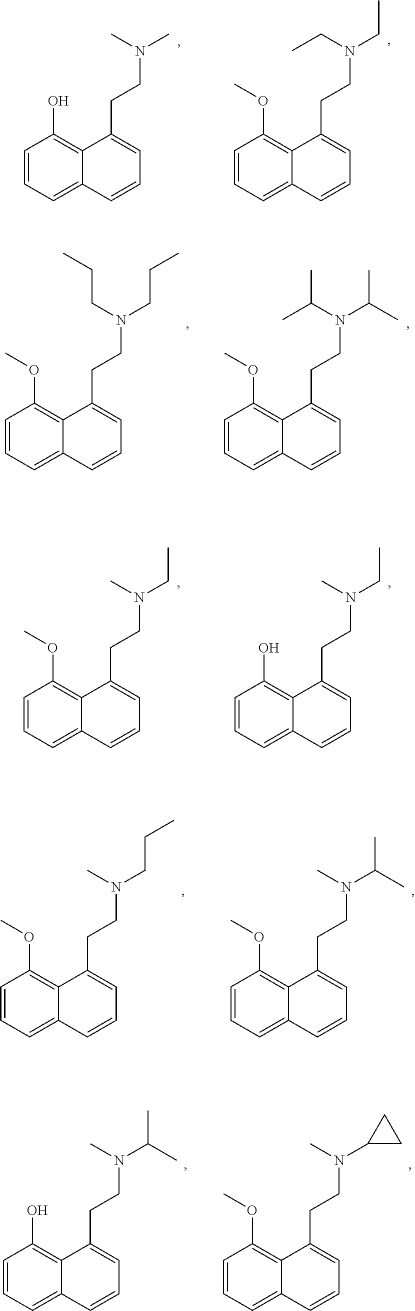 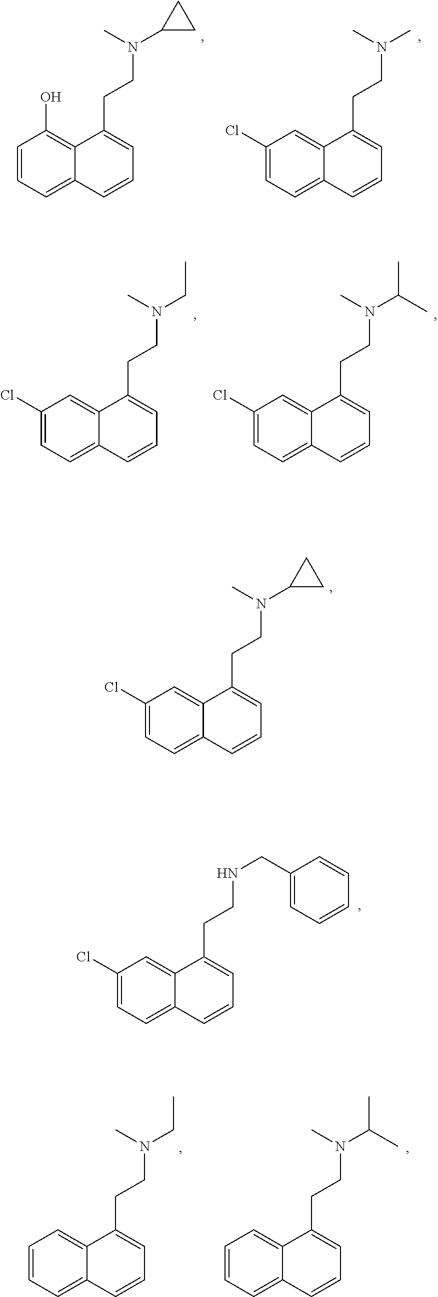 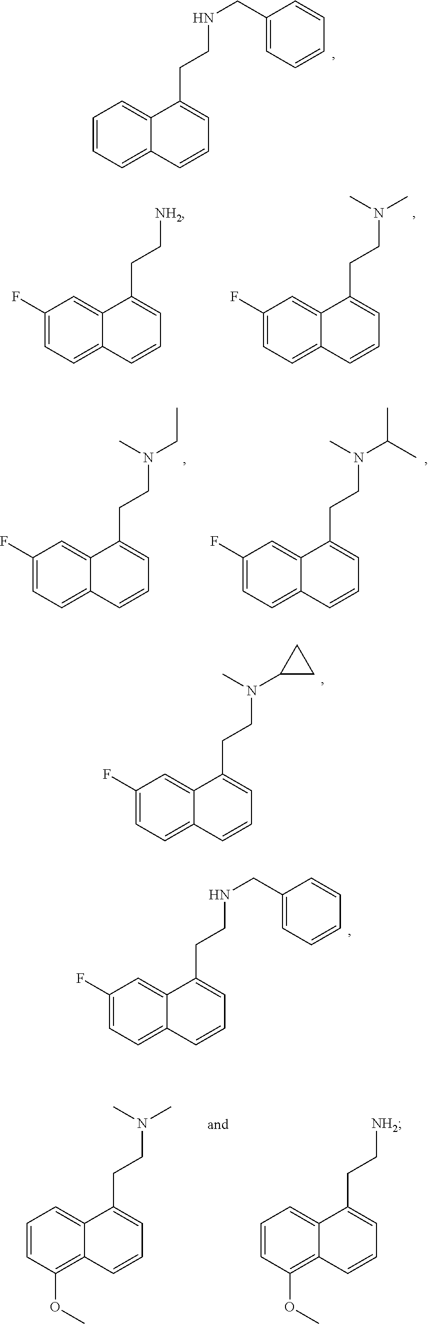
or a pharmaceutically acceptable salt, solvate, tautomer, N-oxide, stereoisomer, metabolite, polymorph and/or prodrug thereof.

19. A medicament comprising a compound of any one of claims 1 to 18, or a pharmaceutically acceptable salt, solvate, tautomer, N-oxide, stereoisomer, metabolite, polymorph and/or prodrug thereof.

20. A pharmaceutical composition comprising a compound of any one of claims 1 to 18, or a pharmaceutically acceptable salt, solvate, tautomer, N-oxide, stereoisomer, metabolite, polymorph and/or prodrug thereof, and a pharmaceutically acceptable excipient.

21. A method of treating a disease, disorder or condition associated with activity of a serotonin receptor, the method comprising administering to a subject in need thereof a compound of any one of claims 1 to 18, or a pharmaceutically acceptable salt, solvate, tautomer, N-oxide, stereoisomer, metabolite, polymorph and/or prodrug thereof.

22. A method of treating a disease, disorder or condition associated with activity of a serotonin receptor, the method comprising administering to a subject in need thereof a compound of any one of claims 1 to 18, or a pharmaceutically acceptable salt, solvate, tautomer, N-oxide, stereoisomer, metabolite, polymorph and/or prodrug thereof, in combination with another agent useful for treatment of said disease, disorder or condition.

23. A method of treating a mental illness, the method comprising administering to a subject in need thereof a compound of any one of claims 1 to 18, or a pharmaceutically acceptable salt, solvate, tautomer, N-oxide, stereoisomer, metabolite, polymorph and/or prodrug thereof.

24. The method of claim 23, wherein the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

25. A method for treating a central nervous system (CNS) disease, disorder or condition and/or a neurological disease, disorder or condition, the method comprising administering to a subject in need thereof a compound of any one of claims 1 to 18, or a pharmaceutically acceptable salt, solvate, tautomer, N-oxide, stereoisomer, metabolite, polymorph and/or prodrug thereof.

26. The method of claim 25, wherein the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

27. A method for increasing neuronal plasticity and/or increasing dendritic spine density, the method comprising contacting a neuronal cell with a compound of any one of claims 1 to 18, or a pharmaceutically acceptable salt, solvate, tautomer, N-oxide, stereoisomer, metabolite, polymorph and/or prodrug thereof, in an amount sufficient to increase neuronal plasticity and/or increase dendritic spine density of the neuronal cell.
